SEQUENCE LISTING (302 sequences)

SEQ ID NO: 1 - DNA, 18 bp, synthetic construct
  misc_feature 1..18: note = MOX F'
  source 1..18: mol_type = other DNA
agaccctgttcgagatag

SEQ ID NO: 2 - DNA, 18 bp, synthetic construct
  misc_feature 1..18: note = MOX R'
  source 1..18: mol_type = other DNA
atggtgatgctgtcaaag

SEQ ID NO: 3 - DNA, 20 bp, synthetic construct
  misc_feature 1..20: note = MOX-FAM
  source 1..20: mol_type = other DNA
cgtgagcaagaccctgactg

SEQ ID NO: 4 - DNA, 20 bp, synthetic construct
  misc_feature 1..20: note = FOX F'
  source 1..20: mol_type = other DNA
actatttcaactatggggtt

SEQ ID NO: 5 - DNA, 18 bp, synthetic construct
  misc_feature 1..18: note = TTG TCA TCC AGC TCA AAG
  source 1..18: mol_type = other DNA
ttgtcatccagctcaaag

SEQ ID NO: 6 - DNA, 18 bp, synthetic construct
  misc_feature 1..18: note = FOX-TEX
  source 1..18: mol_type = other DNA
tgaccgcagcataggcac

SEQ ID NO: 7 - DNA, 18 bp, synthetic construct
  misc_feature 1..18: note = EBC F'
  source 1..18: mol_type = other DNA
gtggcggtgatttatgag

SEQ ID NO: 8 - DNA, 23 bp, synthetic construct
  misc_feature 1..23: note = EBC R'
  source 1..23: mol_type = other DNA
cggtgaaggttttacttatagaa

SEQ ID NO: 9 - DNA, 19 bp, synthetic construct
  misc_feature 1..19: note = EBC-HEX
  source 1..19: mol_type = other DNA
cagccgcactacttcacct

SEQ ID NO: 10 - DNA, 21 bp, synthetic construct
  misc_feature 1..21: note = DHA F'
  source 1..21: mol_type = other DNA
tgcgtacggttatgagaacaa

SEQ ID NO: 11 - DNA, 18 bp, synthetic construct
  misc_feature 1..18: note = DHA R'
  source 1..18: mol_type = other DNA
cccagcgcagcatatctt

SEQ ID NO: 12 - DNA, 24 bp, synthetic construct
  misc_feature 1..24: note = DHA-FAM
  source 1..24: mol_type = other DNA
atgcggaatcttacggcgtggaat

SEQ ID NO: 13 - DNA, 19 bp, synthetic construct
  misc_feature 1..19: note = CMY F'
  source 1..19: mol_type = other DNA
tccagcgttattgatatgg

SEQ ID NO: 14 - DNA, 18 bp, synthetic construct
  misc_feature 1..18: note = CMY R'
  source 1..18: mol_type = other DNA
catctcccagcctaatcc

SEQ ID NO: 15 - DNA, 21 bp, synthetic construct
  misc_feature 1..21: note = CMY-TEX
  source 1..21: mol_type = other DNA
acatatcgccaatacgccagt

SEQ ID NO: 16 - DNA, 20 bp, synthetic construct
  misc_feature 1..20: note = ACC F'
  source 1..20: mol_type = other DNA
gccgctgatgcagaagaata

SEQ ID NO: 17 - DNA, 20 bp, synthetic construct
  misc_feature 1..20: note = ACC R'
  source 1..20: mol_type = other DNA
tttgccgctaacccatagtt

SEQ ID NO: 18 - DNA, 11 bp, synthetic construct
  misc_feature 1..11: note = ACC-HEX
  source 1..11: mol_type = other DNA
ccgacataccg

SEQ ID NO: 19 - DNA, 20 bp, synthetic construct
  misc_feature 1..20: note = IC F'
  source 1..20: mol_type = other DNA
gagaggatgaccagccacac

SEQ ID NO: 20 - DNA, 21 bp, synthetic construct
  misc_feature 1..21: note = IC R'
  source 1..21: mol_type = other DNA
agtactttacaacccgaaggc

SEQ ID NO: 21 - DNA, 25 bp, synthetic construct
  misc_feature 1..25: note = IC-TYE
  source 1..25: mol_type = other DNA
tgagacacggtccagactcctacgg

SEQ ID NO: 22 - DNA, 18 bp, synthetic construct
  misc_feature 1..18: note = CTX-M-14 F'
  source 1..18: mol_type = other DNA
ttggtgacgtggctcaaa

SEQ ID NO: 23 - DNA, 21 bp, synthetic construct
  misc_feature 1..21: note = CTX-M-14 R'
  source 1..21: mol_type = other DNA
atatcattggtggtgccgtag

SEQ ID NO: 24 - DNA, 24 bp, synthetic construct
  misc_feature 1..24: note = CTX-M-14-FAM
  source 1..24: mol_type = other DNA
cgtggactgtgggtgataagaccg

SEQ ID NO: 25 - DNA, 21 bp, synthetic construct
  misc_feature 1..21: note = CTX-M-15 F'
  source 1..21: mol_type = other DNA
gtcacgctgttgttaggaagt

SEQ ID NO: 26 - DNA, 21 bp, synthetic construct
  misc_feature 1..21: note = CTX-M-15 R'
  source 1..21: mol_type = other DNA
taatcaatgccacacccagtc

SEQ ID NO: 27 - DNA, 24 bp, synthetic construct
  misc_feature 1..24: note = CTX-M-15-TEX615
  source 1..24: mol_type = other DNA
aacttgccgaattagagcggcagt

SEQ ID NO: 28 - DNA, 24 bp, synthetic construct
  misc_feature 1..24: note = OXA48-F'
  source 1..24: mol_type = other DNA
agcagcaaggatttaccaataatc

SEQ ID NO: 29 - DNA, 21 bp, synthetic construct
  misc_feature 1..21: note = OXA48-R'
  source 1..21: mol_type = other DNA
cgtctgtccatcccacttaaa

SEQ ID NO: 30 - DNA, 24 bp, synthetic construct
  misc_feature 1..24: note = OXA48-HEX
  source 1..24: mol_type = other DNA
tagcttgatcgccctcgatttggg

SEQ ID NO: 31 - DNA, 19 bp, synthetic construct
  misc_feature 1..19: note = CMY F'
  source 1..19: mol_type = other DNA
tccagcgttattgatatgg

SEQ ID NO: 32 - DNA, 18 bp, synthetic construct
  misc_feature 1..18: note = CMY R'
  source 1..18: mol_type = other DNA
catctcccagcctaatcc

SEQ ID NO: 33 - DNA, 21 bp, synthetic construct
  misc_feature 1..21: note = CMY-TxR
  source 1..21: mol_type = other DNA
acatatcgccaatacgccagt

SEQ ID NO: 34 - DNA, 19 bp, synthetic construct
  misc_feature 1..19: note = NDM F'
  source 1..19: mol_type = other DNA
tttgatcgtcagggatggc

SEQ ID NO: 35 - DNA, 21 bp, synthetic construct
  misc_feature 1..21: note = NDM R'
  source 1..21: mol_type = other DNA
caggttgatctcctgcttgat

SEQ ID NO: 36 - DNA, 22 bp, synthetic construct
  misc_feature 1..22: note = NDM-HEX
  source 1..22: mol_type = other DNA
agaccgcccagatcctcaactg

SEQ ID NO: 37 - DNA, 21 bp, synthetic construct
  misc_feature 1..21: note = KPC F'
  source 1..21: mol_type = other DNA
cgctaaactcgaacaggactt

SEQ ID NO: 38 - DNA, 21 bp, synthetic construct
  misc_feature 1..21: note = KPC R'
  source 1..21: mol_type = other DNA
taacttacagttgcgcctgag

SEQ ID NO: 39 - DNA, 24 bp, synthetic construct
  misc_feature 1..24: note = KPC-FAM
  source 1..24: mol_type = other DNA
atcggtgtgtacgcgatggatacc

SEQ ID NO: 40 - DNA, 19 bp, synthetic construct
  misc_feature 1..19: note = VIM F'
  source 1..19: mol_type = other DNA
cattcgaccgacaacttag

SEQ ID NO: 41 - DNA, 17 bp, synthetic construct
  misc_feature 1..17: note = VIM R'
  source 1..17: mol_type = other DNA
cgtgcgtgacaactcat

SEQ ID NO: 42 - DNA, 24 bp, synthetic construct
  misc_feature 1..24: note = VIM-TEX
  source 1..24: mol_type = other DNA
tgtgctctatggtggttgtgcgat

SEQ ID NO: 43 - DNA, 21 bp, synthetic construct
  misc_feature 1..21: note = DHA F'
  source 1..21: mol_type = other DNA
tgcgtacggttatgagaacaa

SEQ ID NO: 44 - DNA, 18 bp, synthetic construct
  misc_feature 1..18: note = DHA R'
  source 1..18: mol_type = other DNA
cccagcgcagcatatctt

SEQ ID NO: 45 - DNA, 24 bp, synthetic construct
  misc_feature 1..24: note = DHA-FAM
  source 1..24: mol_type = other DNA
atgcggaatcttacggcgtgaaat

SEQ ID NO: 46 - DNA, 21 bp, synthetic construct
  misc_feature 1..21: note = IMP F'
  source 1..21: mol_type = other DNA
acgtagtggtttggttacctg

SEQ ID NO: 47 - DNA, 22 bp, synthetic construct
  misc_feature 1..22: note = IMP R'
  source 1..22: mol_type = other DNA
aagcttctaaatttgcgtcacc

SEQ ID NO: 48 - DNA, 24 bp, synthetic construct
  misc_feature 1..24: note = IMP-TYE705
  source 1..24: mol_type = other DNA
tttgttaaaccggacggtcttggt

SEQ ID NO: 49 - DNA, 201 bp, synthetic construct
  misc_feature 1..201: note = MOX
  source 1..201: mol_type = other DNA
aaccgggagagcggggccagcgtcagcgagcagaccctgttcgagataggatccgtgagcaagaccctgactgcgaccctgggggcctatgcggtggtcaagggagcgatgcagctggatgacaaggcgagccggcacgcgccctggctcaagggatccgtctttgacagcatcaccatgggggagcttgccacctacagc

SEQ ID NO: 50 - DNA, 240 bp, synthetic construct
  misc_feature 1..240: note = FOX
  source 1..240: mol_type = other DNA
ggggatggcggtcgccgtgctgaaagatggcaaggcccactatttcaactatggggttgccaaccgcgagagtggtcagcgcgtcagcgagcagaccctgttcgagattggctcggtcagcaagaccctgaccgcgaccctcggtgcctatgctgcggtcaaggggggctttgagctggatgacaaggtgagccagcacgccccctggctcaaaggttccgcctttgatggtgtgaccat

SEQ ID NO: 51 - DNA, 220 bp, synthetic construct
  misc_feature 1..220: note = EBC
  source 1..220: mol_type = other DNA
ggaccgttacgccgctgatgaaagcgcaggccattccgggtatggcggtggcggtgatttatgagggtcagccgcactacttcaccttcggtaaagccgatgttgcggcgaacaaacctgtcactccacaaaccttgttcgaactgggttctataagtaaaaccttcaccggcgtactcggtggcgatgccattgctcgcggtgaaatatcgctgggcga

SEQ ID NO: 52 - DNA, 220 bp, synthetic construct
  misc_feature 1..220: note = DHA
  source 1..220: mol_type = other DNA
gactgcacggatcctggcaccgctggggttatctcacacctttattactgtgccggaaagtgcgcaaagccagtatgcgtacggttatgagaacaaaaaaccggtccgcgtgtcgccgggacagcttgatgcggaatcttacggcgtggaatccgcctcaaaagatatgctgcgctgggcggaaatgaatatggagccgtcacgggccggtaatgcggat

SEQ ID NO: 53 - DNA, 260 bp, synthetic construct
  misc_feature 1..260: note = CMY
  source 1..260: mol_type = other DNA
gcctgtacacgtttctccgggacaacttgacgccgaagcctatggcgtgaaatccagcgttattgatatggcccgctgggttcaggtcaacatggacgccagccgcgttcaggagaaaacgctccagcagggcattgcgcttgcgcagtctcgctactggcgtattggcgatatgtaccagggattaggctgggagatgctgaactggccgctgaaagctgattcgatcatcaacggtagcgacagcaaagtggcattgg

SEQ ID NO: 54 - DNA, 150 bp, synthetic construct
  misc_feature 1..150: note = ACC
  source 1..150: mol_type = other DNA
gagagcaaaattaaagacaccgttgatgacctgatccagccgctgatgcagaagaataatattcccggtatgtcggtcgcagtgaccgtcaacggtaaaaactacatttataactatgggttagcggcaaaacagcctcagcagccggtt

SEQ ID NO: 55 - DNA, 270 bp, synthetic construct
  misc_feature 1..270: note = IC
  source 1..270: mol_type = other DNA
agcttgttggtggggtaacggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccg

SEQ ID NO: 56 - DNA, 321 bp, synthetic construct
  misc_feature 1..321: note = CTX-M-14
  source 1..321: mol_type = other DNA
cgtttcgtctggatcgcactgaacctacgctgaataccgccattcccggcgacccgagagacaccaccacgccgcgggcgatggcgcagacgttgcgtcagcttacgctgggtcatgcgctgggcgaaacccagcgggcgcagttggtgacgtggctcaaaggcaatacgaccggcgcagccagcattcgggccggcttaccgacgtcgtggactgtgggtgataagaccggcagcggcgactacggcaccaccaatgatattgcggtgatctggccgcagggtcgtgcgccgctggttctggtgacctattttacccagc

SEQ ID NO: 57 - DNA, 121 bp, synthetic construct
  misc_feature 1..121: note = CTX-M-15
  source 1..121: mol_type = other DNA
ccgtcacgctgttgttaggaagtgtgccgctgtatgcgcaaacggcggacgtacagcaaaaacttgccgaattagagcggcagtcgggaggcagactgggtgtggcattgattaacacagc

SEQ ID NO: 58 - DNA, 553 bp, synthetic construct
  misc_feature 1..553: note = OXA
  source 1..553: mol_type = other DNA
aatcacagggcgtagttgtgctctggaatgagaataagcagcaaggatttaccaataatcttaaacgggcgaaccaagcatttttacccgcatctacctttaaaattcccaatagcttgatcgccctcgatttgggcgtggttaaggatgaacaccaagtctttaagtgggatggacagacgcgcgatatcgccacttggaatcgcgatcataatctaatcaccgcgatgaaatattcagttgtgcctgtttatcaagaatttgcccgccaaattggcgaggcacgtatgagcaagatgctacatgctttcgattatggtaatgaggacatttcgggcaatgtagacagtttctggctcgacggtggtattcgaatttcggccacggagcaaatcagctttttaagaaagctgtatcacaataagttacacgtatcggagcgcagccagcgtattgtcaaacaagccatgctgaccgaagccaatggtgactaattattcgggctaaaactggatactcgactagaatcgaacctaagattggctggctgggt

SEQ ID NO: 59 - DNA, 270 bp, synthetic construct
  misc_feature 1..270: note = IC
  source 1..270: mol_type = other DNA
cggagttagccggtgcttcttctgcgggtaacgtcaatgagcaaaggtattaactttactcccttcctccccgctgaaagtactttacaacccgaaggccttcttcatacacgcggcatggctgcatcaggcttgcgcccattgtgcaatattccccactgctgcctcccgtaggagtctggaccgtgtctcagttccagtgtggctggtcatcctctcagaccagctagggatcgtcgccttggtgagccgttaccccaccaacaagct

SEQ ID NO: 60 - DNA, 260 bp, synthetic construct; identical to SEQ ID NO: 53
  misc_feature 1..260: note = CMY
  source 1..260: mol_type = other DNA

SEQ ID NO: 61 - DNA, 263 bp, synthetic construct
  misc_feature 1..263: note = NDM
  source 1..263: mol_type = other DNA
ggcgaaagtcaggctgtgttgcgccgcaaccatcccctcttgcggggcaagctggttcgacaacgcattggcataagtcgcaatccccgccgcatgcagcgcgtccataccgcccatcttgtcctgatgcgcgtgagtcaccaccgccagcgcgaccggcaggttgatctcctgcttgatccagttgaggatctgggcggtctggtcatcggtccaggcggtatcgaccaccagcacgcggccgccatccctgacgatcaaac

SEQ ID NO: 62 - DNA, 209 bp, synthetic construct
  misc_feature 1..209: note = KPC
  source 1..209: mol_type = other DNA
gtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagg

SEQ ID NO: 63 - DNA, 292 bp, synthetic construct
  misc_feature 1..292: note = VIM
  source 1..292: mol_type = other DNA
ccattcagccagatcggcatcggccacgttccccgcagacgtgcgtgacaactcatgaatcgcacaaccaccatagagcacactcgcagacgggacgtacacaactaagttgtcggtcgaatgcgcagcaccaggatagaagagttctactggaccgaagcgcactgcgtccccgctcgagtccttctagagagtgcgtgggaatctcgttcccctctacctcggctagccggcgtgtcgacggtgatgcgtacgttgccaccccagccgcccgaaggacatcaacgccgcc

SEQ ID NO: 64 - DNA, 220 bp, synthetic construct
  misc_feature 1..220: note = DHA
  source 1..220: mol_type = other DNA
gactgcacggatcctggcaccgctggggttatctcacacctttattactgtgccggaaagtgcgcaaagccagtatgcgtacggttatgagaacaaaaaaccggtccgcgtgtcgccgggacagcttgatgcggaatcttacggcgtgaaatccgcctcaaaagatatgctgcgctgggcggaaatgaatatggagccgtcacgggccggtaatgcggat

SEQ ID NO: 65 - DNA, 270 bp, synthetic construct; identical to SEQ ID NO: 59
  misc_feature 1..270: note = IC
  source 1..270: mol_type = other DNA

SEQ ID NO: 66 - DNA, 177 bp, synthetic construct
  misc_feature 1..177: note = IMP
  source 1..177: mol_type = other DNA
gcggagttagttattggctagttaaaaataaaattgaagttttttatcccggcccggggcacactcaagataacgtagtggtttggttacctgaaaagaaaattttattcggtggttgttttgttaaaccggacggtcttgrvgtaatttgggtgacgcaaatttagaagcttggcc

SEQ ID NO: 67 - DNA, 21 bp, synthetic construct
  misc_feature 1..21: note = PRIMER/PROBE
  source 1..21: mol_type = other DNA
tggccagaactgacaggcaaa

SEQ ID NO: 68 - DNA, 21 bp, synthetic construct
  misc_feature 1..21: note = PRIMER/PROBE
  source 1..21: mol_type = other DNA
tttctcctgaacgtggctggc

SEQ ID NO: 69 - DNA, 24 bp, synthetic construct
  misc_feature 1..24: note = PRIMER/PROBE
  source 1..24: mol_type = other DNA
acgctaactccagcattggtctgt

SEQ ID NO: 70 - DNA, 19 bp, synthetic construct
  misc_feature 1..19: note = PRIMER/PROBE
  source 1..19: mol_type = other DNA
ccgtcacgctgttgttagg

SEQ ID NO: 71 - DNA, 22 bp, synthetic construct
  misc_feature 1..22: note = PRIMER/PROBE
  source 1..22: mol_type = other DNA
gctgtgttaatcaatgccacac

SEQ ID NO: 72 - DNA, 24 bp, synthetic construct
  misc_feature 1..24: note = PRIMER/PROBE
  source 1..24: mol_type = other DNA
aacttgccgaattagagcrgcagt

SEQ ID NO: 73 - DNA, 19 bp, synthetic construct
  misc_feature 1..19: note = PRIMER/PROBE
  source 1..19: mol_type = other DNA
cgtttcgtctggatcgcac

SEQ ID NO: 74 - DNA, 20 bp, synthetic construct
  misc_feature 1..20: note = PRIMER/PROBE
  source 1..20: mol_type = other DNA
gctgggtaaaataggtcacc

SEQ ID NO: 75 - DNA, 24 bp, synthetic construct
  misc_feature 1..24: note = PRIMER/PROBE
  source 1..24: mol_type = other DNA
tatcattggtggtgccgtagtcgc

SEQ ID NO: 76 - DNA, 20 bp, synthetic construct
  misc_feature 1..20: note = PRIMER/PROBE
  source 1..20: mol_type = other DNA
gagaggatgaycagccacac

SEQ ID NO: 77 - DNA, 20 bp, synthetic construct
  misc_feature 1..20: note = PRIMER/PROBE
  source 1..20: mol_type = other DNA
cgcccattgtscaatattcc

SEQ ID NO: 78 - DNA, 24 bp, synthetic construct
  misc_feature 1..24: note = PRIMER/PROBE
  source 1..24: mol_type = other DNA
tgagacacggtccagactcctacg

SEQ ID NO: 79 - DNA, 20 bp, synthetic construct
  misc_feature 1..20: note = PRIMER/PROBE
  source 1..20: mol_type = other DNA
aatcacagggcgtagttgtg

SEQ ID NO: 80 - DNA, 20 bp, synthetic construct
  misc_feature 1..20: note = PRIMER/PROBE
  source 1..20: mol_type = other DNA
acccaccagccaatcttagg

SEQ ID NO: 81 - DNA, 24 bp, synthetic construct
  misc_feature 1..24: note = PRIMER/PROBE
  source 1..24: mol_type = other DNA
tagcttgatcgccctcgatttggg

SEQ ID NO: 82 - DNA, 21 bp, synthetic construct
  misc_feature 1..21: note = PRIMER/PROBE
  source 1..21: mol_type = other DNA
gcggagttaactattggctag

SEQ ID NO: 83 - DNA, 21 bp, synthetic construct
  misc_feature 1..21: note = PRIMER/PROBE
  source 1..21: mol_type = other DNA
ggccaagcttctatatttgcg

SEQ ID NO: 84 - DNA, 23 bp, synthetic construct
  misc_feature 1..23: note = PRIMER/PROBE
  source 1..23: mol_type = other DNA
ttrttyggtggttgytttrttaa

SEQ ID NO: 85 - DNA, 21 bp, synthetic construct
  misc_feature 1..21: note = PRIMER/PROBE
  source 1..21: mol_type = other DNA
gcggagttarytattggctag

SEQ ID NO: 86 - DNA, 21 bp, synthetic construct
  misc_feature 1..21: note = PRIMER/PROBE
  source 1..21: mol_type = other DNA
ggccaagcytctawatttgcg

SEQ ID NO: 87 - DNA, 24 bp, synthetic construct
  misc_feature 1..24: note = PRIMER/PROBE
  source 1..24: mol_type = other DNA
ccggacggtcttggtaatttgggt

SEQ ID NO: 88 - DNA, 24 bp, synthetic construct
  misc_feature 1..24: note = PRIMER/PROBE
  source 1..24: mol_type = other DNA
ccgtacggtttaggcaatttgggt

SEQ ID NO: 89 - DNA, 20 bp, synthetic construct
  misc_feature 1..20: note = PRIMER/PROBE
  source 1..20: mol_type = other DNA
ggcggcgttgatgtccttcg

SEQ ID NO: 90 - DNA, 21 bp, synthetic construct
  misc_feature 1..21: note = PRIMER/PROBE
  source 1..21: mol_type = other DNA
ccattcagccagatcggcatc

SEQ ID NO: 91 - DNA, 23 bp, synthetic construct
  misc_feature 1..23: note = PRIMER/PROBE
  source 1..23: mol_type = other DNA
agctcttctatcctggtgctgcg

SEQ ID NO: 92 - DNA, 22 bp, synthetic construct
  misc_feature 1..22: note = PRIMER/PROBE
  source 1..22: mol_type = other DNA
aactttcacaggtgtgctgggt

SEQ ID NO: 93 - DNA, 21 bp, synthetic construct
  misc_feature 1..21: note = PRIMER/PROBE
  source 1..21: mol_type = other DNA
ccgtacgcatactggctttgc

SEQ ID NO: 94 - DNA, 24 bp, synthetic construct
  misc_feature 1..24: note = PRIMER/PROBE
  source 1..24: mol_type = other DNA
aaaccgggcgatatgcgtctgtat

SEQ ID NO: 95 - DNA, 20 bp, synthetic construct
  misc_feature 1..20: note = PRIMER/PROBE
  source 1..20: mol_type = other DNA
gtatcgccgtctagttctgc

SEQ ID NO: 96 - DNA, 22 bp, synthetic construct
  misc_feature 1..22: note = PRIMER/PROBE
  source 1..22: mol_type = other DNA
ccttgaatgagctgcacagtgg

SEQ ID NO: 97 - DNA, 23 bp, synthetic construct
  misc_feature 1..23: note = PRIMER/PROBE
  source 1..23: mol_type = other DNA
tcgtcgcggaaccattcgctaaa

SEQ ID NO: 98 - DNA, 20 bp, synthetic construct
  misc_feature 1..20: note = PRIMER/PROBE
  source 1..20: mol_type = other DNA
gtttgatcgtcagggatggc

SEQ ID NO: 99 - DNA, 18 bp, synthetic construct
  misc_feature 1..18: note = PRIMER/PROBE
  source 1..18: mol_type = other DNA
ggcgaaagtcaggctgtg

SEQ ID NO: 100 - DNA, 24 bp, synthetic construct
  misc_feature 1..24: note = PRIMER/PROBE
  source 1..24: mol_type = other DNA
catcaggacaagatgggcggtatg

SEQ ID NO: 101 - DNA, 21 bp, synthetic construct
  misc_feature 1..21: note = PRIMER/PROBE
  source 1..21: mol_type = other DNA
gctgctcaaggagcacaggat

SEQ ID NO: 102 - DNA, 22 bp, synthetic construct
  misc_feature 1..22: note = PRIMER/PROBE
  source 1..22: mol_type = other DNA
cacattgacataggtgtggtgc

SEQ ID NO: 103 - DNA, 24 bp, synthetic construct
  misc_feature 1..24: note = PRIMER/PROBE
  source 1..24: mol_type = other DNA
aggatggcaaggcccactatttca

SEQ ID NO: 104 - DNA, 21 bp, synthetic construct
  misc_feature 1..21: note = PRIMER/PROBES
  source 1..21: mol_type = other DNA
aacagcctcagcagccggtta

SEQ ID NO: 105 - DNA, 21 bp, synthetic construct
  misc_feature 1..21: note = PRIMER/PROBE
  source 1..21: mol_type = other DNA
ttcgccgcaatcatccctagc

SEQ ID NO: 106 - DNA, 24 bp, synthetic construct
  misc_feature 1..24: note = PRIMER/PROBE
  source 1..24: mol_type = other DNA
agccattacgttccagagttgcgt

SEQ ID NO: 107 - DNA, 21 bp, synthetic construct
  misc_feature 1..21: note = PRIMER/PROBE
  source 1..21: mol_type = other DNA
gccgaggcttacgggatcaag

SEQ ID NO: 108 - DNA, 21 bp, synthetic construct
  misc_feature 1..21: note = PRIMER/PROBE
  source 1..21: mol_type = other DNA
caaagcgcgtaaccggattgg

SEQ ID NO: 109 - DNA, 24 bp, synthetic construct
  misc_feature 1..24: note = PRIMER/PROBE
  source 1..24: mol_type = other DNA
tctgctgaagtttrycgaggcmaa

SEQ ID NO: 110 - DNA, 22 bp, synthetic construct
  misc_feature 1..22: note = PRIMER/PROBE
  source 1..22: mol_type = other DNA
aactttcacaggtgtgctgggt

SEQ ID NO: 111 - DNA, 21 bp, synthetic construct
  misc_feature 1..21: note = PRIMER/PROBE
  source 1..21: mol_type = other DNA
ccgtacgcatactggctttgc

SEQ ID NO: 112 - DNA, 24 bp, synthetic construct
  misc_feature 1..24: note = PRIMER/PROBE
  source 1..24: mol_type = other DNA
aaaccgggcgatatgcgtctgtat

SEQ ID NO: 113 - DNA, 29 bp, synthetic construct
  misc_feature 1..29: note = PRIMER/PROBE
  source 1..29: mol_type = other DNA
ctgggttctataagtaaaaccttcaccgg

SEQ ID NO: 114 - DNA, 21 bp, synthetic construct
  misc_feature 1..21: note = PRIMER/PROBE
  source 1..21: mol_type = other DNA
cttccactgcggctgccagtt

SEQ ID NO: 115 - DNA, 23 bp, synthetic construct
  misc_feature 1..23: note = PRIMER/PROBE
  source 1..23: mol_type = other DNA
gatgccattgcycgsggtgaaat

SEQ ID NO: 116 - DNA, 23 bp, synthetic construct
  misc_feature 1..23: note = PRIMER/PROBE
  source 1..23: mol_type = other DNA
ccgaagcctatggcgtgaaatcc

SEQ ID NO: 117 - DNA, 19 bp, synthetic construct
  misc_feature 1..19: note = PRIMER/PROBE
  source 1..19: mol_type = other DNA
gcaatgccctgctggagcg

SEQ ID NO: 118 - DNA, 20 bp, synthetic construct
  misc_feature 1..20: note = PRIMER/PROBE
  source 1..20: mol_type = other DNA
atgttggcctgaacccagcg

SEQ ID NO: 119 - DNA, 24 bp, synthetic construct
  misc_feature 1..24: note = PRIMER/PROBE
  source 1..24: mol_type = other DNA
agcacatacagaatatgtccctgc

SEQ ID NO: 120 - DNA, 25 bp, synthetic construct
  misc_feature 1..25: note = PRIMER/PROBE
  source 1..25: mol_type = other DNA
acctgttaaccaacctacttgaggg

SEQ ID NO: 121 - DNA, 24 bp, synthetic construct
  misc_feature 1..24: note = PRIMER/PROBE
  source 1..24: mol_type = other DNA
ttgcaagacggactggcttagacc

SEQ ID NO: 122 - DNA, 20 bp, synthetic construct
  misc_feature 1..20: note = PRIMER/PROBE
  source 1..20: mol_type = other DNA
cctgatcggattggagaacc

SEQ ID NO: 123 - DNA, 23 bp, synthetic construct
  misc_feature 1..23: note = PRIMER/PROBE
  source 1..23: mol_type = other DNA
ctacctcttgaataggcgtaacc

SEQ ID NO: 124 - DNA, 24 bp, synthetic construct
  misc_feature 1..24: note = PRIMER/PROBE
  source 1..24: mol_type = other DNA
acgtcgcgcaagttcctgatagac

SEQ ID NO: 125 - DNA, 25 bp, synthetic construct
  misc_feature 1..25: note = PRIMER/PROBE
  source 1..25: mol_type = other DNA
tagtgactgctaatccaaatcacag

SEQ ID NO: 126 - DNA, 25 bp, synthetic construct
  misc_feature 1..25: note = PRIMER/PROBE
  source 1..25: mol_type = other DNA
gcacgagcaagatcattaccatagc

SEQ ID NO: 127 - DNA, 27 bp, synthetic construct
  misc_feature 1..27: note = PRIMER/PROBE
  source 1..27: mol_type = other DNA
agttatccaacaaggccaaactcaaca

SEQ ID NO: 128 - DNA, 20 bp, synthetic construct
  misc_feature 1..20: note = PRIMER/PROBE
  source 1..20: mol_type = other DNA
aatcacagggcgtagttgtg

SEQ ID NO: 129 - DNA, 20 bp, synthetic construct
  misc_feature 1..20: note = PRIMER/PROBE
  source 1..20: mol_type = other DNA
acccaccagccaatcttagg

SEQ ID NO: 130 - DNA, 24 bp, synthetic construct
  misc_feature 1..24: note = PRIMER/PROBE
  source 1..24: mol_type = other DNA
tagcttgatcgccctcgatttggg

SEQ ID NO: 131 - DNA, 18 bp, synthetic construct
  misc_feature 1..18: note = PRIMER/PROBE
  source 1..18: mol_type = other DNA
gtgggatggaaagccacg

SEQ ID NO: 132 - DNA, 18 bp, synthetic construct
  misc_feature 1..18: note = PRIMER/PROBE
  source 1..18: mol_type = other DNA
cacttgcgggtctacagc

SEQ ID NO: 133 - DNA, 24 bp, synthetic construct
  misc_feature 1..24: note = PRIMER/PROBE
  source 1..24: mol_type = other DNA
ttactttgggcgaagccatgcaag

SEQ ID NO: 134 - DNA, 21 bp, synthetic construct
  misc_feature 1..21: note = PRIMER/PROBE
  source 1..21: mol_type = other DNA
cacctatggtaatgctcttgc

SEQ ID NO: 135 - DNA, 20 bp, synthetic construct
  misc_feature 1..20: note = PRIMER/PROBE
  source 1..20: mol_type = other DNA
ctggaactgctgacaatgcc

SEQ ID NO: 136 - DNA, 30 bp, synthetic construct
  misc_feature 1..30: note = PRIMER/PROBE
  source 1..30: mol_type = other DNA
tgggagaaagatatgactttaggtgaggca

SEQ ID NO: 137 - DNA, 18 bp, synthetic construct
  misc_feature 1..18: note = PRIMER/PROBES
  source 1..18: mol_type = other DNA
ccgtgtatgttcagctat

SEQ ID NO: 138 - DNA, 18 bp, synthetic construct
  misc_feature 1..18: note = PRIMER/PROBE
  source 1..18: mol_type = other DNA
cttatccatcacgccttt

SEQ ID NO: 139 - DNA, 26 bp, synthetic construct
  misc_feature 1..26: note = PRIMER/PROBES
  source 1..26: mol_type = other DNA
tatgatgtcgataccgccaaatacca

SEQ ID NO: 140 - DNA, 18 bp, synthetic construct
  misc_feature 1..18: note = PRIMER/PROBE
  source 1..18: mol_type = other DNA
ctgtatgtcagcgatcat

SEQ ID NO: 141 - DNA, 19 bp, synthetic construct
  misc_feature 1..19: note = PRIMER/PROBE
  source 1..19: mol_type = other DNA
gatgccagtttgcttatcc

SEQ ID NO: 142 - DNA, 25 bp, synthetic construct
  misc_feature 1..25: note = PRIMER/PROBE
  source 1..25: mol_type = other DNA
aagtctgggtgagaacggtgtctat

SEQ ID NO: 143 - DNA, 19 bp, synthetic construct
  misc_feature 1..19: note = PRIMER/PROBE
  source 1..19: mol_type = other DNA
cagtcagtatgcgagtttc

SEQ ID NO: 144 - DNA, 18 bp, synthetic construct
  misc_feature 1..18: note = PRIMER/PROBE
  source 1..18: mol_type = other DNA
aaaattcgccaagccatc

SEQ ID NO: 145 - DNA, 25 bp, synthetic construct
  misc_feature 1..25: note = PRIMER/PROBE
  source 1..25: mol_type = other DNA
tgcataagccagtgcgtttttatat

SEQ ID NO: 146 - DNA, 18 bp, synthetic construct
  misc_feature 1..18: note = PRIMER/PROBE
  source 1..18: mol_type = other DNA
agatcagttgggtgcacg

SEQ ID NO: 147 - DNA, 20 bp, synthetic construct
  misc_feature 1..20: note = PRIMER/PROBE
  source 1..20: mol_type = other DNA
tgcttaatcagtgaggcacc

SEQ ID NO: 148 - DNA, 24 bp, synthetic construct
  misc_feature 1..24: note = PRIMER/PROBE
  source 1..24: mol_type = other DNA
atgaagccataccaaacgacgagc

SEQ ID NO: 149 - DNA, 20 bp, synthetic construct
  misc_feature 1..20: note = PRIMER/PROBE
  source 1..20: mol_type = other DNA
ctggagcgaaagatccacta

SEQ ID NO: 150 - DNA, 18 bp, synthetic construct
  misc_feature 1..18: note = PRIMER/PROBE
  source 1..18: mol_type = other DNA
atcgtccaccatccactg

SEQ ID NO: 151 - DNA, 22 bp, synthetic construct
  misc_feature 1..22: note = PRIMER/PROBE
  source 1..22: mol_type = other DNA
ccagatcggcgacaacgtcacc

SEQ ID NO: 152 - DNA, 21 bp, synthetic construct
  misc_feature 1..21: note = PRIMER/PROBE
  source 1..21: mol_type = other DNA
tggccagaactgacaggcaaa

SEQ ID NO: 153 - DNA, 21 bp, synthetic construct
  misc_feature 1..21: note = PRIMER/PROBE
  source 1..21: mol_type = other DNA
tttctcctgaacgtggctggc

SEQ ID NO: 154 - DNA, 24 bp, synthetic construct
  misc_feature 1..24: note = PRIMER/PROBE
  source 1..24: mol_type = other DNA
acgctaactccagcattggtctgt

SEQ ID NO: 155 - DNA, 19 bp, synthetic construct
  misc_feature 1..19: note = PRIMER/PROBE
  source 1..19: mol_type = other DNA
ccgtcacgctgttgttagg

SEQ ID NO: 156 - DNA, 22 bp, synthetic construct
  misc_feature 1..22: note = PRIMER/PROBE
  source 1..22: mol_type = other DNA
gctgtgttaatcaatgccacac

SEQ ID NO: 157 - DNA, 24 bp, synthetic construct
  misc_feature 1..24: note = PRIMER/PROBE
  source 1..24: mol_type = other DNA
aacttgccgaattagagcrgcagt

SEQ ID NO: 158 - DNA, 19 bp, synthetic construct
  misc_feature 1..19: note = PRIMER/PROBE
  source 1..19: mol_type = other DNA
cgtttcgtctggatcgcac

SEQ ID NO: 159 - DNA, 20 bp, synthetic construct
  misc_feature 1..20: note = PRIMER/PROBE
  source 1..20: mol_type = other DNA
gctgggtaaaataggtcacc

SEQ ID NO: 160 - DNA, 24 bp, synthetic construct
  misc_feature 1..24: note = PRIMER/PROBE
  source 1..24: mol_type = other DNA
tatcattggtggtgccgtagtcgc

SEQ ID NO: 161 - DNA, 20 bp, synthetic construct
  misc_feature 1..20: note = PRIMER/PROBE
  source 1..20: mol_type = other DNA
gagaggatgaycagccacac

SEQ ID NO: 162 - DNA, 20 bp, synthetic construct
  misc_feature 1..20: note = PRIMER/PROBE
  source 1..20: mol_type = other DNA
cgcccattgtscaatattcc

SEQ ID NO: 163 - DNA, 24 bp, synthetic construct
  misc_feature 1..24: note = PRIMER/PROBE
  source 1..24: mol_type = other DNA
tgagacacggtccagactcctacg

SEQ ID NO: 164 - DNA, 20 bp, synthetic construct
  misc_feature 1..20: note = PRIMER/PROBE
  source 1..20: mol_type = other DNA
aatcacagggcgtagttgtg

SEQ ID NO: 165 - DNA, 20 bp, synthetic construct
  misc_feature 1..20: note = PRIMER/PROBE
  source 1..20: mol_type = other DNA
acccaccagccaatcttagg

SEQ ID NO: 166 - DNA, 24 bp, synthetic construct
  misc_feature 1..24: note = PRIMER/PROBE
  source 1..24: mol_type = other DNA
tagcttgatcgccctcgatttggg

SEQ ID NO: 167 - DNA, 21 bp, synthetic construct
  misc_feature 1..21: note = PRIMER/PROBE
  source 1..21: mol_type = other DNA
gcggagttaactattggctag

SEQ ID NO: 168 - DNA, 21 bp, synthetic construct
  misc_feature 1..21: note = PRIMER/PROBE
  source 1..21: mol_type = other DNA
ggccaagcttctatatttgcg

SEQ ID NO: 169 - DNA, 23 bp, synthetic construct
  misc_feature 1..23: note = PRIMER/PROBE
  source 1..23: mol_type = other DNA
ttrttyggtggttgytttrttaa

SEQ ID NO: 170 - DNA, 21 bp, synthetic construct
  misc_feature 1..21: note = PRIMER/PROBE
  source 1..21: mol_type = other DNA
gcggagttarytattggctag

SEQ ID NO: 171 - DNA, 21 bp, synthetic construct
  misc_feature 1..21: note = PRIMER/PROBE
  source 1..21: mol_type = other DNA
ggccaagcytctawatttgcg

SEQ ID NO: 172 - DNA, 24 bp, synthetic construct
  misc_feature 1..24: note = PRIMER/PROBE
  source 1..24: mol_type = other DNA
ccggacggtcttggtaatttgggt

SEQ ID NO: 173 - DNA, 24 bp, synthetic construct
  misc_feature 1..24: note = PRIMER/PROBE
  source 1..24: mol_type = other DNA
ccgtacggtttaggcaatttgggt

SEQ ID NO: 174 - DNA, 20 bp, synthetic construct
  misc_feature 1..20: note = PRIMER/PROBE
  source 1..20: mol_type = other DNA
ggcggcgttgatgtccttcg

SEQ ID NO: 175 - DNA, 21 bp, synthetic construct
  misc_feature 1..21: note = PRIMER/PROBE
  source 1..21: mol_type = other DNA
ccattcagccagatcggcatc

SEQ ID NO: 176 - DNA, 23 bp, synthetic construct
  misc_feature 1..23: note = PRIMER/PROBE
  source 1..23: mol_type = other DNA
agctcttctatcctggtgctgcg

SEQ ID NO: 177 - DNA, 20 bp, synthetic construct
  misc_feature 1..20: note = PRIMER/PROBE
  source 1..20: mol_type = other DNA
gagaggatgaycagccacac

SEQ ID NO: 178 - DNA, 20 bp, synthetic construct
  misc_feature 1..20: note = PRIMER/PROBE
  source 1..20: mol_type = other DNA
cgcccattgtscaatattcc

SEQ ID NO: 179 - DNA, 24 bp, synthetic construct
  misc_feature 1..24: note = PRIMER/PROBE
  source 1..24: mol_type = other DNA
tgagacacggtccagactcctacg

SEQ ID NO: 180 - DNA, 22 bp, synthetic construct
  misc_feature 1..22: note = PRIMER/PROBE
  source 1..22: mol_type = other DNA
aactttcacaggtgtgctgggt

SEQ ID NO: 181 - DNA, 21 bp, synthetic construct
  misc_feature 1..21: note = PRIMER/PROBE
  source 1..21: mol_type = other DNA
ccgtacgcatactggctttgc

SEQ ID NO: 182 - DNA, 24 bp, synthetic construct
  misc_feature 1..24: note = PRIMER/PROBE
  source 1..24: mol_type = other DNA
aaaccgggcgatatgcgtctgtat

SEQ ID NO: 183 - DNA, 20 bp, synthetic construct
  misc_feature 1..20: note = PRIMER/PROBE
  source 1..20: mol_type = other DNA
gtatcgccgtctagttctgc

SEQ ID NO: 184 - DNA, 22 bp, synthetic construct
  misc_feature 1..22: note = PRIMER/PROBE
  source 1..22: mol_type = other DNA
ccttgaatgagctgcacagtgg

SEQ ID NO: 185 - DNA, 23 bp, synthetic construct
  misc_feature 1..23: note = PRIMER/PROBE
  source 1..23: mol_type = other DNA
tcgtcgcggaaccattcgctaaa

SEQ ID NO: 186 - DNA, 20 bp, synthetic construct
  misc_feature 1..20: note = PRIMER/PROBE
  source 1..20: mol_type = other DNA
gtttgatcgtcagggatggc

SEQ ID NO: 187 - DNA, 18 bp, synthetic construct
  misc_feature 1..18: note = PRIMER/PROBE
  source 1..18: mol_type = other DNA
ggcgaaagtcaggctgtg

SEQ ID NO: 188 - DNA, 24 bp, synthetic construct
  misc_feature 1..24: note = PRIMER/PROBE
  source 1..24: mol_type = other DNA
catcaggacaagatgggcggtatg

SEQ ID NO: 189 - DNA, 20 bp, synthetic construct
  misc_feature 1..20: note = PRIMER/PROBE
  source 1..20: mol_type = other DNA
gagaggatgaycagccacac

SEQ ID NO: 190 - DNA, 20 bp, synthetic construct
  misc_feature 1..20: note = PRIMER/PROBE
  source 1..20: mol_type = other DNA
cgcccattgtscaatattcc

SEQ ID NO: 191 - DNA, 24 bp, synthetic construct
  misc_feature 1..24: note = PRIMER/PROBE
  source 1..24: mol_type = other DNA
tgagacacggtccagactcctacg

SEQ ID NO: 192 - DNA, 21 bp, synthetic construct
  misc_feature 1..21: note = PRIMER/PROBE
  source 1..21: mol_type = other DNA
gctgctcaaggagcacaggat

SEQ ID NO: 193 - DNA, 22 bp, synthetic construct
  misc_feature 1..22: note = PRIMER/PROBE
  source 1..22: mol_type = other DNA
cacattgacataggtgtggtgc

SEQ ID NO: 194 - DNA, 24 bp, synthetic construct
  misc_feature 1..24: note = PRIMER/PROBE
  source 1..24: mol_type = other DNA
aggatggcaaggcccactatttca

SEQ ID NO: 195 - DNA, 21 bp, synthetic construct
  misc_feature 1..21: note = PRIMER/PROBE
  source 1..21: mol_type = other DNA
aacagcctcagcagccggtta

SEQ ID NO: 196 - DNA, 21 bp, synthetic construct
  misc_feature 1..21: note = PRIMER/PROBE
  source 1..21: mol_type = other DNA
ttcgccgcaatcatccctagc

SEQ ID NO: 197 - DNA, 24 bp, synthetic construct
  misc_feature 1..24: note = PRIMER/PROBE
  source 1..24: mol_type = other DNA
agccattacgttccagagttgcgt

SEQ ID NO: 198 - DNA, 21 bp, synthetic construct
  misc_feature 1..21: note = PRIMER/PROBE
  source 1..21: mol_type = other DNA
gccgaggcttacgggatcaag

SEQ ID NO: 199 - DNA, 21 bp, synthetic construct
  misc_feature 1..21: note = PRIMER/PROBE
  source 1..21: mol_type = other DNA
caaagcgcgtaaccggattgg

SEQ ID NO: 200 - DNA, 24 bp, synthetic construct
  misc_feature 1..24: note = PRIMER/PROBE
  source 1..24: mol_type = other DNA
tctgctgaagtttrycgaggcmaa

SEQ ID NO: 201 - DNA, 20 bp, synthetic construct
  misc_feature 1..20: note = PRIMER/PROBE
  source 1..20: mol_type = other DNA
gagaggatgaycagccacac

SEQ ID NO: 202 - DNA, 20 bp, synthetic construct
  misc_feature 1..20: note = PRIMER/PROBE
  source 1..20: mol_type = other DNA
cgcccattgtscaatattcc

SEQ ID NO: 203 - DNA, 24 bp, synthetic construct
  misc_feature 1..24: note = PRIMER/PROBE
  source 1..24: mol_type = other DNA
tgagacacggtccagactcctacg

SEQ ID NO: 204 - DNA, 22 bp, synthetic construct
  misc_feature 1..22: note = PRIMER/PROBE
  source 1..22: mol_type = other DNA
aactttcacaggtgtgctgggt

SEQ ID NO: 205 - DNA, 21 bp, synthetic construct
  misc_feature 1..21: note = PRIMER/PROBE
  source 1..21: mol_type = other DNA
ccgtacgcatactggctttgc

SEQ ID NO: 206 - DNA, 24 bp, synthetic construct
  misc_feature 1..24: note = PRIMER/PROBE
  source 1..24: mol_type = other DNA
aaaccgggcgatatgcgtctgtat

SEQ ID NO: 207 - DNA, 29 bp, synthetic construct
  misc_feature 1..29: note = PRIMER/PROBE
  source 1..29: mol_type = other DNA
ctgggttctataagtaaaaccttcaccgg

SEQ ID NO: 208 - DNA, 21 bp, synthetic construct
  misc_feature 1..21: note = PRIMER/PROBE
  source 1..21: mol_type = other DNA
cttccactgcggctgccagtt

SEQ ID NO: 209 - DNA, 23 bp, synthetic construct
  misc_feature 1..23: note = PRIMER/PROBE
  source 1..23: mol_type = other DNA
gatgccattgcycgsggtgaaat

SEQ ID NO: 210 - DNA, 23 bp, synthetic construct
  misc_feature 1..23: note = PRIMER/PROBE
  source 1..23: mol_type = other DNA
ccgaagcctatggcgtgaaatcc

SEQ ID NO: 211 - DNA, 19 bp, synthetic construct
  misc_feature 1..19: note = PRIMER/PROBE
  source 1..19: mol_type = other DNA
gcaatgccctgctggagcg

SEQ ID NO: 212 - DNA, 20 bp, synthetic construct
  misc_feature 1..20: note = PRIMER/PROBE
  source 1..20: mol_type = other DNA
atgttggcctgaacccagcg

SEQ ID NO: 213 - DNA, 20 bp, synthetic construct
  misc_feature 1..20: note = PRIMER/PROBE
  source 1..20: mol_type = other DNA
gagaggatgaycagccacac

SEQ ID NO: 214 - DNA, 20 bp, synthetic construct
  misc_feature 1..20: note = PRIMER/PROBE
  source 1..20: mol_type = other DNA
cgcccattgtscaatattcc

SEQ ID NO: 215 - DNA, 24 bp, synthetic construct
  misc_feature 1..24: note = PRIMER/PROBE
  source 1..24: mol_type = other DNA
tgagacacggtccagactcctacg

SEQ ID NO: 216 - DNA, 24 bp, synthetic construct
  misc_feature 1..24: note = PRIMER/PROBE
  source 1..24: mol_type = other DNA
agcacatacagaatatgtccctgc

SEQ ID NO: 217 - DNA, 25 bp, synthetic construct
  misc_feature 1..25: note = PRIMER/PROBE
  source 1..25: mol_type = other DNA
acctgttaaccaacctacttgaggg

SEQ ID NO: 218 - DNA, 24 bp, synthetic construct
  misc_feature 1..24: note = PRIMER/PROBE
  source 1..24: mol_type = other DNA
ttgcaagacggactggcttagacc

SEQ ID NO: 219 - DNA, 20 bp, synthetic construct
  misc_feature 1..20: note = PRIMER/PROBE
  source 1..20: mol_type = other DNA
cctgatcggattggagaacc

SEQ ID NO: 220 - DNA, 23 bp, synthetic construct
  misc_feature 1..23: note = PRIMER/PROBE
  source 1..23: mol_type = other DNA
ctacctcttgaataggcgtaacc

SEQ ID NO: 221 - DNA, 24 bp, synthetic construct
  misc_feature 1..24: note = PRIMER/PROBE
  source 1..24: mol_type = other DNA
acgtcgcgcaagttcctgatagac

SEQ ID NO: 222 - DNA, 25 bp, synthetic construct
  misc_feature 1..25: note = PRIMER/PROBE
  source 1..25: mol_type = other DNA
tagtgactgctaatccaaatcacag

SEQ ID NO: 223 - DNA, 25 bp, synthetic construct
  misc_feature 1..25: note = PRIMER/PROBE
  source 1..25: mol_type = other DNA
gcacgagcaagatcattaccatagc

SEQ ID NO: 224 - DNA, 27 bp, synthetic construct
  misc_feature 1..27: note = PRIMER/PROBE
  source 1..27: mol_type = other DNA
agttatccaacaaggccaaactcaaca

SEQ ID NO: 225 - DNA, 20 bp, synthetic construct
  misc_feature 1..20: note = PRIMER/PROBE
  source 1..20: mol_type = other DNA
gagaggatgaycagccacac

SEQ ID NO: 226 - DNA, 20 bp, synthetic construct
  misc_feature 1..20: note = PRIMER/PROBE
  source 1..20: mol_type = other DNA
cgcccattgtscaatattcc

SEQ ID NO: 227 - DNA, 24 bp, synthetic construct
  misc_feature 1..24: note = PRIMER/PROBE
  source 1..24: mol_type = other DNA
tgagacacggtccagactcctacg

SEQ ID NO: 228 - DNA, 20 bp, synthetic construct
  misc_feature 1..20: note = PRIMER/PROBE
  source 1..20: mol_type = other DNA
aatcacagggcgtagttgtg

SEQ ID NO: 229 - DNA, 20 bp, synthetic construct
  misc_feature 1..20: note = PRIMER/PROBE
  source 1..20: mol_type = other DNA
acccaccagccaatcttagg

SEQ ID NO: 230 - DNA, 24 bp, synthetic construct
  misc_feature 1..24: note = PRIMER/PROBE
  source 1..24: mol_type = other DNA
tagcttgatcgccctcgatttggg

SEQ ID NO: 231 - DNA, 18 bp, synthetic construct
  misc_feature 1..18: note = PRIMER/PROBE
  source 1..18: mol_type = other DNA
gtgggatggaaagccacg

SEQ ID NO: 232 - DNA, 18 bp, synthetic construct
  misc_feature 1..18: note = PRIMER/PROBE
  source 1..18: mol_type = other DNA
cacttgcgggtctacagc

SEQ ID NO: 233 - DNA, 24 bp, synthetic construct
  misc_feature 1..24: note = PRIMER/PROBE
  source 1..24: mol_type = other DNA
ttactttgggcgaagccatgcaag

SEQ ID NO: 234 - DNA, 21 bp, synthetic construct
  misc_feature 1..21: note = PRIMER/PROBE
  source 1..21: mol_type = other DNA
cacctatggtaatgctcttgc

SEQ ID NO: 235 - DNA, 20 bp, synthetic construct
  misc_feature 1..20: note = PRIMER/PROBE
  source 1..20: mol_type = other DNA
ctggaactgctgacaatgcc

SEQ ID NO: 236 - DNA, 30 bp, synthetic construct; identical to SEQ ID NO: 136
  misc_feature 1..30: note = PRIMER/PROBE
  source 1..30: mol_type = other DNA

SEQ ID NO: 237 - DNA, 20 bp, synthetic construct
  misc_feature 1..20: note = PRIMER/PROBE
  source 1..20: mol_type = other DNA
gagaggatgaycagccacac

SEQ ID NO: 238 - DNA, 20 bp, synthetic construct
  misc_feature 1..20: note = PRIMER/PROBE
  source 1..20: mol_type = other DNA
cgcccattgtscaatattcc

SEQ ID NO: 239 - DNA, 24 bp, synthetic construct
  misc_feature 1..24: note = PRIMER/PROBE
  source 1..24: mol_type = other DNA
tgagacacggtccagactcctacg

SEQ ID NO: 240 - DNA, 18 bp, synthetic construct
  misc_feature 1..18: note = PRIMER/PROBE
  source 1..18: mol_type = other DNA
agatcagttgggtgcacg

SEQ ID NO: 241 - DNA, 20 bp, synthetic construct
  misc_feature 1..20: note = PRIMER/PROBE
  source 1..20: mol_type = other DNA
tgcttaatcagtgaggcacc

SEQ ID NO: 242 - DNA, 24 bp, synthetic construct
  misc_feature 1..24: note = PRIMER/PROBE
  source 1..24: mol_type = other DNA
atgaagccataccaaacgacgagc

SEQ ID NO: 243 - DNA, 20 bp, synthetic construct
  misc_feature 1..20: note = PRIMER/PROBE
  source 1..20: mol_type = other DNA
ctggagcgaaagatccacta

SEQ ID NO: 244 - DNA, 18 bp, synthetic construct
  misc_feature 1..18: note = PRIMER/PROBE
  source 1..18: mol_type = other DNA
atcgtccaccatccactg

SEQ ID NO: 245 - DNA, 22 bp, synthetic construct
  misc_feature 1..22: note = PRIMER/PROBE
  source 1..22: mol_type = other DNA
ccagatcggcgacaacgtcacc

SEQ ID NO: 246 - DNA, 20 bp, synthetic construct
  misc_feature 1..20: note = PRIMER/PROBE
  source 1..20: mol_type = other DNA
gagaggatgaycagccacac

SEQ ID NO: 247 - DNA, 20 bp, synthetic construct
  misc_feature 1..20: note = PRIMER/PROBE
  source 1..20: mol_type = other DNA
cgcccattgtscaatattcc

SEQ ID NO: 248 - DNA, 24 bp, synthetic construct
  misc_feature 1..24: note = PRIMER/PROBE
  source 1..24: mol_type = other DNA
tgagacacggtccagactcctacg

SEQ ID NO: 249 - DNA, 18 bp, synthetic construct
  misc_feature 1..18: note = PRIMER/PROBE
  source 1..18: mol_type = other DNA
ccgtgtatgttcagctat

SEQ ID NO: 250 - DNA, 18 bp, synthetic construct
  misc_feature 1..18: note = PRIMER/PROBE
  source 1..18: mol_type = other DNA
cttatccatcacgccttt

SEQ ID NO: 251 - DNA, 26 bp, synthetic construct
  misc_feature 1..26: note = PRIMER/PROBE
  source 1..26: mol_type = other DNA
tatgatgtcgataccgccaaatacca

SEQ ID NO: 252 - DNA, 18 bp, synthetic construct
  misc_feature 1..18: note = PRIMER/PROBE
  source 1..18: mol_type = other DNA
ctgtatgtcagcgatcat

SEQ ID NO: 253 - DNA, 19 bp, synthetic construct
  misc_feature 1..19: note = PRIMER/PROBE
  source 1..19: mol_type = other DNA
gatgccagtttgcttatcc

SEQ ID NO: 254 - DNA, 25 bp, synthetic construct
  misc_feature 1..25: note = PRIMER/PROBE
  source 1..25: mol_type = other DNA
aagtctgggtgagaacggtgtctat

SEQ ID NO: 255 - DNA, 19 bp, synthetic construct
  misc_feature 1..19: note = PRIMER/PROBE
  source 1..19: mol_type = other DNA
cagtcagtatgcgagtttc

SEQ ID NO: 256 - DNA, 18 bp, synthetic construct
  misc_feature 1..18: note = PRIMER/PROBE
  source 1..18: mol_type = other DNA
aaaattcgccaagccatc

SEQ ID NO: 257 - DNA, 25 bp, synthetic construct
  misc_feature 1..25: note = PRIMER/PROBE
  source 1..25: mol_type = other DNA
tgcataagccagtgcgtttttatat

SEQ ID NO: 258 - DNA, 20 bp, synthetic construct
  misc_feature 1..20: note = PRIMER/PROBE
  source 1..20: mol_type = other DNA
gagaggatgaycagccacac

SEQ ID NO: 259 - DNA, 20 bp, synthetic construct
  misc_feature 1..20: note = PRIMER/PROBE
  source 1..20: mol_type = other DNA
cgcccattgtscaatattcc

SEQ ID NO: 260 - DNA, 24 bp, synthetic construct
  misc_feature 1..24: note = PRIMER/PROBE
  source 1..24: mol_type = other DNA
tgagacacggtccagactcctacg

SEQ ID NO: 261 - DNA, 462 bp, synthetic construct
  misc_feature 1..462: note = CONTROL MIX
  source 1..462: mol_type = other DNA
tggccagaactgacaggcaaacagtggcagggtatccgcctgctgcacttagccacctatacggcaggcggcctaccgctgcagatccccgatgacgttagggataaagccgcattactgcatttttatcaaaactggcagccgcaatggactccgggcgctaagcgactttacgctaactccagcattggtctgtttggcgcgctggcggtgaaaccctcaggaatgagttacgaagaggcaatgaccagacgcgtcctgcaaccattaaaactggcgcatacctggattacggttccgcagaacgaacaaaaagattatgcctggggctatcgcgaagggaagcccgtacacgtttctccgggacaacttgacgccgaagcctatggcgtgaaatccagcgttattgatatggcccgctgggttcaggccaacatggatgccagccacgttcaggagaaa

SEQ ID NO: 262 - DNA, 121 bp, synthetic construct; identical to SEQ ID NO: 57
  misc_feature 1..121: note = CONTROL MIX
  source 1..121: mol_type = other DNA

SEQ ID NO: 263 - DNA, 321 bp, synthetic construct; identical to SEQ ID NO: 56
  misc_feature 1..321: note = CONTROL MIX
  source 1..321: mol_type = other DNA

SEQ ID NO: 264 - DNA, 84 bp, synthetic construct
  misc_feature 1..84: note = CONTROL MIX
  source 1..84: mol_type = other DNA
gagaggatgaccagccacactggaactgagacacggtccagactcctacgggaggcagcagtggggaatattgcacaatgggcg

SEQ ID NO: 265 - DNA, 553 bp, synthetic construct
  misc_feature 1..553: note = CONTROL MIX
  source 1..553: mol_type = other DNA
aatcacagggcgtagttgtgctctggaatgagaataagcagcaaggatttaccaataatcttaaacgggcgaaccaagcatttttacccgcatctacctttaaaattcccaatagcttgatcgccctcgatttgggcgtggttaaggatgaacaccaagtctttaagtgggatggacagacgcgcgatatcgccacttggaatcgcgatcataatctaatcaccgcgatgaaatattcagttgtgcctgtttatcaagaatttgcccgccaaattggcgaggcacgtatgagcaagatgctacatgctttcgattatggtaatgaggacatttcgggcaatgtagacagtttctggctcgacggtggtattcgaatttcggccacggagcaaatcagctttttaagaaagctgtatcacaataagttacacgtatcggagcgcagccagcgtattgtcaaacaagccatgctgaccgaagccaatggtgactatattattcgggctaaaactggatactcgactagaatcgaacctaagattggctggtgggt

SEQ ID NO: 266 - DNA, 175 bp, synthetic construct
  misc_feature 1..175: note = CONTROL MIX
  source 1..175: mol_type = other DNA
gcggagttagttattggctagttaaaaataaaattgaagttttttatcccggcccggggcacactcaagataacgtagtggtttggttacctgaaaagaaaattttattcggtggttgttttgttaaaccggacggtcttggtaatttgggtgacgcaaatttagaagcttggcc

SEQ ID NO: 267 - DNA, 297 bp, synthetic construct
  misc_feature 1..297: note = CONTROL MIX
  source 1..297: mol_type = other DNA
ggcggcgttgatgtccttcgggcggctggggtggcaacgtacgcatcaccgtcgacacgccggctagccgaggtagaggggaacgagattcccacgcactctctagaaggactctcatcgagcggggacgcagtgcgcttcggtccagtagaactcttctatcctggtgctgcgcattcgaccgacaacttagttgtgtacgtcccgtctgcgagtgtgctctatggtggttgtgcgattcatgagttgtcacgcacgtctgcggggaacgtggccgatgccgatctggctgaatgg

SEQ ID NO: 268 - DNA, 84 bp, synthetic construct; identical to SEQ ID NO: 264
  misc_feature 1..84: note = CONTROL MIX
  source 1..84: mol_type = other DNA

SEQ ID NO: 269 - DNA, 405 bp, synthetic construct
  misc_feature 1..405: note = CONTROL MIX
  source 1..405: mol_type = other DNA
aactttcacaggtgtgctgggtgcggtttctgtggcgaaaaaagagatggcgctgaatgatccggcggcaaaataccagccggagctggctctgccgcagtggaaggggatcacattgctggatctggctacctataccgcaggcggactgccgttacaggtgccggatgcggtaaaaagccgtgcggatctgctgaatttctatcagcagtggcagccgtcccggaaaccgggcgatatgcgtctgtatgcaaacagcagtatcggcctgtttggtgctctgaccgcaaacgcggcggggatgccgtatgagcagttgctgactgcacggatcctggcaccgctggggttatctcacacctttattactgtgccggaaagtgcgcaaagccagtatgcgtacgg

SEQ ID NO: 270 - DNA, 209 bp, synthetic construct; identical to SEQ ID NO: 62
  misc_feature 1..209: note = CONTROL MIX
  source 1..209: mol_type = other DNA

SEQ ID NO: 271 - DNA, 263 bp, synthetic construct
  misc_feature 1..263: note = CONTROL MIX
  source 1..263: mol_type = other DNA
gtttgatcgtcagggatggcggccgcgtgctggtggtcgataccgcctggaccgatgaccagaccgcccagatcctcaactggatcaagcaggagatcaacctgccggtcgcgctggcggtggtgactcacgcgcatcaggacaagatgggcggtatggacgcgctgcatgcggcggggattgcgacttatgccaatgcgttgtcgaaccagcttgccccgcaagaggggatggttgcggcgcaacacagcctgactttcgcc

SEQ ID NO: 272 - DNA, 84 bp, synthetic construct; identical to SEQ ID NO: 264
  misc_feature 1..84: note = CONTROL MIX
  source 1..84: mol_type = other DNA

SEQ ID NO: 273 - DNA, 520 bp, synthetic construct
  misc_feature 1..520: note = CONTROL MIX
  source 1..520: mol_type = other DNA
gctgctcaaggagcacaggatcccgggcatggcggtggccgtgctcaaggatggcaaggcccactatttcaattacggggtggccaaccgggagagcggggccagcgtcagcgagcagaccctgttcgagataggatccgtgagcaagaccctgactgcgaccctgggggcctatgcggtggtcaagggagcgatgcagctggatgacaaggcgagccggcacgcgccctggctcaagggatccgtctttgacagcatcaccatgggggagcttgccacctacagcgccggaggcctgccactgcaattccccgaggaggtggattcatccgagaagatgcgcgcctactaccgccagtgggcccctgtctattcgccgggctcccatcgccagtactccaaccccagcatagggctgttcggccacctggcggcgagcagcctgaagcagccatttgcccagttgatggagcagaccctgctgcccgggctcggcatgcaccacacctatgtcaatgtg

SEQ ID NO: 274 - DNA, 346 bp, synthetic construct
  misc_feature 1..346: note = CONTROL MIX
  source 1..346: mol_type = other DNA
aacagcctcagcagccggttacggaaaatacgttatttgaagtgggttcgctgagtaaaacgtttgctgccaccttggcgtcctatgcgcaggtgagcggtaagctgtctttggatcaaagcgttagccattacgttccagagttgcgtggcagcagctttgaccacgttagcgtactcaatgtgggcacgcatacctcaggcctacagctatttatgccggaagatattaaaaataccacacagctgatggcttatctaaaagcatggaaacctgccgatgcggctggaacccatcgcgtttattccaatatcggtactggtttgctagggatgattgcggcgaa

SEQ ID NO: 275 - DNA, 247 bp, synthetic construct
  misc_feature 1..247: note = CONTROL MIX
  source 1..247: mol_type = other DNA
gccgaggcttacgggatcaagaccggctcggcggatctgctgaagtttaccgaggccaacatggggtatcagggagatgccgcgctaaaaacgcggatcgcgctgacccataccggtttctactcggtgggagacatgactcaggggctgggttgggagagctacgcctatccgttgaccgagcaggcgctgctggcgggcaactccccggcggtgagcttccaggccaatccggttacgcgctttg

SEQ ID NO: 276 - DNA, 84 bp, synthetic construct; identical to SEQ ID NO: 264
  misc_feature 1..84: note = CONTROL MIX
  source 1..84: mol_type = other DNA

SEQ ID NO: 277 - DNA, 405 bp, synthetic construct; identical to SEQ ID NO: 269
  misc_feature 1..405: note = CONTROL MIX
  source 1..405: mol_type = other DNA

SEQ ID NO: 278 - DNA, 302 bp, synthetic construct
  misc_feature 1..302: note = CONTROL MIX
  source 1..302: mol_type = other DNA
tcggtaaagccgatgttgcggcgaacaaacccgtcaccccgcaaaccctgtttgagctgggctctataagtaaaaccttcaccggcgtactgggcggcgatgccattgcccggggtgaaatagcgctgggcgatccggtagcaaaatactggcctgagctcacgggcaagcagtggcagggcattcgcatgctggatctggcaacctataccgcaggcggtctgccgttacaggtgccggatgaggtcacggataccgcctctctgctgcgcttttatcaaaactggcagccgcagtggaag

SEQ ID NO: 279 - DNA, 106 bp, synthetic construct
  misc_feature 1..106: note = CONTROL MIX
  source 1..106: mol_type = other DNA
ccgaagcctatggcgtgaaatccagcgttattgatatggcccgctgggttcaggccaacatggatgccagccacgttcaggagaaaacgctccagcagggcattgc

SEQ ID NO: 280 - DNA, 84 bp, synthetic construct; identical to SEQ ID NO: 264
  misc_feature 1..84: note = CONTROL MIX
  source 1..84: mol_type = other DNA

SEQ ID NO: 281 - DNA, 487 bp, synthetic construct
  misc_feature 1..487: note = CONTROL MIX
  source 1..487: mol_type = other DNA
agcacatacagaatatgtccctgcatcaacatttaagatgctaaatgccttaattggactagaaaatcataaagctacaacaactgagattttcaaatgggacggtaaaaagagatcttatcccatgtgggaaaaagatatgactttaggtgatgccatggcactttcagcagttcctgtatatcaagaacttgcaagacggactggcttagacctaatgcaaaaagaagttaaacgggttggttttggtaatatgaacattggaacacaagttgataacttctggttggttggccccctcaagattacaccaatacaagaggttaattttgccgatgattttgcaaataatcgattaccctttaaattagagactcaagaagaagttaaaaaaatgcttctgattaaagaattcaatggtagtaaaatttatgcaaaaagcggctggggaatggatgtaacccctcaagtaggttggttaacaggt

SEQ ID NO: 282 - DNA, 278 bp, synthetic construct
  misc_feature 1..278: note = CONTROL MIX
  source 1..278: mol_type = other DNA
cctgatcggattggagaaccagaaaacggatattaatgaaatatttaaatggaagggcgagaaaaggtcatttaccgcttgggaaaaagacatgacactaggagaagccatgaagctttctgcagtcccagtctatcaggaacttgcgcgacgtatcggtcttgatctcatgcaaaaagaagtaaaacgtattggtttcggtaatgctgaaattggacagcaggttgataatttctggttggtaggaccattaaaggttacgcctattcaagaggtag

SEQ ID NO: 283 - DNA, 151 bp, synthetic construct
  misc_feature 1..151: note = CONTROL MIX
  source 1..151: mol_type = other DNA
tagtgactgctaatccaaatcacagcgcttcaaaatctgatgaaaaagcagagaaaattaaaaatttatttaacgaagtacacactacgggtgttttagttatccaacaaggccaaactcaacaaagctatggtaatgatcttgctcgtgc

SEQ ID NO: 284 - DNA, 84 bp, synthetic construct; identical to SEQ ID NO: 264
  misc_feature 1..84: note = CONTROL MIX
  source 1..84: mol_type = other DNA

SEQ ID NO: 285 - DNA, 553 bp, synthetic construct; identical to SEQ ID NO: 265
  misc_feature 1..553: note = CONTROL MIX
  source 1..553: mol_type = other DNA

SEQ ID NO: 286 - DNA, 376 bp, synthetic construct
  misc_feature 1..376: note = CONTROL MIX
  source 1..376: mol_type = other DNA
gtgggatggaaagccacgtttttttaaagcatgggacaaagattttactttgggcgaagccatgcaagcatctacagtgcctgtatatcaagaattggcacgtcgtattggtccaagcttaatgcaaagtgaattgcaacgtattggttatggcaatatgcaaataggcacggaagttgatcaattttggttgaaagggcctttgacaattacacctatacaagaagtaaagtttgtgtatgatttagcccaagggcaattgccttttaaacctgaagttcagcaacaagtgaaagagatgttgtatgtagagcgcagaggggagaatcgtctatatgctaaaagtggctggggaatggctgtagacccgcaagtg

SEQ ID NO: 287 - DNA, 376 bp, synthetic construct
  misc_feature 1..376: note = CONTROL MIX
  source 1..376: mol_type = other DNA
cacttgcgggtctacagccattccccagccacttttagcatatagacgattctcccctctgcgctctacatacaacatctctttcacttgttgctgaacttcaggtttaaaaggcaattgcccttgggctaaatcatacacaaactttacttcttgtataggtgtaattgtcaaaggccctttcaaccaaaattgatcaacttccgtgcctatttgcatattgccataaccaatacgttgcaattcactttgcattaagcttggaccaatacgacgtgccaattcttgatatacaggcactgtagatgcttgcatggcttcgcccaaagtaaaatctttgtcccatgctttaaaaaaacgtggctttccatcccac

SEQ ID NO: 288 - DNA, 203 bp, synthetic construct
  misc_feature 1..203: note = CONTROL MIX
  source 1..203: mol_type = other DNA
cacctatggtaatgctcttgcacgagcaaataaagaatatgtccctgcatcaacatttaagatgctaaatgctttaatcgggctagaaaatcataaagcaacaacaaatgagattttcaaatgggatggtaaaaaaagaacttatcctatgtgggagaaagatatgactttaggtgaggcaatggcattgtcagcagttccag

SEQ ID NO: 289 - DNA, 84 bp, synthetic construct; identical to SEQ ID NO: 264
  misc_feature 1..84: note = CONTROL MIX
  source 1..84: mol_type = other DNA

SEQ ID NO: 290 - DNA, 1625 bp, synthetic construct
  misc_feature 1..1625: note = CONTROL MIX
  source 1..1625: mol_type = other DNA
atgatgcagcatacttctgtgtggtaccgacgctcggtcagtccgtttgttcttgtgggagtgttgccgttttcttgaccgcgaccgccaatcttaccttttttgataaaatcagccaaacctatcccatcgcggacaatctcggctttgtgctgacgatcgctgtcgtgctctttggcgcgatgctactgatcaccacgctgttatcatcgtatcgctatgtgctaaagcctgtgttgattttgctattaatcatgggcgcggtgaccagttattttactgacacttatggcacggtctatgatacgaccatgctccaaaatgccctacagaccgaccaagccgagaccaaggatctattaaacgcagcgtttatcatgcgtatcattggtttgggtgtgctaccaagtttgcttgtggcttttgttaaggtggattatccgacttggggcaagggtttgatgcgccgattgggcttgatcgtggcaagtcttgcgctgattttactgcctgtggtggcgttcagcagtcattatgccagtttctttcgcgtgcataagccgctgcgtagctatgtcaatccgatcatgccaatctactcggtgggtaagcttgccagtattgagtataaaaaagccagtgcgccaaaagataccatttatcacgccaaagacgcggtacaagcaaccaagcctgatatgcgtaagccacgcctagtggtgttcgtcgtcggtgagacggcacgcgccgatcatgtcagcttcaatggctatgagcgcgatactttcccacagcttgccaagatcgatggcgtgaccaattttagcaatgtcacatcgtgcggcacatcgacggcgtattctgtgccgtgtatgttcagctatctgggcgcggatgagtatgatgtcgataccgccaaataccaagaaaatgtgctggatacgctggatcgcttgggcgtaagtatcttgtggcgtgataataattcggactcaaaaggcgtgatggataagctgccaaaagcgcaatttgccgattataaatccgcgaccaacaacgccatctgcaacaccaatccttataacgaatgccgcgatgtcggtatgctcgttggcttagatgactttgtcgctgccaataacggcaaagatatgctgatcatgctgcaccaaatgggcaatcacgggcctgcgtattttaagcgatatgatgaaaagtttgccaaattcacgccagtgtgtgaaggtaatgagcttgccaagtgcgaacatcagtccttgatcaatgcttatgacaatgccttgcttgccaccgatgatttcatcgctcaaagtatccagtggctgcagacgcacagcaatgcctatgatgtctcaatgctgtatgtcagcgatcatggcgaaagtctgggtgagaacggtgtctatctacatggtatgccaaatgcctttgcaccaaaagaacagcgcagtgtgcctgcatttttctggacggataagcaaactggcatcacgccaatggcaaccgataccgtcctgacccatgacgcgatcacgccgacattattaaagctgtttgatgtcaccgcggacaaagtcaaagaccgcaccgcattcatccgctga

SEQ ID NO: 291 - DNA, 1617 bp, synthetic construct
  misc_feature 1..1617: note = CONTROL MIX
  source 1..1617: mol_type = other DNA
atgacatcacatcactcttggtatcgctattctatcaatccttttgtgctgatgggtttggtggcgttatttttggcagcgacagcgaacctgacattttttgaaaaagcgatggcggtctatcctgtatcggataacttaggctttatcatctcaatggcggtggcggtgatgggtgctatgctactgattgtcgtgctgttatcctatcgctatgtgctaaagcctgtcctgattttgctactgattatgggtgcggtgacgagctattttaccgatacttatggcacggtctatgacaccaccatgctccaaaatgccatgcaaaccgaccaagccgagtctaaggacttgatgaatttggcgttttttgtgcgaattatcgggcttggcgtgttgccaagtgtgttggtcgcagttgccaaagtcaattatccaacatggggcaaaggtctgattcagcgtgcgatgacatggggtgtcagccttgtgctgttgcttgtgccgattggactatttagcagtcagtatgcgagtttctttcgggtgcataagccagtgcgtttttatatcaacccgattacgccgatttattcggtgggtaagcttgccagtatcgagtacaaaaaagccactgcgccaacagacaccatctatcatgccaaagacgccgtgcagaccaccaagccgagcgagcgtaagccacgcctagtggtgttcgtcgtcggtgagacggcgcgtgctgaccatgtgcagttcaatggctatggccgtgagactttcccgcagcttgccaaagttgatggcttggcgaattttagccaagtgacatcgtgtggcacatcgacggcgtattctgtgccgtgtatgttcagctatttgggtcaagatgactatgatgtcgataccgccaaataccaagaaaatgtgctagatacgcttgaccgcttgggtgtgggtatcttgtggcgtgataataattcagactcaaaaggcgtgatggataagctacctgccacgcagtattttgattataaatcagcaaccaacaataccatctgtaacaccaatccctataacgaatgccgtgatgtcggtatgcttgtcgggctagatgactatgtcagcgccaataatggcaaagatatgctcatcatgctacaccaaatgggcaatcatgggccggcgtactttaagcgttatgatgagcaatttgccaaattcacccccgtgtgcgaaggcaacgagcttgccaaatgcgaacaccaatcactcatcaatgcctatgacaatgcgctacttgcgactgatgattttatcgccaaaagcatcgattggctaaaaacgcatgaagcgaactacgatgtcgccatgctctatgtcagtgaccacggcgagagcttgggcgaaaatggtgtctatctgcatggtatgccaaatgcctttgcaccaaaagaacagcgagctgtgcctgcgtttttttggtcaaataatacgacattcaagccaactgccagcgatactgtgctgacgcatgatgcgattacgccaacactgcttaagctgtttgatgtcacagcgggcaaggtcaaagaccgcgcggcatttatccagtaa

SEQ ID NO: 292 - DNA, 84 bp, synthetic construct; identical to SEQ ID NO: 264
  misc_feature 1..84: note = CONTROL MIX
  source 1..84: mol_type = other DNA

SEQ ID NO: 293 - DNA, 750 bp, synthetic construct
  misc_feature 1..750: note = CONTROL MIX
  source 1..750: mol_type = other DNA
agatcagttgggtgcacgagtgggttacatcgaactggatctcaacagcggtaagatccttgagagttttcgccccgaagaacgttttccaatgatgagcacttttaaagttctgctatgtggtgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgacgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccagtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagca

SEQ ID NO: 294 - DNA, 372 bp, synthetic construct
  misc_feature 1..372: note = CONTROL MIX
  source 1..372: mol_type = other DNA
ctggagcgaaagatccactatcgccagcaggatctggtggactactcgccggtcagcgaaaaacaccttgccgacggcatgacggtcggcgaactctgcgccgccgccattaccatgagcgataacagcgccgccaatctgctgctggccaccgtcggcggccccgcaggattgactgcctttttgcgccagatcggcgacaacgtcacccgccttgaccgctgggaaacggaactgaatgaggcgcttcccggcgacgcccgcgacaccactaccccggccagcatggccgcgaccctgcgcaagctgctgaccagccagcgtctgagcgcccgttcgcaacggcagctgctgcagtggatggtggacgat

SEQ ID NO: 295 - DNA, 84 bp, synthetic construct; identical to SEQ ID NO: 264
  misc_feature 1..84: note = CONTROL MIX
  source 1..84: mol_type = other DNA

SEQ ID NO: 296 - DNA, 24 bp, synthetic construct
  misc_feature 1..24: note = Synthetic
  misc_feature 1: note = 6-FAM
  misc_feature 9: note = ZEN
  misc_feature 24: note = IABkFQ
  source 1..24: mol_type = other DNA
aggatggcaaggcccactatttca

SEQ ID NO: 297 - DNA, 24 bp, synthetic construct
  misc_feature 1..24: note = Synthetic
  misc_feature 1: note = HEX
  misc_feature 9: note = ZEN
  misc_feature 24: note = IABkFQ
  source 1..24: mol_type = other DNA
agccattacgttccagagttgcgt

SEQ ID NO: 298 - DNA, 24 bp, synthetic construct
  misc_feature 1..24: note = Synthetic
  misc_feature 1: note = TEX615
  misc_feature 24: note = 3IAbRQSp
  source 1..24: mol_type = other DNA
tctgctgaagtttrycgaggcmaa

SEQ ID NO: 299 - DNA, 24 bp, synthetic construct
  misc_feature 1..24: note = Synthetic
  misc_feature 1: note = TYE665
  misc_feature 24: note = IAbRQSp
  source 1..24: mol_type = other DNA
tgagacacggtccagactcctacg

SEQ ID NO: 300 - DNA, 24 bp, synthetic construct
  misc_feature 1..24: note = Synthetic
  misc_feature 1: note = 6-FAM
  misc_feature 9: note = ZEN
  misc_feature 24: note = IABkFQ
  source 1..24: mol_type = other DNA
aaaccgggcgatatgcgtctgtat

SEQ ID NO: 301 - DNA, 23 bp, synthetic construct
  misc_feature 1..23: note = Synthetic
  misc_feature 1: note = HEX
  misc_feature 9: note = ZEN
  misc_feature 23: note = IABkFQ
  source 1..23: mol_type = other DNA
gatgccattgcycgsggtgaaat

SEQ ID NO: 302 - DNA, 20 bp, synthetic construct
  misc_feature 1..20: note = Synthetic
  misc_feature 1: note = TEX615
  misc_feature 20: note = IAbRQSp
  source 1..20: mol_type = other DNA
atgttggcctgaacccagcg

(19) United States
(12) Patent Application Publication
(10) Pub. No.: US 2024/0150850 A1
(43) Pub. Date: May 9, 2024

(54) ASSAYS AND METHODS FOR DETERMINING MICROBIAL RESISTANCE

(71) Applicants: STRECK LLC, La Vista, NE (US); CREIGHTON UNIVERSITY, Omaha, NE (US)

(72) Inventors: Maria Torres-Gonzalez, Omaha, NE (US); Nancy Hanson, Omaha, NE (US); Joel Lechner, Omaha, NE (US); Stephanie Cossette, Omaha, NE (US); Cathy Scheer, Omaha, NE (US); Matthew R. Kreifels, Elkhorn, NE (US); Stacey Morrow, Omaha, NE (US); Christopher Connelly, Gretna, NE (US); Laura R. Porter, Omaha, NE (US); Randy Fowler, Broomfield, CO (US)

(21) Appl. No.: 18/332,590

(22) Filed: Jun. 9, 2023

Related U.S. Application Data
(62) Division of application No. 16/310,074, filed on Dec. 14, 2018, now Pat. No. 11,708,614.
(60) Provisional application No. 62/350,457, filed on Jun. 15, 2016.

(51) Int. Cl. C12Q 1/689; C12Q 1/6809; C12Q 1/6837; C12Q 1/6851; C12Q 1/686

(52) U.S. Cl. CPC C12Q 1/689; C12Q 1/6809; C12Q 1/6837; C12Q 1/6851; C12Q 1/686; C12Q 2545/101; C12Q 2600/106; C12Q 2600/16; C12Q 2600/166

(57) ABSTRACT

Assays and methods for detecting resistance to beta-lactam antibiotics including detection of multiple β-lactamase family specific gene targets by polymerase chain reaction or microarray. One or more kits including primers and/or probes for identification of β-lactamase genes selected from the group consisting of one or more of the following: MOX-like, FOX-like, ACC-like, ACT/MIR-like, CMY-2-like, DHA-like, CTX-M-14-like, CTX-M-15-like, VIM-like, NDM-like, IMP-like, KPC-like, and OXA-48-like, OXA-51-like, OXA-143-like, OXA-58-like, OXA-23-like, OXA-24/40-like, TEM-like, and SHV-like. A kit may also include one or more primers and/or probes for the identification a non-beta lactamase gene family which confers antibiotic resistance, such as the MCR-1 gene.

### CROSS-REFERENCE TO RELATED APPLICATIONS

This application is a Divisional of U.S. application Ser. No. 16/310,074, filed Dec. 14, 2018, which is a U.S. National Phase of PCT/US17/37700, filed Jun. 15, 2017, which claims the priority benefit under 35 U.S.C. § 119(e) of U.S. Provisional Patent Application No. 62/350,457, filed Jun. 15, 2016, which are incorporated herein by reference in their entirety.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “50035A_SubSeqListing.xml”, which was created on Jun. 8, 2023 and is 403,656 bytes in size. The subject matter of the Sequence Listing is incorporated herein in its entirety by reference.

### FIELD

The present teachings relate to assays and methods for detecting resistance to antibiotics. The present teachings provide for the detection of family specific gene targets including AmpC β-lactamases, metallo-β-lactamases, carbapenemases, and extended-spectrum β-Lactamases by multiplex real-time polymerase chain reaction.

### BACKGROUND

Bacterial resistance to antibiotics is a major public health issue. This resistance not only presents severe limitations to the ability to control and treat infection, but it also is difficult to identify and characterize in the laboratory. The significant increase in the resistance of pathogenic bacteria over the last 20 years, leads to extended periods of hospitalization, high morbidity and high mortality rates.

Enzymatic inactivation is the most common cause of resistance in terms of number of species and of antibiotics involved. As an example, β-lactamases are enzymes expressed by some bacteria. Such enzymes are capable of hydrolyzing the C—N bond of the β-lactam ring structure of a β-lactam antibiotic, effectively inactivating the antibiotic. Despite the existence of several β-lactamase inhibitors, the constant exposure of strains to antibiotics results in constant evolution of β-lactamases.

As a result, it becomes essential to be able to identify such resistant microorganisms and their resistance mechanisms as quickly as possible. Typically, biological samples can be tested for antibiotic resistance, but many test protocols are time consuming and/or limited in the types of resistance they are able to identify. It would therefore be beneficial to provide a test protocol for the simplified identification of resistance for all major β-lactamases.

One approach to the identification of β-lactamases has been to employ oligonucleotide primers specific for nucleic acid characteristic of certain β-lactamases with polymerase chain reaction to identify nucleic acid characteristics of family specific (β-lactamase enzymes in samples. See for example, U.S. Pat. Nos. 6,893,846 and 7,476,520, incorporated by reference herein. Another approach has been to employ oligonucleotide primers specific for nucleic acid characteristic of certain AmpC β-lactamases with multiplex polymerase chain reaction to detect the presence or absence of an AmpC β-lactamase gene and to identify nucleic acid characteristic of AmpC β-lactamase genes in samples. Multiplex polymerase chain reaction refers to the use of polymerase chain reaction to amplify several different DNA sequences simultaneously in single or multiple reactions. See for example, U.S. Pat. Nos. 7,045,291 and 7,521,547 incorporated by reference herein.

However, such primers have been limited with regards to the number of β-lactamase gene families or the number of gene targets that may be identified. Furthermore, such primers have been employed mainly with conventional polymerase chain reaction, which typically requires agarose gels to detect and analyze the PCR product(s). The use of agarose gel detection methods based on size discrimination may lead to poor resolution and difficulty in interpreting the data. Conventional polymerase chain reaction also lacks the sensitivity to detect endpoint variability from sample to sample and may not be automated. Real-time polymerase chain reaction allows for monitoring of reaction products as they are formed.

Detection of β-lactamases using real-time polymerase chain reaction and a single primer set may be limited to detection of a single β-lactamase gene family. See for example, United States Patent Application 2007/0248954 incorporated by reference herein. Multiplex real-time polymerase chain reaction has been designed for the identification of many AmpC β-lactamases simultaneously. See Geyer C N, Reisbig M D, Hanson N D. Development of a TaqMan® Multiplex PCR Assay for Detection of Plasmid-Mediated AmpC β-lactamase Genes. Journal of clinical microbiology. 2012 Aug 15:JCM-02038. The primer/probe combinations in this study, however, have been directed only to AmpC β-lactamases and are limited in the number of gene targets that may be identified.

Multiple factors such as primer and probe design, reaction conditions, and enzyme selection must all be considered when designing a working polymerase chain reaction. This complexity is compounded in multiplex PCR, in which multiple targets are detected simultaneously in the same tube. Balancing the concentrations of primers, probes, and control vectors provided as composite “multiplex PCR” mixes for an assay is a challenging aspect. It is extremely difficult to balance these ratios, as a change of concentration for any of these reagents, corresponding to just one of the genetic targets, may adversely affect detection of any other multiplex target in the reaction mix. If these concentrations are not balanced, one could expect a reduction in efficiency, sensitivity, and specificity. This would reduce confidence in the effectiveness of the assay to correctly identify the gene families identified with the described kits.

Therefore, there is a significant amount of time and technical know-how required to develop these assays into a reliable method. For example, the PCR master mixture, with DNA polymerase, is a customized formulation that permits the final assay to work. Concentrations of DNA polymerase and magnesium may have to be adjusted. The specific concentrations and ranges surrounding DNA polymerase and magnesium are required for the assay to work successfully. In addition to determining concentrations for all reagents, a PCR cycling protocol must be identified that is compatible with all reaction conditions and facilitates real-time multiplex polymerase chain reaction.

Accurate and rapid detection of antibiotic resistance is essential for surveillance, epidemiologic tracking, patient therapy, and infection control. Thus, a multiplex PCR based diagnostic assay should provide comprehensive genotypic characterization of β-lactamases and be versatile as well as providing rapid results. The present teachings make it possible to test a sample for the presence of antibiotic resistant microorganisms by identifying any of the major β-lactamases in one test. The present teachings provide for the detection of multiple family-specific β-lactamase gene targets, including but not limited to metallo-β-lactamases, carbapenemases, extended-spectrum β-lactamases, ampC chromosomal and/or plasmid-mediated AmpC β-lactamases, by multiplex real-time polymerase chain reaction.

The present teachings provide for a kit or kits including one or more primers and/or probes for identification of β-lactamase genes selected from the group consisting of one or more of the following: MOX-like, FOX-like, ACC-like, ACT/MIR--like, CMY-2-like, DHA-like, CTX-M-14-like, CTX-M-15-like, VIM-like, NDM-like, IMP-like, KPC-like, and OXA-48-like, OXA-51-like, OXA-143-like, OXA-58-like, OXA-23-like, OXA-24/40-like, TEM-like, and SHV-like. The kits or kits of the present teachings may provide control material for the aforementioned β-lactamase genes. The present teachings provide one or more of the following: primers, probes, controls, assay process and detection strategy for one or more of the following β-lactamases: extended-spectrum β-lactamases (ESBLs), metallo-β-lactamases (MBLs), carbapenem-resistant enterobacteriaceaes (CREs), and serine-dependent carbapenemases and plasmid-mediated ampC β-lactamases. A kit may also include one or more primers and/or probes for the identification of mobilized colistin-resistant (MCR) genes, a non-beta lactamase gene family that confers antibiotic resistance. The present teachings provide multiplex PCR assays which may test for any combination of these or are directed towards identification of a specific group. The present teachings provide assays with improved clinical sensitivity and analytical specificity of detection. The primer, probes, and control DNA sequences of the present teachings provide both an analytical and commercial advantage as they permit enhanced screening capabilities for detection of a larger number of genetic variants associated with genes conferring resistance to antibiotics in Gram-negative bacteria.

### SUMMARY

The present teachings provide a kit including one or more primers and/or probes for the identification by polymerase chain reaction, microarray, NGS-based target enrichment, and/or mass spectrometric characterization of one or more β-lactamase genes selected from the group consisting of: CMY, CTX-M, OXA, IMP, VIM, DHA, KPC, MOX, ACC, FOX, EBC, NDM, TEM, and SHV. The present teachings provide for one or more kits including primers and/or probes for identification of β-lactamase genes selected from the group consisting of one or more of the following: MOX-like, FOX-like, ACC-like, EBC-like, CMY-2-like, DHA-like, CTX-M-14-like, CTX-M-15-like, VIM-like, NDM-like, IMP-like, KPC-like, and OXA-48-like, OXA-51-like, OXA-143-like, OXA-58-like, OXA-23-like, OXA-24/40-like, TEM-like, and SHV-like. A kit may also include one or more primers and/or probes for the identification of a non-beta lactamase gene family which confers antibiotic resistance. A kit may include one or more primers and/or probes for the identification by polymerase chain reaction or microarray of MCR gene variants. Primers and probes may also be made compatible with next-generation sequencing and mass spectrometry.

### BRIEF DESCRIPTION OF DRAWINGS

FIG. 1 depicts an amplification plot of an exemplary mix 1 of a kit including ampC gene targets.

FIG. 2 depicts an amplification plot of an exemplary mix 2 of a kit including ampC gene targets.

FIG. 3 depicts an amplification plot of an exemplary mix 1 of a kit including β-lactamase gene targets.

FIG. 4 depicts an amplification plot of an exemplary mix 2 of a kit including β-lactamase gene targets.

FIG. 5 depicts an amplification plot of an exemplary mix 3 of a kit including β-lactamase gene targets.

FIG. 6 depicts an amplification plot of an exemplary internal control mix of a kit including MCR gene targets.

FIG. 7 depicts an amplification plot of an exemplary mix 1 of a kit including OXA gene targets.

FIG. 8 depicts an amplification plot of an exemplary mix 2 of a kit including OXA gene targets.

FIG. 9 depicts an amplification plot of an exemplary internal control mix of a kit including SHV-TEM gene targets.

### DETAILED DESCRIPTION

The explanations and illustrations presented herein are intended to acquaint others skilled in the art with the teachings, its principles, and its practical application. Those skilled in the art may adapt and apply the teachings in its numerous forms, as may be best suited to the requirements of a particular use. Accordingly, the specific embodiments of the present teachings as set forth are not intended as being exhaustive or limiting of the teachings. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

Bacterial resistance to antibiotics poses a global threat to public health and in recent years has shown an increase in mortality rates and the potential to spread through the population. Of these resistance mechanisms, β-lactamases are enzymes that cleave β-lactam rings rendering the β-lactam family of antibiotics ineffective for treatment of clinically-important Gram-negative bacterial infections. Specifically, β-lactamases confer resistance to penicillins, cephamycins, and, in some cases, carbapenems. β-lactam-resistant Gram-negative organisms, producing multiple or plasmid-mediated β-lactamases, are difficult to identify phenotypically and necessitate more specific detection methods to identify clinically important β-lactamases. Genetic identification of these resistance mechanisms is critical for active surveillance and infection control. Because these antibiotics are often selected for the management and prevention of infectious disease, the presence and characteristics of specific β-lactamases play a critical role in selecting the appropriate antibiotic therapy.

AmpC β-lactamases are clinically important cephalosporinases that are resistant to most β-lactam antibiotics. AmpC enzymes are chromosomally encoded in many bacterial species and can be inducible and overexpressed as a consequence of mutation. Overexpression can lead to resistance to most β-lactam antibiotics. The occurrence of transmissible plasmids with acquired genes for AmpC β-lactamases often result in increased β-lactamase production, compared to chromosomally-expressed ampC genes. Additionally, plasmid-mediated AmpC β-lactamases can appear in organisms lacking or having low-level expression of a chromosomal ampC gene. Resistance due to plasmid-mediated AmpC enzymes can be broad in spectrum and often hard to detect. As such, it is clinically useful to detect and discriminate between plasmid-mediated and chromosomally expressed AmpC β-lactamases.

The present teachings relate to assays and methods for detecting Gram-negative bacteria resistant to beta-lactam antibiotics from a biological sample. β-lactam antiobiotics are all antiobiotic agents that contain a β-lactam ring in their molecular structures. β-lactam antiobiotics include penicillins, cephalsoprins, carbapenems and monobactams. Antibiotic resistant organisms may produce one or more enzymes known as β-lactamases that provide resistance to β-lactam antibiotics. β-lactamases may confer resistance by the bacteria to antibiotics, which is plasmid-mediated and/or chromosomally expressed making detection difficult.

β-lactamases may be classified based on molecular structure. The four major classes include A to D. Class A, C and D β-lactamases are serine based. Class B β-lactamases, also known as metallo-beta-lactamases, are zinc based.

Extended spectrum β-lactamases (ESBLs) are enzymes that confer bacterial resistance to certain categories of antibiotics, such as third-generation cephalsoprins and monobactams. The presence of an ESBL-producing organism in a clinical infection can cause treatment failure if one of the above classes of drugs is used. Detection of ESBLs can be difficult because they have different levels of activity against various cephalosporins. Thus genetic identification of the exact enzyme can facilitate selection of the optimal antimicrobial agent, which is critical to determine the most effective treatment response.

First-generation cephalosporins include cefalexin, cefaloridine, cefalotin, cefazolin, cefadroxil, cefazedone, cefatrizine, cefapirin, cefradine, cefacetrile, cefrodaxine, ceftezole. Second-generation cephalosporins include cefoxitin, cefuroxime, cefamandole, cefaclor, cefotetan, cefonicide, cefotiam, loracarbef, cefmetazole, cefprozil, ceforanide. Third-generation cephalosporins include cefotaxime, ceftazidime, cefsulodine, ceftriaxone, cefmenoxime, latamoxef, ceftizoxime, cefixime, cefodizime, cefetamet, cefpiramide, cefoperazone, cefpodoxime, ceftibuten, cefdinir, cefditoren, ceftriaxone, cefoperazone, cefbuperazone. Fourth-generation cephalosporins include cefepime and cefpirome.

β-lactamase producing bacteria may include Gram-negative bacteria such as those found in the following genera: Pseudomonas, Escherichia, Salmonella, Shigella, Enterobacter, Klebsiella, Serratia, Proteus, Campylobacter, Haemophilus, Morganella, Vibrio, Yersinia, Acinetobacter, Branhamella, Neisseria, Burkholderia, Citrobacter, Hafnia, Edwardsiella, Aeromonas, Moraxella, Pasteurella, Providencia and Legionella.

Antibiotic resistance is intended to mean any type of mechanism which allows a microorganism to render a treatment partially or completely ineffective on the microorganism, guaranteeing its survival. β-lactam antibiotic resistance is intended to mean any type of β-lactamase-based mechanism which allows a microorganism to render a treatment partially or completely ineffective on the microorganism, guaranteeing its survival. For example, wherein the mechanism is related to the expression of an enzyme belonging to the β-lactamase group including extended-spectrum β-lactamase or of an enzyme belonging to the group of class C cephalosporinases.

Biological sample is intended to mean a clinical sample, derived from a specimen of biological fluid, or a food sample, derived from any type of food or drink, or from an agricultural source, such as animals, soil, water, or air, or from a surface such as with a biofilm. This sample may thus be liquid or solid. For example the biological sample may be a clinical sample of blood, plasma, urine or feces, or of rectal, nose, throat, skin, wound or cerebrospinal fluid specimens.

The present teachings relate to assays and methods for detecting resistance to beta-lactam antibiotics. The present teachings may detect β-lactamase gene targets which are chromosomally encoded and/or plasmid mediated. The present teachings provide for the detection of family specific gene targets relating to β-lactamase genes including AmpC β-lactamases. The β-lactamase genes detected with the present teachings may include those classified into molecular groups A through D. The β-lactamase genes detected with the present teachings may include those classified into functional groups 1 through 3.

The present teachings relate to assays and methods for detecting resistance of one or more gene beta lactamase gene families including like genes. A like gene may be a beta-lactamase that has one or more of the following: similar amino acid sequence, similar function and similar antibiotic susceptibility profiles. A like gene may be considered as like the target gene detected with the present teachings. For example, OXA-48-like enzymes may include: OXA-48, OXA-48b, OXA-162, OXA-163, OXA-181, OXA-199, OXA-204, OXA-232, OXA-244, OXA-245 and OXA-24.

The present teachings provide one or more primers and/or probes for the identification of one or more β-lactamase genes selected from the group consisting of: CMY, CTX-M, OXA, IMP, VIM, DHA, KPC, MOX, ACC, FOX, EBC, NDM, TEM, and SHV. The present teachings provide one or more primers and/or probes for the identification of β-lactamase genes selected from the group consisting of one or more of the following: MOX-like, FOX-like, ACC-like, EBC-like, CMY-2-like, DHA-like, CTX-M-14-like, CTX-M-15-like, VIM-like, NDM-like, IMP-like, KPC-like, and OXA-48-like, OXA-51-like, OXA-143-like, OXA-58-like, OXA-23-like, OXA-24/40-like, TEM-like, and SHV-like. The present teachings provide one or more primers and/or probes for the identification of a non-beta lactamase gene family which confers antibiotic resistance. For example, one or more primers and/or probes for the identification of MCR gene variants. The primers and/or probes of the present teachings may be included in one or more kits. The one or more kits may be used for identification with any of the following: polymerase chain reaction, microarray, NGS-based target enrichment, and/or mass spectrometric characterization.

Exemplary sequences for primers and probes for of the present teachings are depicted in Table 1. [SEQ. ID NOS 67-260] Primers and/or probes may be degenerate at any nucleotide position. Primers and/or probes may not be degenerate at any nucleotide position. Any suitable fluorophore and/or quencher and nucleic acid sequence combination may be used. For example, a probe may be labeled with a fluorescent tag at one end and a fluorescent quencher at the other end. For example, a probe may be labeled with a fluorescent tag at one end and a fluorescent quencher at the other end. For example, two fluorescent quenchers may be included at one end or within the probe sequence. It is contemplated that the probe sequences of the present teachings may be labeled with any suitable fluorophore and quencher combinations. For example, any fluorophore of the present teachings may be attached to any probe DNA sequence of the present teachings.

TABLE 1

| | Primer/Probe Sequence |
|---|---|
| SEQ ID NO. 67 | TGGCCAGAACTGACAGGCAAA |
| SEQ ID NO. 68 | TTTCTCCTGAACGTGGCTGGC |
| SEQ ID NO. 69 | 56-FAM/ACGCTAACT/ZEN/CCAGCATTGGTCTGT/3IABkFQ/ |
| SEQ ID NO. 70 | CCGTCACGCTGTTGTTAGG |
| SEQ ID NO. 71 | GCTGTGTTAATCAATGCCACAC |
| SEQ ID NO. 72 | 5HEX/AACTTGCCG/ZEN/AATTAGAGCRGCAGT/3IABkFQ |
| SEQ ID NO. 73 | CGTTTCGTCTGGATCGCAC |
| SEQ ID NO. 74 | GCTGGGTAAAATAGGTCACC |
| SEQ ID NO. 75 | 5TEX615/TATCATTGGTGGTGCCGTAGTCGC/3IAbRQSp |
| SEQ ID NO. 76 | GAGAGGATGAYCAGCCACAC |
| SEQ ID NO. 77 | CGCCCATTGTSCAATATTCC |
| SEQ ID NO. 78 | 5TYE665/TGAGACACGGTCCAGACTCCTACG/3IAbRQSp |
| SEQ ID NO. 79 | AATCACAGGGCGTAGTTGTG |
| SEQ ID NO. 80 | ACCCACCAGCCAATCTTAGG |
| SEQ ID NO. 81 | 56-FAM/TAGCTTGAT/ZEN/CGCCCTCGATTTGGG/3IABkFQ/ |
| SEQ ID NO. 82 | GCGGAGTTAACTATTGGCTAG |
| SEQ ID NO. 83 | GGCCAAGCTTCTATATTTGCG |
| SEQ ID NO. 84 | 5HEX/TTRTTYGGT/ZEN/GGTTGYTTTRTTAA/3IABkFQ |
| SEQ ID NO. 85 | GCGGAGTTARYTATTGGCTAG |
| SEQ ID NO. 86 | GGCCAAGCYTCTAWATTTGCG |
| SEQ ID NO. 87 | /5HEX/CCGGACGGT/ZEN/CTTGGTAATTTGGGT/3IABkFQ/ |
| SEQ ID NO. 88 | /5HEX/CCGTACGGT/ZEN/TTAGGCAATTTGGGT/3IABkFQ/ |
| SEQ ID NO. 89 | GGCGGCGTTGATGTCCTTCG |
| SEQ ID NO. 90 | CCATTCAGCCAGATCGGCATC |
| SEQ ID NO. 91 | 5TEX615/AGCTCTTCTATCCTGGTGCTGCG/3IAbRQSp |
| SEQ ID NO. 92 | AACTTTCACAGGTGTGCTGGGT |
| SEQ ID NO. 93 | CCGTACGCATACTGGCTTTGC |
| SEQ ID NO. 94 | 56-FAM/AAACCGGGC/ZEN/GATATGCGTCTGTAT/3IABkFQ/ |
| SEQ ID NO. 95 | GTATCGCCGTCTAGTTCTGC |
| SEQ ID NO. 96 | CCTTGAATGAGCTGCACAGTGG |
| SEQ ID NO. 97 | 5HEX/TCGTCGCGG/ZEN/AACCATTCGCTAAA/3IABkFQ/ |
| SEQ ID NO. 98 | GTTTGATCGTCAGGGATGGC |
| SEQ ID NO. 99 | GGCGAAAGTCAGGCTGTG |
| SEQ ID NO. 100 | 5TEX615/CATCAGGACAAGATGGGCGGTATG/3IAbRQSp |
| SEQ ID NO. 101 | GCTGCTCAAGGAGCACAGGAT |
| SEQ ID NO. 102 | CACATTGACATAGGTGTGGTGC |
| SEQ ID NO. 103 | 56-FAM/AGGATGGCA/ZEN/AGGCCCACTATTTCA/3IABkFQ |
| SEQ ID NO. 104 | AACAGCCTCAGCAGCCGGTTA |
| SEQ ID NO. 105 | TTCGCCGCAATCATCCCTAGC |
| SEQ ID NO. 106 | 5HEX/AGCCATTAC/ZEN/GTTCCAGAGTTGCGT/3IABkFQ |
| SEQ ID NO. 107 | GCCGAGGCTTACGGGATCAAG |
| SEQ ID NO. 108 | CAAAGCGCGTAACCGGATTGG |
| SEQ ID NO. 109 | 5TEX615/TCTGCTGAAGTTTRYCGAGGCMAA/3IAbRQSp |
| SEQ ID NO. 110 | AACTTTCACAGGTGTGCTGGGT |
| SEQ ID NO. 111 | CCGTACGCATACTGGCTTTGC |
| SEQ ID NO. 112 | 56-FAM/AAACCGGGC/ZEN/GATATGCGTCTGTAT/3IABkFQ/ |
| SEQ ID NO. 113 | CTGGGTTCTATAAGTAAAACCTTCACCGG |
| SEQ ID NO. 114 | CTTCCACTGCGGCTGCCAGTT |
| SEQ ID NO. 115 | 5HEX/GATGCCATT/ZEN/GCYCGSGGTGAAAT/3IABkFQ |
| SEQ ID NO. 116 | CCGAAGCCTATGGCGTGAAATCC |
| SEQ ID NO. 117 | GCAATGCCCTGCTGGAGCG |
| SEQ ID NO. 118 | 5TEX615/ATGTTGGCCTGAACCCAGCG/3IAbRQSp |
| SEQ ID NO. 119 | AGCACATACAGAATATGTCCCTGC |
| SEQ ID NO. 120 | ACCTGTTAACCAACCTACTTGAGGG |
| SEQ ID NO. 121 | /56-FAM/TTGCAAGACGGACTGGCTTAGACC/3BHQ_1/ |
| SEQ ID NO. 122 | CCTGATCGGATTGGAGAACC |
| SEQ ID NO. 123 | CTACCTCTTGAATAGGCGTAACC |
| SEQ ID NO. 124 | /5TEX615/ACGTCGCGCAAGTTCCTGATAGAC/3IAbRQSp/ |
| SEQ ID NO. 125 | TAGTGACTGCTAATCCAAATCACAG |
| SEQ ID NO. 126 | GCACGAGCAAGATCATTACCATAGC |
| SEQ ID NO. 127 | /5HEX/AGTTATCCAACAAGGCCAAACTCAACA/3BHQ_1/ |
| SEQ ID NO. 128 | AATCACAGGGCGTAGTTGTG |
| SEQ ID NO. 129 | ACCCACCAGCCAATCTTAGG |
| SEQ ID NO. 130 | /5HEX/TAGCTTGATCGCCCTCGATTTGGG/3BHQ_1/ |
| SEQ ID NO. 131 | GTGGGATGGAAAGCCACG |
| SEQ ID NO. 132 | CACTTGCGGGTCTACAGC |
| SEQ ID NO. 133 | /56-FAM/TTACTTTGGGCGAAGCCATGCAAG/3BHQ_1/ |
| SEQ ID NO. 134 | CACCTATGGTAATGCTCTTGC |
| SEQ ID NO. 135 | CTGGAACTGCTGACAATGCC |
| SEQ ID NO. 136 | /5TEX615/TGGGAGAAAGATATGACTTTAGGTGAGGCA/3IAbRQSp/ |
| SEQ ID NO. 137 | CCGTGTATGTTCAGCTAT |
| SEQ ID NO. 138 | CTTATCCATCACGCCTTT |
| SEQ ID NO. 139 | /5TEX615/TATGATGTCGATACCGCCAAATACCA/3IAbRQSp/ |
| SEQ ID NO. 140 | CTGTATGTCAGCGATCAT |
| SEQ ID NO. 141 | GATGCCAGTTTGCTTATCC |
| SEQ ID NO. 142 | /56FAM/AAGTCTGGG/ZEN/TGAGAACGGTGTCTAT/3IABkFQ |
| SEQ ID NO. 143 | CAGTCAGTATGCGAGTTTC |
| SEQ ID NO. 144 | AAAATTCGCCAAGCCATC, |
| SEQ ID NO. 145 | /5HEX/TGCATAAGC/ZEN/CAGTGCGTTTTTATAT/3IABkFQ |
| SEQ ID NO. 146 | AGATCAGTTGGGTGCACG |
| SEQ ID NO. 147 | TGCTTAATCAGTGAGGCACC |
| SEQ ID NO. 148 | /56-FAM/ATGAAGCCA/ZEN/TACCAAACGACGAGC/3IABkFQ/ |
| SEQ ID NO. 149 | CTGGAGCGAAAGATCCACTA |
| SEQ ID NO. 150 | ATCGTCCACCATCCACTG |
| SEQ ID NO. 151 | /5HEX/CCAGATCGG/ZEN/CGACAACGTCACC/3IABkFQ/ |
| SEQ ID NO. 152 | TGGCCAGAACTGACAGGCAAA |
| SEQ ID NO. 153 | TTTCTCCTGAACGTGGCTGGC |
| SEQ ID NO. 154 | 56-FAM/ACGCTAACT/ZEN/CCAGCATTGGTCTGT/3IABkFQ/ |
| SEQ ID NO. 155 | CCGTCACGCTGTTGTTAGG |
| SEQ ID NO. 156 | GCTGTGTTAATCAATGCCACAC |
| SEQ ID NO. 157 | 5HEX/AACTTGCCG/ZEN/AATTAGAGCRGCAGT/3IABkFQ |
| SEQ ID NO. 158 | CGTTTCGTCTGGATCGCAC |
| SEQ ID NO. 159 | GCTGGGTAAAATAGGTCACC |
| SEQ ID NO. 160 | 5TEX615/TATCATTGGTGGTGCCGTAGTCGC/3IAbRQSp |
| SEQ ID NO. 161 | GAGAGGATGAYCAGCCACAC |
| SEQ ID NO. 162 | CGCCCATTGTSCAATATTCC |
| SEQ ID NO. 163 | 5TYE665/TGAGACACGGTCCAGACTCCTACG/3IAbRQSp |
| SEQ ID NO. 164 | AATCACAGGGCGTAGTTGTG |
| SEQ ID NO. 165 | ACCCACCAGCCAATCTTAGG |
| SEQ ID NO. 166 | 56-FAM/TAGCTTGAT/ZEN/CGCCCTCGATTTGGG/13IABkFQ/ |
| SEQ ID NO. 167 | GCGGAGTTAACTATTGGCTAG |
| SEQ ID NO. 168 | GGCCAAGCTTCTATATTTGCG |
| SEQ ID NO. 169 | 5HEX/TTRTTYGGT/ZEN/GGTTGYTTTRTTAA/3IABkFQ |
| SEQ ID NO. 170 | GCGGAGTTARYTATTGGCTAG |
| SEQ ID NO. 171 | GGCCAAGCYTCTAWATTTGCG |
| SEQ ID NO. 172 | /5HEX/CCGGACGGT/ZEN/CTTGGTAATTTGGGT/3IABkFQ/ |
| SEQ ID NO. 173 | /5HEX/CCGTACGGT/ZEN/TTAGGCAATTTGGGT/3IABkFQ |
| SEQ ID NO. 174 | GGCGGCGTTGATGTCCTTCG |
| SEQ ID NO. 175 | CCATTCAGCCAGATCGGCATC |
| SEQ ID NO. 176 | 5TEX615/AGCTCTTCTATCCTGGTGCTGCG/3IAbRQSp |
| SEQ ID NO. 177 | GAGAGGATGAYCAGCCACAC |
| SEQ ID NO. 178 | CGCCCATTGTSCAATATTCC |
| SEQ ID NO. 179 | 5TYE665/TGAGACACGGTCCAGACTCCTACG/3IAbRQSp |
| SEQ ID NO. 180 | AACTTTCACAGGTGTGCTGGGT |
| SEQ ID NO. 181 | CCGTACGCATACTGGCTTTGC |
| SEQ ID NO. 182 | 56-FAM/AAACCGGGC/ZEN/GATATGCGTCTGTAT/3IABkFQ/ |
| SEQ ID NO. 183 | GTATCGCCGTCTAGTTCTGC |
| SEQ ID NO. 184 | CCTTGAATGAGCTGCACAGTGG |
| SEQ ID NO. 185 | 5HEX/TCGTCGCGG/ZEN/AACCATTCGCTAAA/3IABkFQ/ |
| SEQ ID NO. 186 | GTTTGATCGTCAGGGATGGC |
| SEQ ID NO. 187 | GGCGAAAGTCAGGCTGTG |
| SEQ ID NO. 188 | 5TEX615/CATCAGGACAAGATGGGCGGTATG/3IAbRQSp |
| SEQ ID NO. 189 | GAGAGGATGAYCAGCCACAC |
| SEQ ID NO. 190 | CGCCCATTGTSCAATATTCC |
| SEQ ID NO. 191 | 5TYE665/TGAGACACGGTCCAGACTCCTACG/3IAbRQSp |
| SEQ ID NO. 192 | GCTGCTCAAGGAGCACAGGAT |
| SEQ ID NO. 193 | CACATTGACATAGGTGTGGTGC |
| SEQ ID NO. 194 | 56-FAM/AGGATGGCA/ZEN/AGGCCCACTATTTCA/3IABkFQ |
| SEQ ID NO. 195 | AACAGCCTCAGCAGCCGGTTA |
| SEQ ID NO. 196 | TTCGCCGCAATCATCCCTAGC |
| SEQ ID NO. 197 | 5H EX/AGCCATTAC/ZEN/GTTCCAGAGTTGCGT/3IABkFQ |
| SEQ ID NO. 198 | GCCGAGGCTTACGGGATCAAG |
| SEQ ID NO. 199 | CAAAGCGCGTAACCGGATTGG |
| SEQ ID NO. 200 | 5TEX615/TCTGCTGAAGTTTRYCGAGGCMAA/3IAbRQSp |
| SEQ ID NO. 201 | GAGAGGATGAYCAGCCACAC |
| SEQ ID NO. 202 | CGCCCATTGTSCAATATTCC |
| SEQ ID NO. 203 | 5TYE665/TGAGACACGGTCCAGACTCCTACG/3IAbRQSp |
| SEQ ID NO. 204 | AACTTTCACAGGTGTGCTGGGT |
| SEQ ID NO. 205 | CCGTACGCATACTGGCTTTGC |
| SEQ ID NO. 206 | 56-FAM/AAACCGGGC/ZEN/GATATGCGTCTGTAT/3IABkFQ |
| SEQ ID NO. 207 | CTGGGTTCTATAAGTAAAACCTTCACCGG |
| SEQ ID NO. 208 | CTTCCACTGCGGCTGCCAGTT |
| SEQ ID NO. 209 | 5HEX/GATGCCATT/ZEN/GCYCGSGGTGAAAT/3IABkFQ |
| SEQ ID NO. 210 | CCGAAGCCTATGGCGTGAAATCC |
| SEQ ID NO. 211 | GCAATGCCCTGCTGGAGCG |
| SEQ ID NO. 212 | 5TEX615/ATGTTGGCCTGAACCCAGCG/3IAbRQSp |
| SEQ ID NO. 213 | GAGAGGATGAYCAGCCACAC |
| SEQ ID NO. 214 | CGCCCATTGTSCAATATTCC |
| SEQ ID NO. 215 | 5TYE665/TGAGACACGGTCCAGACTCCTACG/3IAbRQSp |
| SEQ ID NO. 216 | AGCACATACAGAATATGTCCCTGC |
| SEQ ID NO. 217 | ACCTGTTAACCAACCTACTTGAGGG |
| SEQ ID NO. 218 | /56-FAM/TTGCAAGACGGACTGGCTTAGACC/3BHQ_1/ |
| SEQ ID NO. 219 | CCTGATCGGATTGGAGAACC |
| SEQ ID NO. 220 | CTACCTCTTGAATAGGCGTAACC |
| SEQ ID NO. 221 | /5TEX615/ACGTCGCGCAAGTTCCTGATAGAC/3IAbRQSp/ |
| SEQ ID NO. 222 | TAGTGACTGCTAATCCAAATCACAG |
| SEQ ID NO. 223 | GCACGAGCAAGATCATTACCATAGC |
| SEQ ID NO. 224 | /5HEX/AGTTATCCAACAAGGCCAAACTCAACA/3BHQ_1/ |
| SEQ ID NO. 225 | GAGAGGATGAYCAGCCACAC |
| SEQ ID NO. 226 | CGCCCATTGTSCAATATTCC |
| SEQ ID NO. 227 | 5TYE665/TGAGACACGGTCCAGACTCCTACG/3IAbRQSp |
| SEQ ID NO. 228 | AATCACAGGGCGTAGTTGTG |
| SEQ ID NO. 229 | ACCCACCAGCCAATCTTAGG |
| SEQ ID NO. 230 | /5HEX/TAGCTTGATCGCCCTCGATTTGGG/3BHQ_1/ |
| SEQ ID NO. 231 | GTGGGATGGAAAGCCACG |
| SEQ ID NO. 232 | CACTTGCGGGTCTACAGC |
| SEQ ID NO. 233 | /56-FAM/TTACTTTGGGCGAAGCCATGCAAG/3BHQ_1/ |
| SEQ ID NO. 234 | CACCTATGGTAATGCTCTTGC, |
| SEQ ID NO. 235 | CTGGAACTGCTGACAATGCC |
| SEQ ID NO. 236 | /5TEX615/TGGGAGAAAGATATGACTTTAGGTGAGGCA/3IAbRQSp/ |
| SEQ ID NO. 237 | GAGAGGATGAYCAGCCACAC |
| SEQ ID NO. 238 | CGCCCATTGTSCAATATTCC |
| SEQ ID NO. 239 | 5TYE665/TGAGACACGGTCCAGACTCCTACG/3IAbRQSp |
| SEQ ID NO. 240 | AGATCAGTTGGGTGCACG |
| SEQ ID NO. 241 | TGCTTAATCAGTGAGGCACC |
| SEQ ID NO. 242 | /56-FAM/ATGAAGCCA/ZEN/TACCAAACGACGAGC/3IABkFQ/ |
| SEQ ID NO. 243 | CTGGAGCGAAAGATCCACTA |
| SEQ ID NO. 244 | ATCGTCCACCATCCACTG |
| SEQ ID NO. 245 | /5HEX/CCAGATCGG/ZEN/CGACAACGTCACC/3IABkFQ/ |
| SEQ ID NO. 246 | GAGAGGATGAYCAGCCACAC |
| SEQ ID NO. 247 | CGCCCATTGTSCAATATTCC |
| SEQ ID NO. 248 | 5TYE665/TGAGACACGGTCCAGACTCCTACG/3IAbRQSp |
| SEQ ID NO. 249 | CCGTGTATGTTCAGCTAT |
| SEQ ID NO. 250 | CTTATCCATCACGCCTTT |
| SEQ ID NO. 251 | /5TEX615/TATGATGTCGATACCGCCAAATACCA/3IAbRQSp/ |
| SEQ ID NO. 252 | CTGTATGTCAGCGATCAT |
| SEQ ID NO. 253 | GATGCCAGTTTGCTTATCC |
| SEQ ID NO. 254 | /56FAM/AAGTCTGGG/ZEN/TGAGAACGGTGTCTAT/3IABkFQ/ |
| SEQ ID NO. 255 | CAGTCAGTATGCGAGTTTC |
| SEQ ID NO. 256 | AAAATTCGCCAAGCCATC |
| SEQ ID NO. 257 | /5HEX/TGCATAAGC/ZEN/CAGTGCGTTTTTATAT/3IABkFQ/ |
| SEQ ID NO. 258 | GAGAGGATGAYCAGCCACAC |
| SEQ ID NO. 259 | CGCCCATTGTSCAATATTCC |
| SEQ ID NO. 260 | 5TYE665/TGAGACACGGTCCAGACTCCTACG/3IAbRQSp |

The present teachings provide a molecular assay. The present teachings may provide a qualitative (i.e. end point) molecular assay for the detection of family-specific KPC, ESBL, MBL, and ampC gene targets. The present teachings may provide a qualitative (i.e. end point) molecular assay for the detection of family-specific plasmid-mediated ampC β-lactamase genes. The present teachings may provide a qualitative (i.e. end point) molecular assay for the detection of OXA gene targets. Fluorescently-labeled DNA probes may be used for detection. The assay of the present teachings may provide for differentiation between a plasmid-mediated ampC β-lactamase gene from a chromosomal ampC β-lactamase gene; provided the two genes are not from the same chromosomal origin. The assay may involve extraction of DNA from bacterial cells. The assay may include subsequent PCR amplification. The assay may include gel-based detection.

In contrast, to traditional phenotypic methods which require 24-48 hours for data, the present teachings may provide for data generation in just hours or one hour. The total time required for DNA extraction, PCR set-up, amplification, and analysis may be around about 2 hours to about 3 hours. The sensitivity of the assay may be about 100%. The specificity of the assay may be about 100%. Therefore, the present teachings provide for fast and reliable detection. Implementation of such rapid assays have a positive impact for infection control and patient care.

The present teachings allow for the detection of multiple β-lactamase gene families. The β-lactamases may include all major β-lactamases including ampC types. For example, the present teachings may allow for identification of up to six to nine β-lactamase gene families. The β-lactamase gene families may include CMY, CTX-Ms, DHA, IMP, KPC, NDM, OXA and VIM. The AmpC β-lactamases gene families may include MOX, ACC, FOX, DHA, CMY and EBC.

The present teachings provide for a kit which allows for identification of at least nine β-lactamase gene families. The gene families may include: IMP-1-like, NDM-like, OXA-48-like, CTX-M-14-like, CTX-M-15-like, CMY-2-like, DHA-like, VIM-like, and KPC-like. The kit may also include an endogenous internal control (IC) that targets a conserved region common in gram-negative bacteria to reduce false negatives due to PCR inhibition, DNA degradation, or poor extraction. It is contemplated that the endogenous internal control discriminates false negative samples from true negative samples due to but not limited to one or more of PCR inhibition, DNA degradation, and/or poor extraction. The kit may utilize sequence-specific primer pairs for the PCR amplification of each target group. The kit may utilize fluorescently-labeled, target-specific DNA probes for detection by real-time PCR.

The kit may include one or more multiplex primer-probe mixes containing one or more primers and one or more probes. The multiplex primer-probe mix may be a 10× PCR mix. In one example, the kit includes three multiplex primers-probes mix vials. The mix vials may provide for simultaneous real-time PCR amplification of all targets between three reaction tubes. PCR Mix 1 may amplify a first set of three gene families. For example, CMY-2, CTX-M-14, and CTX-M-15. PCR Mix 2 may amplify a second set of three gene families. For example, OXA-48, IMP, and VIM. PCR mix 3 may amplify a third set of gene families. For example, DHA, KPC, and NDM. The multiplex mix may also include an internal control (IC) in each mix. The kit may include three external DNA control vials or first control mix vial, a second control mix vial and a third control mix vial. The DNA control mix vial may contain synthetic DNA templates of the corresponding multiplex targets. The DNA control mixes may serve as a positive control for each multiplex reaction. The DNA control mix may contain stabilized bacteria with chromosomal or transmissible genetic elements in a sample matrix similar to a patient sample.

The present teachings provide for a kit which allows for identification of at least six plasmid-mediated ampC gene families. The gene families may include: MOX-like, DHA-like, ACC-like, EBC-like, FOX-like, and CMY-2-like. The kit may also include an endogenous internal control (IC) that targets a conserved region common in gram-negative bacteria to reduce false negatives due to PCR inhibition, DNA degradation, or poor extraction. It is contemplated that the endogenous internal control discriminates false negative samples from true negative samples due to but not limited to one or more of PCR inhibition, DNA degradation, and/or poor extraction. The kit may utilize sequence-specific primer pairs for the PCR amplification of each family. The kit may utilize fluorescently-labeled, target-specific DNA probes for detection by real-time PCR.

The kit may include one or more multiplex primer-probe mixes containing one or more primers and one or more probes. The multiplex primer-probe mix may be a 10× PCR mix. In one example, the kit includes two multiplex primers-probes mix vials. The mix vials may provide for simultaneous real-time PCR amplification of all targets between two reaction tubes. PCR Mix 1 may amplify a first set of three gene families. For example, MOX, ACC and FOX. PCR Mix 2 may amplify a second set of three gene families. For example, DHA, EBC and CMY-2. The multiplex mix may also include an internal control (IC) in each mix. The kit may include two external DNA control vials or first control mix vial and a second control mix vial. The DNA control mix vial may contain synthetic DNA templates of the corresponding multiplex targets. The DNA control mixes may serve as a positive control for each multiplex reaction.

The present teachings provide for a kit which allows for identification of at least six OXA carbapenemase gene families. The gene families may include: OXA-23, OXA-24/40, OXA-48, OXA-51, OXA-58, and OXA-143. The gene families may include like gene families. The kit may also include an endogenous internal control (IC) that targets a conserved region common in gram-negative bacteria to reduce false negatives due to PCR inhibition, DNA degradation, or poor extraction. It is contemplated that the endogenous internal control discriminates false negative samples from true negative samples due to but not limited to one or more of PCR inhibition, DNA degradation, and/or poor extraction. The kit may utilize sequence-specific primer pairs for the PCR amplification of each family. The kit may utilize fluorescently-labeled, target-specific DNA probes for detection by real-time PCR.

The kit may include one or more multiplex primer-probe mixes containing one or more primers and one or more probes. The multiplex primer-probe mix may be a 10× PCR mix. In one example, the kit includes two multiplex primers-probes mix vials. The mix vials may provide for simultaneous real-time PCR amplification of all targets between two reaction tubes. PCR Mix 1 may amplify a first set of three gene families. For example, OXA 143, OXA 23 and OXA 51. PCR Mix 2 may amplify a second set of three gene families. For example, OXA 24/40, OXA-48 and OXA-58. The multiplex mix may also include an internal control (IC) in each mix. The kit may include two external DNA control vials or first control mix vial and a second control mix vial. The DNA control mix vial may contain synthetic DNA templates of the corresponding multiplex targets. The DNA control mixes may serve as a positive control for each multiplex reaction.

In addition, the present teachings contemplate that the kit or kits of the present teachings may provide for the detection of a non-beta lactamase gene family. The kit or kits may provide for detection of plasmid-mediated mechanisms of antibiotic resistance for one more types/categories of antibiotics. For example, the kit may also provide for the detection of the MCR-1 gene which confers polymixin resistance. The kit or kits may include primer sequences, probe sequences, and a control sequence for detection of one or more non-beta lactamase gene family in addition to beta-lactamase genes. For example, a kit may provide for the detection of ampC genes families and a MCR-1 gene family.

Furthermore, the present teachings allow for the expansion of the detection of other β-lactamase gene families including TEM and SHV. The gene families may include like gene families. The kit may also include an endogenous internal control (IC) that targets a conserved region common in gram-negative bacteria to reduce false negatives due to PCR inhibition, DNA degradation, or poor extraction. It is contemplated that the endogenous internal control discriminates false negative samples from true negative samples due to but not limited to one or more of PCR inhibition, DNA degradation, and/or poor extraction. The kit may utilize sequence-specific primer pairs for the PCR amplification of each family. The kit may utilize fluorescently-labeled, target-specific DNA probes for detection by real-time PCR.

The kit or kits of the present teachings may include synthetic DNA oligonucleotide primers, target-specific DNA probes and DNA controls for the specified gene targets suspended in TE buffer, pH 8.0. The contents of the kit may be enclosed in vials. For example, the one or more 10× PCR mixes may be comprised of 275 μL. For example, the one or more control mixes may be comprised of 14 μL. For example, the contents of the kit may be sufficient for about 100 reactions total and about 12 reactions of the control DNA mix.

Detection of each target is based on the optical fluorescence of the fluorophore conjugated to each target-specific DNA probe. Any suitable fluorophore and nucleic acid sequence combination may be used. For example, the fluorophores may be selected from the group consisting of: FAM, HEX, TEX615 and TYE665.

The present teachings provide assays for the detection of β-lactamase gene families from a biological sample. The assays may be included in a kit or kits. The kit may provide for the detection of β-lactamase by various molecular biology technologies and platforms. The kit may include one or more primers and/or probes for the identification by polymerase chain reaction or microarray of one or more β-lactamase genes selected from the group consisting of: CMY, CTX-M, OXA, IMP, VIM, DHA, KPC, MOX, ACC, FOX, EBC, NDM, TEM, and SHV.

The kit may include one or more primers and/or probes for the identification by polymerase chain reaction or microarray of a non-beta lactamase gene family which confers antibiotic resistance. The kit may include one or more primers and/or probes for the identification by polymerase chain reaction or microarray of one or more MCR genes. The kit may include one or more primers and/or probes for the identification by polymerase chain reaction or microarray of a MCR-1 gene.

The kit may provide for detection of specified targets from crude biological samples such as blood, urine, plasma, feces, sputum, etc. The kit may provide for detection of specified targets directly from or extracted directly from crude biological samples including but not limited to blood, blood cultures, urine, plasma, feces, fecal swabs, peri-rectal/peri-anal swabs, sputum, and bacterial cultures.

The kit may be used for detection of specified targets from purified nucleic acid samples. The kit may be used for any nucleic acid amplification methodology. The kit may be used with conventional polymerase chain reaction. The kit may be used with real-time polymerase chain reaction. The kit may be used with digital droplet polymerase chain reaction. The kit may be used with detection by microarray technology. The kit may be used with fluorescence and/or infra-red probe-based detection chemistries. The kit may be used with intercalating dye-based detection chemistries. The kit may be used for detection of nucleic acid polymerase chain reaction amplicons ranging from 25 base pairs to 2000 base pairs.

The kit may include various reagents. The various reagents may be contained in various vials. The kit may include a primer set or primer sets. The primer set or primer sets may be labeled or unlabeled with a tracking dye or fluorophore. The kit may include probes. The kit may include a primer-probe mix. The kit may include controls. The kit may include magnesium chloride. The kit may include dNTPs. The kit may include DNA polymerase. The kit may include a tracking dye. The kit may include a composition containing a tracking dye. The kit may include a written protocol. The kit may include a customized master mix in a single tube, two tubes, three tubes, or four tubes containing all chemicals and enzymes necessary to run the PCR assay described herein. The kit may include freeze-dried or lyophilized reagents in a single assay tube or multiple assay tubes. The kit may provide for detection of nucleic acid and the kit reagents may be provided in any liquid form, pooled reaction mix, or lyophilized, freeze dried, or cryo-preserved format.

The kit may include a primer set. The primer set may include at least one primer pair. A primer pair may include a forward primer and a reverse primer. The primer set may include one pair of primers. The primer set may include more than one pair of primers. The primer set may include two pairs of primers. The primer set may include three pairs of primers. The primer set may include one to six pairs of primers. The primer set may include one to ten pairs of primers. The primer set may include up to 30 pairs of primers. The primer set may include up to 50 pairs of primers. The primer set may include up to 100 pairs of primers.

The kit may include a primer-probe mix. The primer-probe mix may include a primer set. The primer-probe mix may include one or more probes. Each pair of primers of the primer set may include a probe or set of probes. The primer-probe mix may include a pair of internal control primers. The pair of internal control primers may include a forward primer and a reverse primer. The primer-probe mix may include an internal control probe.

For example, a primer-probe mix may include one or more pairs of primers, one associated probe per primer pair and internal controls including a pair of primers and a probe. Preferably, the primer-probe mix is a multiplex mix including more than one pair of primers, a probe for each primer pair and internal controls. The multiplex mix may be used for the identification of more than one β-lactamase gene family. Each primer pair and probe may detect a different β-lactamase gene family. For example, three primer pairs and their associated three probes may be used for detection of three different β-lactamase gene families.

The DNA concentration range of each primer set in a PCR may be about 1 nM to about 10 μM (10,000 nM). One or more primers may be labeled with a florescent marker as a probe. The DNA concentration of each probe in a PCR may be about 1 nM to about 10,000 nM. The DNA concentration of each probe in a PCR may be about 10 to about 500 nM.

The kit may include at least one control. The kit may include one, two, three or four controls. The kit may include one or more negative controls. The negative control may include nucleic acid known to express a resistance gene other than the target gene of interest. The kit may include one or more positive controls. The one or more positive controls may be internal controls. The positive control may include nucleic acid known to express or contain the resistance gene. The kit may include an endogenous internal control to reduce false negatives due to PCR inhibition, DNA degradation, and/or poor extraction. It is contemplated that the endogenous internal control discriminates false negative samples from true negative samples due to but not limited to one or more of PCR inhibition, DNA degradation, and/or poor extraction. The endogenous internal control may target a conserved nucleotide sequence or sequences common to the Gram-negative bacteria genome. For example, the internal control may detect the 16S rRNA and/or 23S rRNA gene(s). The internal control may detect the 16S and/or 23S rRNA gene for E. Coli, Pseudomonas, Acinetobacter, Klebsiella and Salmonella.

The kit may include control vector in the control vial. One or more μls of the vector control may be added to a 25 μl reaction to get the working concentration. The DNA concentrations for each control vector may be equivalent to 0.1 copy to 2000 copies or 0.0000243 pg/uL to 0.0455 pg/uL. The DNA concentrations for each control vector may be equivalent to 10 copies to 5000 copies or 0.001 pg/uL to 0.5 pg/uL. Control vector concentrations may be as high as 1×10(9) copies and any dilution thereof.

The assays of the present teachings may include the use of magnesium chloride. The kit may include magnesium chloride. The assay may be utilized with a concentration of about 2 mM to about 7 mM MgCl₂. Preferably, the concentration is about 3.0 mM to about 5.5 mM MgCl₂. More preferably, the concentration is 5.0 mM MgCl₂ for an assay for the detection of β-lactamase genes. More preferably the concentration is 5.0 mM MgCl₂ for an assay for the detection of ampC β-lactamase genes. More, preferably, the concentration is 5 mM MgCl₂ for an assay for the detection of OXA genes.

The assays of the present teachings may include the use of DNA polymerase. The kit may include DNA polymerase. The assay may be utilized with a concentration of about 0.25 U/25 ul reaction to about 3 U/25 ul reaction of DNA polymerase. Preferably, the concentration is 1.25 U/25 μl reaction DNA polymerase for an assay for the detection of β-lactamase genes. Preferably the concentration is 1.25 U/25 μl DNA polymerase for an assay for the detection of β-lactamase ampC genes. For example, the present teachings may utilize the PhilisaFAST® DNA polymerase.

The assays and methods of the present teachings may include a PCR cycling protocol. In one example, the cycling protocol comprises (1) 95° C. for 30 s; (2) 95° C. for 1 s; (3) 55° C. for 10 s; (4) 68° C. for 20 s; and repeating steps (2) to (4) for 40 cycles. In one example, the cycling protocol comprises (1) 95° C. for 30 s; (2) 95° C. for 6 s; (3) 66° C. for 10 s; and repeating steps (2) to (3) for 40 cycles. In one example, the cycling protocol includes a hot start of 98° C. for 30 s and 30 cycles of: 98° C. for 5 s, 60° C. for 10 s and 72° C. for 20 s. In one example, the cycling protocol includes using 98° C. for 30 s, followed by 30 cycles of 98° C. for 5 s, 60° C. for 10 s., and 72° C. for 25 s. In one example, the PCR protocols include a detection step where fluorescent signal is measured.

The kit may include one or more of the following: primer, probe and control. A mix of one or more of the following: primer, probe and internal control, may be enclosed in one container. A mix of one or more of the following: primer, probe and internal control, may be enclosed in more than one container. The container may be a vial. In one example, the kit includes 3 DNA control vials and 3 10× primer/probe mix vials. Nine antibiotic resistance gene families and one internal control may be identified with the vials. In one example, the kit includes 2 DNA control vials and 2 10× primer/probe mix vials. Six antibiotic resistance gene families and one internal control may be identified with the vials.

The present teachings allow for detection of the β-lactamase CMY-2 gene family from a biological sample. The present teachings provide for a kit including one or more primers and/or probes for the identification by multiplex real-time polymerase chain reaction of β-lactamase genes including the CMY-2-like gene family. The biological sample may include Gram-negative bacteria such as Escherichia coli, Proteus mirabilis, Klebsiella pneumoniae, Klebsiella oxytoca, Serratia marcescens, Citrobacter freundii and other Citrobacter species. The CMY-2-like genes detected may include CMY-2, CMY-4, CMY-6, CMY-7, CMY-12, CMY-14, CMY-15, CMY-16, CMY-18, CMY-21, CMY-22, CMY-23, CMY-24, CMY-25, CMY-26, CMY-27, CMY-28, CMY-29, CMY-30, CMY-31, CMY-32, CMY-33, CMY-34, CMY-35, CMY-37, CMY-38, CMY-39, CMY-40, CMY-41, CMY-42, CMY-43, CMY-44, CMY-45, CMY-46, CMY-47, CMY-48, CMY-49, CMY-50, CMY-51, CMY-53, CMY-54, CMY-55, CMY-56, CMY-57, CMY-58, CMY-59, CMY-60, CMY-61, CMY-62, CMY-63, CMY-64, CMY-65, CMY-66, CMY-67, CMY-68, CMY-69, CMY-71, CMY-72, CMY-73, CMY-75, CMY-76, CMY-77, CMY-78, CMY-79, CMY-80, CMY-81, CMY-84, CMY-85, CMY-86, CMY-87, CMY-89, CMY-90, CMY-96, CMY-97, CMY-99, CMY-102, CMY-103, CMY-104, CMY-105, CMY-107, CMY-108, CMY-110, CMY-111, CMY-112, CMY-113, CMY-114, CMY-115, CMY-116, CMY-117, CMY-118, CMY-119, CMY-121, CMY-122, CMY-124, CMY-125, CMY-126, CMY-127, CMY-128, CMY-129, CMY-130, CMY-131, CMY-132, CMY-133 and CMY-135.

The present teachings allow for the detection of the β-lactamase CTX-M gene family from a biological sample. The present teachings provide for a kit including one or more primers and/or probes for the identification by multiplex real-time polymerase chain reaction of β-lactamase genes including the CTX-M-14-like gene family. The biological sample may include Gram-negative bacteria such as Klebsiella pneumoniae, Enterobacter cloacae, Escherichia coli, Salmonella enterica, Proteus mirabilis and Shigella species. The CTX-M-14-like genes detected may include CTX-M-9, CTX-M-13, CTX-M-14, CTX-M-16, CTX-M-17, CTX-M-19, CTX-M-21, CTX-M-24, CTX-M-27, CTX-M-38, CTX-M-51, CTX-M-64, CTX-M-65, CTX-M-67, CTX-M-82, CTX-M-83, CTX-M-84, CTX-M-85, CTX-M-86, CTX-M-90, CTX-M-93, CTX-M-98, CTX-M-99, CTX-M-102, CTX-M-104, CTX-M-105, CTX-M-110, CTX-M-111, CTX-M-112, CTX-M-113, CTX-M-121, CTX-M-122, CTX-M-123, CTX-M-125, CTX-M-129, CTX-M-130, CTX-M-132, CTX-M-134, CTX-M-147, CTX-M-148 and CTX-M-159.

The present teachings allow for the detection of the β-lactamase CTX-M gene family from a biological sample. The present teachings provide for a kit including one or more primers and/or probes for the identification by multiplex real-time polymerase chain reaction of β-lactamase genes including the CTX-M-15-like gene family. The biological sample may include Gram-negative bacteria such as Escherichia coli, Klebsiella pneumoniae, Citrobacter freundii, Shigella species and Proteus mirabilis. The CTX-M-15-like genes detected may include CTX-M-1, CTX-M-3, CTX-M-10, CTX-M-15, CTX-M-22, CTX-M-28, CTX-M-29, CTX-M-30, CTX-M-32, CTX-M-37, CTX-M-55, CTX-M-64, CTX-M-71, CTX-M-103, CTX-M-117, CTX-M-123, CTX-M-132, CTX-M-136, CTX-M-138, CTX-M-142, CTX-M-144, CTX-M-155, CTX-M-156, CTX-M-157, CTX-M-158, CTX-M-163, CTX-M-164, CTX-M-166 and CTX-M-172.

The present teachings allow for the detection of the β-lactamase DHA gene family from a biological sample. The present teachings provide for a kit including one or more primers and/or probes for the identification by multiplex real-time polymerase chain reaction of β-lactamase genes including the DHA-like family. The biological sample may include Gram-negative bacteria such as Klebsiella pneumoniae, Morganella morganii, Escherichia coli, Enterobacter cloacae, Proteus mirabilis and Citrobacter koseri. The DHA-like genes detected may include DHA-1, DHA-2, DHA-5, DHA-6, DHA-7, DHA-9, DHA-10, DHA-12, DHA-13, DHA-14, DHA-15, DHA-16, DHA-17, DHA-18, DHA-19, DHA-20, DHA-21 and DHA-22.

The present teachings allow for the detection of the β-lactamase IMP gene family from a biological sample. The present teachings provide for a kit including one or more primers and/or probes for the identification by multiplex real-time polymerase chain reaction of β-lactamase genes including the IMP-like family. The biological sample may include Gram-negative bacteria such as Serratia marcescens, Escherichia coli and Pseudomonas aeruginosa. The IMP-like genes detected may include IMP-1, IMP-2, IMP-3, IMP-4, IMP-5, IMP-6, IMP-7, IMP-8, IMP-9, IMP-10, IMP-13, IMP-14, IMP-15, IMP-16, IMP-18, IMP-19, IMP-20, IMP-22, IMP-24, IMP-25, IMP-26, IMP-27, IMP-28, IMP-30, IMP-32, IMP-33, IMP-34, IMP-37, IMP-38, IMP-40, IMP-42, IMP-45, IMP-48, IMP-49, IMP-51 and IMP-52.

The present teachings allow for the detection of the β-lactamase KPC gene family from a biological sample. The present teachings provide for a kit including one or more primers and/or probes for the identification by multiplex real-time polymerase chain reaction of β-lactamase genes including the KPC-like family. The biological sample may include Gram-negative bacteria such as Klebsiella pneumoniae, Escherichia coli, Enterobacter cloacae and other Enterobacter species, Pseudomonas aeruginosa and Acinetobacter baumannii. The KPC-like genes detected may include KPC-1, KPC-2, KPC-3, KPC-4, KPC-5, KPC-6 KPC-7, KPC-8, KPC-9, KPC-10, KPC-11, KPC-13, KPC-14, KPC-15, KPC-16, KPC-17 KPC-18, KPC-19, KPC-21, KPC-22, KPC-47, KPC-56, KPC-63, KPC-272, KPC-484, KPC-629, KPC-727, and KPC-860.

The present teachings allow for the detection of the β-lactamase NDM gene family from a biological sample. The present teachings provide for a kit including one or more primers and/or probes for the identification by multiplex real-time polymerase chain reaction of β-lactamase genes including the NDM-like family. The biological sample may include Gram-negative bacteria such as Escherichia coli, Acinetobacter baumannii, Enterobacter cloacae and Klebsiella pneumoniae. The NDM-like genes detected may include NDM-1, NDM-2, NDM-3, NDM-4, NDM-5, NDM-6, NDM-7, NDM-8, NDM-9, NDM-10, NDM-11, NDM-12, NDM-13, NDM-15, NDM-16 and NDM-32.

The present teachings allow for the detection of the β-lactamase OXA gene family from a biological sample. The present teachings provide for a kit including one or more primers and/or probes for the identification by multiplex real-time polymerase chain reaction of β-lactamase genes including the OXA-48-like family. The biological sample may include Gram-negative bacteria such as Klebsiella pneumoniae, Enterobacter cloacae, Shewanella xiamenensis, Escherichia coli and Serratia marcescens. The OXA-48-like genes detected may include OXA-48, OXA-162, OXA-163, OXA-181, OXA-199, OXA-204, OXA-232, OXA-244, OXA-245, OXA-247, OXA-370, OXA-405, OXA-416, OXA-438 and OXA-439.

The present teachings provide for a kit including one or more primers and/or probes for the identification by multiplex real-time polymerase chain reaction of β-lactamase genes including one or more of the following: OXA-143-like, OXA-23-like, OXA-51-like, OXA-48-like, OXA-58-like and OXA24/40-like. The OXA-143-like genes detected may include the following: OXA-143, OXA-182, OXA-231, OXA-253, and OXA-255. The OXA-23-like genes detected may include the following: OXA-23, OXA-27, OXA-49, OXA-73, OXA-102, OXA-103, OXA-105, OXA-133, OXA-134, OXA-146, OXA-165, OXA-166, OXA-167, OXA-168, OXA-169, OXA-170, OXA-171, OXA-225 and OXA-239. The OXA-51-like genes detected may include the following: OXA-51, OXA-64, OXA-65, OXA-66, OXA-67, OXA-68, OXA-69, OXA-70, OXA-71, OXA-75, OXA-76, OXA-77, OXA-78, OXA-79, OXA-80, OXA-82, OXA-83, OXA-84, OXA-86, OXA-87, OXA-88, OXA-89, OXA-90, OXA-91, OXA-92, OXA-93, OXA-94 OXA-95, OXA-98, OXA-99, OXA-100, OXA-104, OXA-106, OXA-107, OXA-108, OXA-109, OXA-110, OXA-111, OXA-112, OXA-113, OXA-115, OXA-116, OXA-117, OXA-120, OXA-121, OXA-122, OXA-123, OXA-124, OXA-125, OXA-126, OXA-127, OXA-128, OXA-130, OXA-131, OXA-132, OXA-138, OXA-144, OXA-148, OXA-149, OXA-150, OXA-172, OXA-173, OXA-174, OXA-175, OXA-176, OXA-177, OXA-178, OXA-179, OXA-180, OXA-194, OXA-195, OXA-196, OXA-197, OXA-200, OXA-201, OXA-202, OXA-203, OXA-206, OXA-208, OXA-216, OXA-217, OXA-219, OXA-223, OXA-241, OXA-242, OXA-248, OXA-249, OXA-250 and OXA-254. The OXA-48-like genes detected may include the following: OXA-48, OXA-48b, OXA-162, OXA-163, OXA-181, OXA-199, OXA-204, OXA-232, OXA-244, OXA-245 and OXA-247. The OXA-58-like genes may include the following: OXA-58, OXA-96, OXA-97 and OXA-164. The OXA-40-like genes may include the following: OXA-40, OXA-25, OXA-26, OXA-72, OXA-139, OXA-160 and OXA-207.

The present teachings allow for the detection of the β-lactamase VIM gene family from a biological sample. The present teachings provide for a kit including one or more primers and/or probes for the identification by multiplex real-time polymerase chain reaction of βlactamase genes including the VIM-like family. The biological sample may include Gram-negative bacteria such as Klebsiella oxytoca, Citrobacter freundii, Klebsiella pneumoniae, Pseudomonas aeruginosa, Escherichia coli and Enterobacter cloacae. The VIM-like genes detected may include VIM-1, VIM-2, VIM-3, VIM-4, VIM-5, VIM-6, VIM-8, VIM-9, VIM-10, VIM-11, VIM-12, VIM-13, VIM-14, VIM-15, VIM-16, VIM-17, VIM-18, VIM-19, VIM-20, VIM-23, VIM-24, VIM-25, VIM-26, VIM-27, VIM-28, VIM-31, VIM-33, VIM-34, VIM-35, VIM-36, VIM-37, VIM-38, VIM-39, VIM-40, VIM-41, VIM-42, VIM-43, VIM-44, VIM-45 and VIM-46.

The present teachings allow for the detection of the AmpC β-lactamase MOX gene family from a biological sample. The present teachings provide for a kit including one or more primers and/or probes for the identification by multiplex real-time polymerase chain reaction of β-lactamase genes including the MOX-like family. The biological sample may include Gram-negative bacteria such as Klebsiella pneumoniae, Aeromonas punctata/Aeromonas caviae and other Aeromonas species and Escherichia coli. The MOX-like genes detected may include MOX-1, MOX-2, MOX-3, MOX-4, MOX-5, MOX-6, MOX-7, MOX-8, MOX-10, CMY-1, CMY-8, CMY-9, CMY-10, CMY-11 and CMY-19.

The present teachings allow for the detection of the AmpC β-lactamase ACC gene family from a biological sample. The present teachings provide for a kit including one or more primers and/or probes for the identification by multiplex real-time polymerase chain reaction of β-lactamase genes including the ACC-like family. The biological sample may include Gram-negative bacteria such as Salmonella enterica, Escherichia coli, Hafnia alvei and Proteus mirabilis. The ACC-like genes detected may include ACC-1, ACC-2, ACC-4, ACC-5 and ACC-6.

The present teachings allow for the detection of the AmpC β-lactamase FOX gene family from a biological sample. The present teachings provide for a kit including one or more primers and/or probes for the identification by multiplex real-time polymerase chain reaction of β-lactamase genes including the FOX-like family. The biological sample may include Gram-negative bacteria such as Klebsiella pneumoniae and Aeromonas punctata. The FOX-like genes detected may include FOX-1, FOX-2, FOX-3, FOX-4, FOX-5, FOX-6, FOX-7, FOX-8, FOX-9, FOX-10 and FOX-12.

The present teachings allow for the detection of the AmpC β-lactamase DHA gene family from a biological sample. The present teachings provide for a kit including one or more primers and/or probes for the identification by multiplex real-time polymerase chain reaction of β-lactamase genes including the DHA-like family. The biological sample may include Gram-negative bacteria such as Klebsiella pneumoniae, Morganella morganii, Escherichia coli and Enterobacter cloacae. The DHA-like genes detected may include DHA-1, DHA-2, DHA-5, DHA-6, DHA-7, DHA-9, DHA-10, DHA-12, DHA-13, DHA-14, DHA-15, DHA-16, DHA-17, DHA-18, DHA-19, DHA-20, DHA-21 and DHA-22.

The present teachings allow for the detection of the AmpC β-lactamase CMY-2 gene family from a biological sample. The present teachings provide for a kit including one or more primers and/or probes for the identification by multiplex real-time polymerase chain reaction of β-lactamase genes including the CMY-2-like family. The biological sample may include Gram-negative bacteria such as Klebsiella pneumoniae, Morganella morganii, Escherichia coli and Enterobacter cloacae. The CMY-2-like genes detected include CMY-2, CMY-4, CMY-6, CMY-7, CMY-12, CMY-14, CMY-15, CMY-16, CMY-18, CMY-21, CMY-22, CMY-23, CMY-24, CMY-25, CMY-26, CMY-27, CMY-28, CMY-29, CMY-30, CMY-31, CMY-32, CMY-33, CMY-34, CMY-35, CMY-37, CMY-38, CMY-39, CMY-40, CMY-41, CMY-42, CMY-43, CMY-44, CMY-45, CMY-46 CMY-47, CMY-48, CMY-49, CMY-50, CMY-51, CMY-53, CMY-54, CMY-55, CMY-56, CMY-57, CMY-58, CMY-59, CMY-60, CMY-61, CMY-62, CMY-63, CMY-64, CMY-65, CMY-66, CMY-67, CMY-68, CMY-69, CMY-71, CMY-72, CMY-73, CMY-75, CMY-76, CMY-77, CMY-78, CMY-79, CMY-80, CMY-81, CMY-84, CMY-85 CMY-86, CMY-87, CMY-89, CMY-90, CMY-96, CMY-97, CMY-99, CMY-102, CMY-103, CMY-104 CMY-105, CMY-107, CMY-108, CMY-110, CMY-111, CMY-112, CMY-113, CMY-114, CMY-115 CMY-116, CMY-117, CMY-118, CMY-119, CMY-121, CMY-122, CMY-124, CMY-125, CMY-126, CMY-127, CMY-128, CMY-129, CMY-130, CMY-131 CMY-132, CMY-133 and CMY-135.

The present teachings allow for the detection of the AmpC β-lactamase EBC gene family from a biological sample. The present teachings provide for a kit including one or more primers and/or probes for the identification by multiplex real-time polymerase chain reaction of β-lactamase genes including the EBC-like family such as ACT and MIR. The biological sample may include Gram-negative bacteria such as Enterobacter cloacae, Klebsiella pneumoniae, Enterobacter asburiae, Enterobacter kobei, and other Enterobacter species. The EBC-like genes detected may include ACT-1, ACT-2, ACT-5, ACT-8, ACT-13, ACT-14, ACT-15, ACT-16, ACT-17, ACT-18, ACT-20, ACT-21, ACT-23, ACT-24, ACT-25, ACT-27, ACT-29, ACT-30, ACT-31, ACT-32, ACT-33, ACT-34, ACT-35, ACT-36, ACT-37, ACT-38, MIR-1, MIR-2, MIR-3, MIR-4, MIR-6, MIR-7, MIR-8, MIR-9, MIR-10, MIR-11, MIR-12, MIR-13, MIR-14, MIR-15, MIR-16, MIR-17 and MIR-18.

The present teachings may allow for the detection of the β-lactamase TEM gene family from a biological sample. The present teachings provide for a kit including one or more primers and/or probes for the identification by multiplex real-time polymerase chain reaction of β-lactamase genes including the TEM-like family. The biological sample may include Gram-negative bacteria such as Klebsiella pneumoniae, Enterobacter cloacae, Shewanella xiamenensis, Escherichia coli and Serratia marcescens. The TEM-like genes detected may include TEM-1, TEM-2, TEM-3, TEM-15, TEM-20, TEM-32, TEM-40, TEM-52, TEM-88, TEM-91, TEM-97, TEM-98, TEM-106, TEM-107, TEM-112, TEM-120, TEM-126, TEM-135, TEM-141, TEM-150, TEM-153, TEM-163, TEM-168, TEM-170, TEM-171, TEM-206, TEM-214, and TEM-220.

The present teachings may allow for the detection of the β-lactamase SHV gene family from a biological sample. The present teachings provide for a kit including one or more primers and/or probes for the identification by multiplex real-time polymerase chain reaction of β-lactamase genes including the SHV-like family. The biological sample may include Gram-negative bacteria such as Klebsiella pneumoniae, Enterobacter cloacae, Shewanella xiamenensis, Escherichia coli and Serratia marcescens. The SHV-like genes detected may include SVH-1, SHV-2, SHV-3, SHV-5, SHV-7, SHV-8, SHV-9, SHV-11, SHV-12, SHV-13, SHV-14, SHV-15, SHV-16, SHV-18, SHV-24, SHV-25, SHV-26, SHV-27, SHV-28, SHV-29, SHV-30, SHV-31, SHV-32, SHV-33, SHV-34, SHV-35, SHV-36, SHV-37, SHV-38, SHV-40, SHV-41, SHV-42, SHV-43, SHV-44, SHV-45, SHV-46, SHV-48, SHV-49, SHV-50, SHV-51, SHV-52, SHV-53, SHV-55, SHV-56, SHV-57, SHV-59, SHV-60, SHV-61, SHV-62, SHV-63, SHV-64, SHV-65, SHV-66, SHV-67, SHV-69, SHV-70, SHV-71, SHV-72, SHV-73, SHV-74, SHV-75, SHV-76, SHV-77, SHV-78, SHV-79, SHV-80, SHV-81, SHV-82, SHV-85, SHV-86, SHV-89, SHV-92, SHV-93, SHV-94, SHV-95, SHV-96, SHV-97, SHV-98, SHV-99, SHV-100, SHV-101, SHV-102, SHV-103, SHV-104, SHV-105, SHV-106, SHV-107, SHV-109, SHV-110, SHV-111, SHV-119, SHV-120, SHV-121, SHV-122, SHV-123, SHV-124, SHV-125, SHV-126, SHV-127, SHV-128, SHV-129, SHV-132, SHV-133, SHV-134, SHV-135, SHV-136, SHV-137, SHV-140, SHV-141, SHV-142, SHV-143, SHV-144, SHV-145, SHV-146, SHV-147, SHV-148, SHV-149, SHV-150, SHV-151, SHV-152, SHV-153, SHV-154, SHV-155, SHV-156, SHV-157, SHV-158, SHV-159, SHV-160, SHV-161, SHV-162, SHV-163, SHV-164, SHV-165, SHV-168, SHV-172, SHV-173, SHV-178, SHV-179, SHV-180, SHV-182, SHV-183, SHV-185, SHV-186, SHV-187, SHV-188, SHV-189, SHV-190, SHV-191, SHV-193, SHV-194, SHV-195, SHV-196, and SHV-197.

The present teachings may allow for the detection of the MCR gene family from a biological sample. The present teachings provide for a kit including one or more primers and/or probes for the identification by multiplex real-time polymerase chain reaction of MCR genes including the MCR-like family. The MCR-like genes detected may include MCR-1, MCR-1.2, MCR-1.3, MCR-1.4, MCR-1.5, MCR-1.6, MCR-1.7, MCR-1.8, MCR-1.9 and MCR-2.

The kit of the present teachings may include a mix of at least one primer and/or at least one probe. Primers and/or probes may be degenerate at any nucleotide position. Primers and/or probes may not be degenerate at any nucleotide position. A hydrolysis and/or hybridization probe may be designed for the detection of a specific nucleic acid sequence. Multiple probes may be labeled with a different colored fluorophore. The probe may be labeled with a fluorescent tag at one end and a fluorescent quencher at the other end. Two fluorescent quenchers may be included at one end or within the probe sequence. For example, the fluorophores may be selected from the group consisting of fluorescein, hexachlorofluorescein, TEX 615, and TYE™ 665. The fluorophores may excite between 450 nm and 763 nm and emit between 500 nm and 800 nm. For example, the quenchers may be selected from the group consisting of Iowa Black® quenchers and Black Hole Quenchers®. Peak absorbance of each quencher may be at 531 nm, 534 nm, 578 nm, or 656 nm.

Multiple hydrolysis and/or hybridization probes can be added to the same nucleic acid amplification reaction. The selection of the fluorescent labels may depend on the type of hydrolysis and/or hybridization probe used, the number of targets to be detected and the type of thermal cycler used. Preferable combinations of fluorophores and quenchers for multiplex reactions require appropriate excitation wavelengths and little to no overlap in their emission spectra as well as reduction of background fluorescence. It is contemplated that the probe sequences of the present teachings may be labeled with any suitable fluorophore and quencher combinations. For example, any fluorophore of the present teachings may be attached to any probe DNA sequence of the present teachings.

The one or more primers and/or probes maybe selected from the group consisting of: TGGCCAGAACTGACAGGCAAA, TTTCTCCTGAACGTGGCTGGC, 56-FAM/ACGCTAACT/ZEN/CCAGCATTGGTCTGT/3IABkFQ/, CCGTCACGCTGTTGTTAGG, GCTGTGTTAATCAATGCCACAC, 5HEX/AACTTGCCG/ZEN/AATTAGAGCRGCAGT/3IABkFQ, CGTTTCGTCTGGATCGCAC, GCTGGGTAAAATAGGTCACC, 5TEX615/TATCATTGGTGGTGCCGTAGTCGC/3IAbRQSp, GAGAGGATGAYCAGCCACAC, CGCCCATTGTSCAATATTCC, 5TYE665/TGAGACACGGTCCAGACTCCTACG/3IAbRQSp, AATCACAGGGCGTAGTTGTG, ACCCACCAGCCAATCTTAGG, 56-FAM/TAGCTTGAT/ZEN/CGCCCTCGATTTGGG/3IABkFQ/, GCGGAGTTAACTATTGGCTAG, GGCCAAGCTTCTATATTTGCG, 5HEX/TTRTTYGGT/ZEN/GGTTGYTTTRTTAA/3IABkFQ, GCGGAGTTARYTATTGGCTAG, GGCCAAGCYTCTAWATTTGCG, /5HEX/CCGGACGGT/ZEN/CTTGGTAATTTGGGT/3IABkFQ/, /5HEX/CCGTACGGT/ZEN/TTAGGCAATTTGGGT/3IABkFQ/, GGCGGCGTTGATGTCCTTCG, CCATTCAGCCAGATCGGCATC, 5TEX615/AGCTCTTCTATCCTGGTGCTGCG/3IAbRQSp, AACTTTCACAGGTGTGCTGGGT, CCGTACGCATACTGGCTTTGC, 56-FAM/AAACCGGGC/ZEN/GATATGCGTCTGTAT/3IABkFQ/, GTATCGCCGTCTAGTTCTGC, CCTTGAATGAGCTGCACAGTGG, 5HEX/TCGTCGCGG/ZEN/AACCATTCGCTAAA/3IABkFQ/, GTTTGATCGTCAGGGATGGC, GGCGAAAGTCAGGCTGTG, 5TEX615/CATCAGGACAAGATGGGCGGTATG/3IAbRQSp, GCTGCTCAAGGAGCACAGGAT, CACATTGACATAGGTGTGGTGC, 56-FAM/AGGATGGCA/ZEN/AGGCCCACTATTTCA/3IABkFQ, AACAGCCTCAGCAGCCGGTTA, TTCGCCGCAATCATCCCTAGC, 5HEX/AGCCATTAC/ZEN/GTTCCAGAGTTGCGT/3IABkFQ, GCCGAGGCTTACGGGATCAAG, CAAAGCGCGTAACCGGATTGG, 5TEX615/TCTGCTGAAGTTTRYCGAGGCMAA/3IAbRQSp, AACTTTCACAGGTGTGCTGGGT, CCGTACGCATACTGGCTTTGC, 56-FAM/AAACCGGGC/ZEN/GATATGCGTCTGTAT/3IABkFQ/, CTGGGTTCTATAAGTAAAACCTTCACCGG, CTTCCACTGCGGCTGCCAGTT, 5HEX/GATGCCATT/ZEN/GCYCGSGGTGAAAT/3IABkFQ, CCGAAGCCTATGGCGTGAAATCC, GCAATGCCCTGCTGGAGCG, 5TEX615/ATGTTGGCCTGAACCCAGCG/3IAbRQSp. Primers and/or probes included in this group may or may not be degenerate at any nucleotide position. [SEQ. ID NOS 67-118]

The kit may include one or more primer-probe multiplex mixes. The primer-probe multiplex mix may include one or more internal controls. The primer-probe multiplex mix and one or more internal controls may be enclosed in one container, such as a vial. The primer-probe multiplex mix and one or more internal controls may be enclosed in more than one container, such as vials.

A primer-probe mix may include sequences for detecting any combination of the following genes: CMY-2-like, CTX-M-14-like, CTX-M-15-like, IMP-like, VIM-like, DHA-like, KPC-like, NDM-like, MOX-like, ACC-like, FOX-like, DHA-like, EBC-like, OXA-143-like, OXA-23-like, OXA-51-like, OXA-48-like, OXA-58-like and OXA-24/40-like.

For example, the kit may include a first primer-probe mix and one or more internal controls in a first vial and a second primer-probe mix and one or more internal controls in a second vial. For example, the kit may include a first primer-probe mix and one or more internal controls in a first vial, a second primer-probe mix and one or more internal controls in a second vial and a third primer-probe mix and one or more internal controls in a third vial. Each vial may contain different mixtures. Each vial may contain the same mixture.

The kit may include at least one control DNA mix. The kit may include one or more DNA control mixes. The kit may include exactly two control DNA mixes. The kit may include exactly three control DNA mixes. The DNA control mix may include at least one DNA sequence corresponding to at least one gene family and at least one internal control DNA sequence. The DNA control mix may be enclosed in one container, such as a vial. The DNA control mix may be enclosed in more than one container, such as vials.

For example, the kit may include a first DNA control mix in a first vial and a second DNA control mix in a second vial. For example, the kit may include a first DNA control mix in a first vial, a second DNA control mix in a second vial and a third DNA control mix in a third vial. Each vial may contain different mixtures. Each vial may contain the same mixture.

In one example, the kit includes three primer-probe multiplex mix vials including internal controls and three DNA control mix vials. The three primer-probe multiplex mixes may provide for identification of up to nine antibiotic resistance genes and internal controls. A first primer-probe mix may include sequences for detecting gene families which are CMY-2-like, CTX-M-14-like, CTX-M-15-like and internal controls. A second primer-probe mix may include sequences for detecting gene families which are OXA-48-like, IMP-like, VIM-like and internal controls. A third primer-probe mix may include sequences for detecting gene families which are DHA-like, KPC-like, NDM-like and internal controls. The one or more DNA control mixes may be plasmid or vector controls. A first DNA control mix may include DNA sequences for CMY-2, CTX-M-14, CTX-M-15 and an internal control DNA sequence. A second DNA control mix may include DNA sequences for OXA-48, IMP, VIM and an internal control DNA sequence. A third DNA control mix may include DNA sequences for DHA, KPC, NDM and an internal control DNA sequence.

It is contemplated that the combination of gene families may vary. For example, a primer-probe mix may include sequences for detecting any combination of the following genes: CMY-2-like, CTX-M-14-like, CTX-M-15-like, and OXA-48-like, IMP-like, VIM-like, DHA-like, KPC-like and NDM-like. It is further contemplated that additional β-lactamase gene targets may be included in the primer-probe mix or mixes.

The first primer-probe mix may include one or more primers and/or probes selected from the group consisting of: TGGCCAGAACTGACAGGCAAA, TTTCTCCTGAACGTGGCTGGC, 56FAM/ACGCTAACT/ZEN/CCAGCATTGGTCTGT/3IABkFQ/, CCGTCACGCTGTTGTTAGG, GCTGTGTTAATCAATGCCACAC, 5HEX/AACTTGCCG/ZEN/AATTAGAGCRGCAGT/3IABkFQ, CGTTTCGTCTGGATCGCAC, GCTGGGTAAAATAGGTCACC and 5TEX615/TATCATTGGTGGTGCCGTAGTCGC/3IAbRQSp. The first primer-probe mix may include one or more internal controls selected from the group consisting of: GAGAGGATGAYCAGCCACAC, CGCCCATTGTSCAATATTCC and 5TYE665/TGAGACACGGTCCAGACTCCTACG/3IAbRQSp. A primer-probe mix may include a combination of the one or more said group of primers and/or probes and the one or more said group of internal controls. The primer-probe mix including internal controls may be a multiplex mix. [SEQ. ID NOS 152-163]

The kit may include a first, second and third primer and/or probe mix, the first primer and/or probe mix including one or more primers and/or probes selected from the group consisting of: TGGCCAGAACTGACAGGCAAA, TTTCTCCTGAACGTGGCTGGC, 56-FAM/ACGCTAACT/ZEN/CCAGCATTGGTCTGT/3IABkFQ/, CCGTCACGCTGTTGTTAGG, GCTGTGTTAATCAATGCCACAC, 5HEX/AACTTGCCG/ZEN/AATTAGAGCRGCAGT/3IABkFQ, CGTTTCGTCTGGATCGCAC, GCTGGGTAAAATAGGTCACC, 5TEX615/TATCATTGGTGGTGCCGTAGTCGC/3IAbRQSp, GAGAGGATGAYCAGCCACAC, CGCCCATTGTSCAATATTCC, and 5TYE665/TGAGACACGGTCCAGACTCCTACG/3IAbRQSp. Primers and/or probes included in this group may or may not be degenerate at any nucleotide position. [SEQ. ID NOS 152-163]

The second primer-probe mix may include one or more primers and/or probes selected from the group consisting of: AATCACAGGGCGTAGTTGTG, ACCCACCAGCCAATCTTAGG, 56-FAM/TAGCTTGAT/ZEN/CGCCCTCGATTTGGG/3IABkFQ/, GCGGAGTTAACTATTGGCTAG, GGCCAAGCTTCTATATTTGCG, 5HEX/TTRTTYGGT/ZEN/GGTTGYTTTRTTAA/3IABkFQ, GCGGAGTTARYTATTGGCTAG, GGCCAAGCYTCTAWATTTGCG, /5HEX/CCGGACGGT/ZEN/CTTGGTAATTTGGGT/3IABkFQ/, /5HEX/CCGTACGGT/ZEN/TTAGGCAATTTGGGT/3IABkFQ, GGCGGCGTTGATGTCCTTCG, CCATTCAGCCAGATCGGCATC and 5TEX615/AGCTCTTCTATCCTGGTGCTGCG/3IAbRQSp. The second primer-probe mix may include one or more internal controls selected from the group consisting of: GAGAGGATGAYCAGCCACAC, CGCCCATTGTSCAATATTCC, and 5TYE665/TGAGACACGGTCCAGACTCCTACG/3IAbRQSp. A primer-probe mix may include a combination of the one or more said group of primers and/or probes and the one or more said group of internal controls. The primer-probe mix including internal controls may be a multiplex mix. [SEQ. ID NOS 164-179]

The kit may include a first, second, and third primer and/or probe mix, the second primer and/or probe mix including one or more primers and/or probes selected from the group consisting of: AATCACAGGGCGTAGTTGTG, ACCCACCAGCCAATCTTAGG, 56-FAM/TAGCTTGAT/ZEN/CGCCCTCGATTTGGG/13IABkFQ/, GCGGAGTTAACTATTGGCTAG, GGCCAAGCTTCTATATTTGCG, 5HEX/TTRTTYGGT/ZEN/GGTTGYTTTRTTAA/3IABkFQ, GCGGAGTTARYTATTGGCTAG, GGCCAAGCYTCTAWATTTGCG, /5HEX/CCGGACGGT/ZEN/CTTGGTAATTTGGGT/3IABkFQ/, /5HEX/CCGTACGGT/ZEN/TTAGGCAATTTGGGT/3IABkFQ, GGCGGCGTTGATGTCCTTCG, CCATTCAGCCAGATCGGCATC, 5TEX615/AGCTCTTCTATCCTGGTGCTGCG/3IAbRQSp, GAGAGGATGAYCAGCCACAC, CGCCCATTGTSCAATATTCC, and 5TYE665/TGAGACACGGTCCAGACTCCTACG/3IAbRQSp. Primers and/or probes included in this group may or may not be degenerate at any nucleotide position. [SEQ. ID NOS 164-179]

The third primer-probe mix may include one or more primers and/or probes selected from the group consisting of: AACTTTCACAGGTGTGCTGGGT, CCGTACGCATACTGGCTTTGC, 56-FAM/AAACCGGGC/ZEN/GATATGCGTCTGTAT/3IABkFQ/, GTATCGCCGTCTAGTTCTGC, CCTTGAATGAGCTGCACAGTGG, 5H EX/TCGTCGCGG/ZEN/AACCATTCGCTAAA/3IABkFQ/, GTTTGATCGTCAGGGATGGC, GGCGAAAGTCAGGCTGTG and 5TEX615/CATCAGGACAAGATGGGCGGTATG/3IAbRQSp. The third primer-probe mix may include one or more internal controls selected from the group consisting of: GAGAGGATGAYCAGCCACAC, CGCCCATTGTSCAATATTCC and 5TYE665/TGAGACACGGTCCAGACTCCTACG/3IAbRQSp. A primer-probe mix may include a combination of the one or more said group of primers and/or probes and the one or more said group of internal controls. The primer-probe mix including internal controls may be a multiplex mix. [SEQ. ID NOS 180-191]

The kit may include a first, second and third primer and/or probe mix, the third primer and/or probe mix including one or more primers and/or probes selected from the group consisting of: AACTTTCACAGGTGTGCTGGGT, CCGTACGCATACTGGCTTTGC, 56-FAM/AAACCGGGC/ZEN/GATATGCGTCTGTAT/3IABkFQ/, GTATCGCCGTCTAGTTCTGC, CCTTGAATGAGCTGCACAGTGG, 5HEX/TCGTCGCGG/ZEN/AACCATTCGCTAAA/3IABkFQ/, GTTTGATCGTCAGGGATGGC, GGCGAAAGTCAGGCTGTG, 5TEX615/CATCAGGACAAGATGGGCGGTATG/3IAbRQSp, GAGAGGATGAYCAGCCACAC, CGCCCATTGTSCAATATTCC, and 5TYE665/TGAGACACGGTCCAGACTCCTACG/3IAbRQSp. Primers and/or probes included in this group may or may not be degenerate at any nucleotide position. [SEQ. ID NOS 180-191]

A first DNA control mix may include one or more sequences selected from the group consisting of: TGGCCAGAACTGACAGGCAAACAGTGGCAGGGTATCCGCCTGCTGCACTTAGCCA CCTATACGGCAGGCGGCCTACCGCTGCAGATCCCCGATGACGTTAGGGATAAAGC CGCATTACTGCATTTTTATCAAAACTGGCAGCCGCAATGGACTCCGGGCGCTAAGC GACTTTACGCTAACTCCAGCATTGGTCTGTTTGGCGCGCTGGCGGTGAAACCCTC AGGAATGAGTTACGAAGAGGCAATGACCAGACGCGTCCTGCAACCATTAAAACTG GCGCATACCTGGATTACGGTTCCGCAGAACGAACAAAAAGATTATGCCTGGGGCT ATCGCGAAGGGAAGCCCGTACACGTTTCTCCGGGACAACTTGACGCCGAAGCCTA TGGCGTGAAATCCAGCGTTATTGATATGGCCCGCTGGGTTCAGGCCAACATGGAT GCCAGCCACGTTCAGGAGAAA, CCGTCACGCTGTTGTTAGGAAGTGTGCCGCTGTATGCGCAAACGGCGGACGTACA GCAAAAACTTGCCGAATTAGAGCGGCAGTCGGGAGGCAGACTGGGTGTGGCATT GATTAACACAGC, and CGTTTCGTCTGGATCGCACTGAACCTACGCTGAATACCGCCATTCCCGGCGACCC GAGAGACACCACCACGCCGCGGGCGATGGCGCAGACGTTGCGTCAGCTTACGCT GGGTCATGCGCTGGGCGAAACCCAGCGGGCGCAGTTGGTGACGTGGCTCAAAGG CAATACGACCGGCGCAGCCAGCATTCGGGCCGGCTTACCGACGTCGTGGACTGT GGGTGATAAGACCGGCAGCGGCGACTACGGCACCACCAATGATATTGCGGTGATC TGGCCGCAGGGTCGTGCGCCGCTGGTTCTGGTGACCTATTTTACCCAGC. The first DNA control mix may include the following internal control sequence: GAGAGGATGACCAGCCACACTGGAACTGAGACACGGTCCAGACTCCTACGGGAG GCAGCAGTGGGGAATATTGCACAATGGGCG. A DNA control mix may include a combination of the one or more said group of sequences and the said internal control sequence. A DNA control mix may include any combination of sequences from the first control mix, the second control mix, the third control mix and the internal control sequence. [SEQ. ID NOS 261-264]

A second DNA control mix may include one or more sequences selected from the group consisting of: AATCACAGGGCGTAGTTGTGCTCTGGAATGAGAATAAGCAGCAAGGATTTACCAAT AATCTTAAACGGGCGAACCAAGCATTTTTACCCGCATCTACCTTTAAAATTCCCAAT AGCTTGATCGCCCTCGATTTGGGCGTGGTTAAGGATGAACACCAAGTCTTTAAGTG GGATGGACAGACGCGCGATATCGCCACTTGGAATCGCGATCATAATCTAATCACC GCGATGAAATATTCAGTTGTGCCTGTTTATCAAGAATTTGCCCGCCAAATTGGCGA GGCACGTATGAGCAAGATGCTACATGCTTTCGATTATGGTAATGAGGACATTTCGG GCAATGTAGACAGTTTCTGGCTCGACGGTGGTATTCGAATTTCGGCCACGGAGCA AATCAGCTTTTTAAGAAAGCTGTATCACAATAAGTTACACGTATCGGAGCGCAGCC AGCGTATTGTCAAACAAGCCATGCTGACCGAAGCCAATGGTGACTATATTATTCGG GCTAAAACTGGATACTCGACTAGAATCGAACCTAAGATTGGCTGGTGGGT, GCGGAGTTAGTTATTGGCTAGTTAAAAATAAAATTGAAGTTTTTTATCCCGGCCCGG GGCACACTCAAGATAACGTAGTGGTTTGGTTACCTGAAAAGAAAATTTTATTCGGT GGTTGTTTTGTTAAACCGGACGGTCTTGGTAATTTGGGTGACGCAAATTTAGAAGC TTGGCC and GGCGGCGTTGATGTCCTTCGGGCGGCTGGGGTGGCAACGTACGCATCACCGTCG ACACGCCGGCTAGCCGAGGTAGAGGGGAACGAGATTCCCACGCACTCTCTAGAA GGACTCTCATCGAGCGGGGACGCAGTGCGCTTCGGTCCAGTAGAACTCTTCTATC CTGGTGCTGCGCATTCGACCGACAACTTAGTTGTGTACGTCCCGTCTGCGAGTGT GCTCTATGGTGGTTGTGCGATTCATGAGTTGTCACGCACGTCTGCGGGGAACGTG GCCGATGCCGATCTGGCTGAATGG. The second DNA control mix may include the following internal control sequence: GAGAGGATGACCAGCCACACTGGAACTGAGACACGGTCCAGACTCCTACGGGAG GCAGCAGTGGGGAATATTGCACAATGGGCG. A DNA control mix may include a combination of the one or more said group of sequences and the said internal control sequence. A DNA control mix may include any combination of sequences from the first control mix, the second control mix, the third control mix and the internal control sequence. [SEQ. ID NOS 265-268]

A third DNA control mix may include one or more sequences selected from the group consisting of: AACTTTCACAGGTGTGCTGGGTGCGGTTTCTGTGGCGAAAAAAGAGATGGCGCTG AATGATCCGGCGGCAAAATACCAGCCGGAGCTGGCTCTGCCGCAGTGGAAGGGG ATCACATTGCTGGATCTGGCTACCTATACCGCAGGCGGACTGCCGTTACAGGTGC CGGATGCGGTAAAAAGCCGTGCGGATCTGCTGAATTTCTATCAGCAGTGGCAGCC GTCCCGGAAACCGGGCGATATGCGTCTGTATGCAAACAGCAGTATCGGCCTGTTT GGTGCTCTGACCGCAAACGCGGCGGGGATGCCGTATGAGCAGTTGCTGACTGCA CGGATCCTGGCACCGCTGGGGTTATCTCACACCTTTATTACTGTGCCGGAAAGTG CGCAAAGCCAGTATGCGTACGG, GTATCGCCGTCTAGTTCTGCTGTCTTGTCTCTCATGGCCGCTGGCTGGCTTTTCTG CCACCGCGCTGACCAACCTCGTCGCGGAACCATTCGCTAAACTCGAACAGGACTT TGGCGGCTCCATCGGTGTGTACGCGATGGATACCGGCTCAGGCGCAACTGTAAGT TACCGCGCTGAGGAGCGCTTCCCACTGTGCAGCTCATTCAAGG and GTTTGATCGTCAGGGATGGCGGCCGCGTGCTGGTGGTCGATACCGCCTGGACCG ATGACCAGACCGCCCAGATCCTCAACTGGATCAAGCAGGAGATCAACCTGCCGGT CGCGCTGGCGGTGGTGACTCACGCGCATCAGGACAAGATGGGCGGTATGGACGC GCTGCATGCGGCGGGGATTGCGACTTATGCCAATGCGTTGTCGAACCAGCTTGCC CCGCAAGAGGGGATGGTTGCGGCGCAACACAGCCTGACTTTCGCC. The third DNA control mix may include the following internal control sequence: GAGAGGATGACCAGCCACACTGGAACTGAGACACGGTCCAGACTCCTACGGGAG GCAGCAGTGGGGAATATTGCACAATGGGCG. A DNA control mix may include a combination of the one or more said group of sequences and the said internal control sequence. A DNA control mix may include any combination of sequences from the first control mix, the second control mix, the third control mix and the internal control sequence. [SEQ. ID NOS 269-272]

In one example, the kit includes two primer-probe multiplex mix vials including internal controls and two DNA control mix vials. The two primer-probe multiplex mixes may provide for identification of up to six antibiotic resistance genes and internal controls. A first primer-probe mix may include sequences for detecting gene families which are MOX-like, ACC-like, FOX-like and internal controls. A second primer-probe mix may include sequences for detecting gene families which are DHA-like, ACT/MIR-like, CMY-2-like and internal controls. A first DNA control mix may include DNA sequences for MOX, ACC, FOX and an internal control DNA sequence. A second DNA control mix may include DNA sequences for DHA, ACT/MIR, CMY-2 and an internal control DNA sequence.

It is contemplated that the combination of gene families may vary. For example, a primer-probe mix may include sequences for detecting any combination of the following genes: MOX-like, ACC-like, FOX-like, DHA-like, ACT/MIR-like and CMY-2-like. It is further contemplated that additional β-lactamase gene targets may be included in the primer-probe mix or mixes.

The first primer-probe mix may include one or more primers and/or probes selected from the group consisting of: GCTGCTCAAGGAGCACAGGAT, CACATTGACATAGGTGTGGTGC, 56-FAM/AGGATGGCA/ZEN/AGGCCCACTATTTCA/3IABkFQ, AACAGCCTCAGCAGCCGGTTA, TTCGCCGCAATCATCCCTAGC, 5HEX/AGCCATTAC/ZEN/GTTCCAGAGTTGCGT/3IABkFQ, GCCGAGGCTTACGGGATCAAG, CAAAGCGCGTAACCGGATTGG and 5TEX615/TCTGCTGAAGTTTRYCGAGGCMAA/3IAbRQSp. The first primer-probe mix may include one or more internal controls selected from the group consisting of: GAGAGGATGAYCAGCCACAC, CGCCCATTGTSCAATATTCC and 5TYE665/TGAGACACGGTCCAGACTCCTACG/3IAbRQSp. A primer-probe mix may include a combination of the one or more said group of primers and/or probes and the one or more said group of internal controls. The primer-probe mix including internal controls may be a multiplex mix. [SEQ. ID NOS 192-203]

The second primer-probe mix may include one or more primers and/or probes selected from the group consisting of: AACTTTCACAGGTGTGCTGGGT, CCGTACGCATACTGGCTTTGC, 56-FAM/AAACCGGGC/ZEN/GATATGCGTCTGTAT/3IABkFQ, CTGGGTTCTATAAGTAAAACCTTCACCGG, CTTCCACTGCGGCTGCCAGTT, 5HEX/GATGCCATT/ZEN/GCYCGSGGTGAAAT/3IABkFQ, CCGAAGCCTATGGCGTGAAATCC, GCAATGCCCTGCTGGAGCG, and 5TEX615/ATGTTGGCCTGAACCCAGCG/3IAbRQSp. The second primer-probe mix may include one or more internal controls selected from the group consisting of: GAGAGGATGAYCAGCCACAC, CGCCCATTGTSCAATATTCC and 5TYE665/TGAGACACGGTCCAGACTCCTACG/3IAbRQSp. A primer-probe mix may include a combination of the one or more said group of primers and/or probes and the one or more said group of internal controls. The primer-probe mix including internal controls may be a multiplex mix. [SEQ. ID NOS 204-215]

The kit may include exactly two primer and/or probe mixes, a first primer and/or probe mix including one or more primers and/or probes selected from the group consisting of: GCTGCTCAAGGAGCACAGGAT, CACATTGACATAGGTGTGGTGC, 56-FAM/AGGATGGCA/ZEN/AGGCCCACTATTTCA/3IABkFQ, AACAGCCTCAGCAGCCGGTTA, TTCGCCGCAATCATCCCTAGC, 5HEX/AGCCATTAC/ZEN/GTTCCAGAGTTGCGT/3IABkFQ, GCCGAGGCTTACGGGATCAAG, CAAAGCGCGTAACCGGATTGG, 5TEX615/TCTGCTGAAGTTTRYCGAGGCMAA/3IAbRQSp, GAGAGGATGAYCAGCCACAC, CGCCCATTGTSCAATATTCC, and 5TYE665/TGAGACACGGTCCAGACTCCTACG/3IAbRQSp; and a second primer and/or probe mix including one or more primers and/or probes selected from the group consisting of: AACTTTCACAGGTGTGCTGGGT, CCGTACGCATACTGGCTTTGC, 56-FAM/AAACCGGGC/ZEN/GATATGCGTCTGTAT/3IABkFQ, CTGGGTTCTATAAGTAAAACCTTCACCGG, CTTCCACTGCGGCTGCCAGTT, 5HEX/GATGCCATT/ZEN/GCYCGSGGTGAAAT/3IABkFQ, CCGAAGCCTATGGCGTGAAATCC, GCAATGCCCTGCTGGAGCG, 5TEX615/ATGTTGGCCTGAACCCAGCG/3IAbRQSp, GAGAGGATGAYCAGCCACAC, CGCCCATTGTSCAATATTCC, and 5TYE665/TGAGACACGGTCCAGACTCCTACG/3IAbRQSp. Primers and/or probes included in this group may or may not be degenerate at any nucleotide position. [SEQ. ID NOS 192-215]

A first DNA control mix may include one or more sequences selected from the group consisting of: GCTGCTCAAGGAGCACAGGATCCCGGGCATGGCGGTGGCCGTGCTCAAGGATGG CAAGGCCCACTATTTCAATTACGGGGTGGCCAACCGGGAGAGCGGGGCCAGCGT CAGCGAGCAGACCCTGTTCGAGATAGGATCCGTGAGCAAGACCCTGACTGCGACC CTGGGGGCCTATGCGGTGGTCAAGGGAGCGATGCAGCTGGATGACAAGGCGAGC CGGCACGCGCCCTGGCTCAAGGGATCCGTCTTTGACAGCATCACCATGGGGGAG CTTGCCACCTACAGCGCCGGAGGCCTGCCACTGCAATTCCCCGAGGAGGTGGATT CATCCGAGAAGATGCGCGCCTACTACCGCCAGTGGGCCCCTGTCTATTCGCCGGG CTCCCATCGCCAGTACTCCAACCCCAGCATAGGGCTGTTCGGCCACCTGGCGGCG AGCAGCCTGAAGCAGCCATTTGCCCAGTTGATGGAGCAGACCCTGCTGCCCGGG CTCGGCATGCACCACACCTATGTCAATGTG, AACAGCCTCAGCAGCCGGTTACGGAAAATACGTTATTTGAAGTGGGTTCGCTGAGT AAAACGTTTGCTGCCACCTTGGCGTCCTATGCGCAGGTGAGCGGTAAGCTGTCTTT GGATCAAAGCGTTAGCCATTACGTTCCAGAGTTGCGTGGCAGCAGCTTTGACCAC GTTAGCGTACTCAATGTGGGCACGCATACCTCAGGCCTACAGCTATTTATGCCGGA AGATATTAAAAATACCACACAGCTGATGGCTTATCTAAAAGCATGGAAACCTGCCG ATGCGGCTGGAACCCATCGCGTTTATTCCAATATCGGTACTGGTTTGCTAGGGATG ATTGCGGCGAA and GCCGAGGCTTACGGGATCAAGACCGGCTCGGCGGATCTGCTGAAGTTTACCGAG GCCAACATGGGGTATCAGGGAGATGCCGCGCTAAAAACGCGGATCGCGCTGACC CATACCGGTTTCTACTCGGTGGGAGACATGACTCAGGGGCTGGGTTGGGAGAGCT ACGCCTATCCGTTGACCGAGCAGGCGCTGCTGGCGGGCAACTCCCCGGCGGTGA GCTTCCAGGCCAATCCGGTTACGCGCTTTG. The first DNA control mix may include the following internal control sequence: GAGAGGATGACCAGCCACACTGGAACTGAGACACGGTCCAGACTCCTACGGGAG GCAGCAGTGGGGAATATTGCACAATGGGCG. A DNA control mix may include a combination of the one or more said group of sequences and the said internal control sequence. A DNA control mix may include any combination of sequences from the first control mix, the second control mix and the internal control sequence. [SEQ. ID NOS 273-276]

A second DNA control mix may include one or more sequences selected from the group consisting of: AACTTTCACAGGTGTGCTGGGTGCGGTTTCTGTGGCGAAAAAAGAGATGGCGCTG AATGATCCGGCGGCAAAATACCAGCCGGAGCTGGCTCTGCCGCAGTGGAAGGGG ATCACATTGCTGGATCTGGCTACCTATACCGCAGGCGGACTGCCGTTACAGGTGC CGGATGCGGTAAAAAGCCGTGCGGATCTGCTGAATTTCTATCAGCAGTGGCAGCC GTCCCGGAAACCGGGCGATATGCGTCTGTATGCAAACAGCAGTATCGGCCTGTTT GGTGCTCTGACCGCAAACGCGGCGGGGATGCCGTATGAGCAGTTGCTGACTGCA CGGATCCTGGCACCGCTGGGGTTATCTCACACCTTTATTACTGTGCCGGAAAGTG CGCAAAGCCAGTATGCGTACGG, TCGGTAAAGCCGATGTTGCGGCGAACAAACCCGTCACCCCGCAAACCCTGTTTGA GCTGGGCTCTATAAGTAAAACCTTCACCGGCGTACTGGGCGGCGATGCCATTGCC CGGGGTGAAATAGCGCTGGGCGATCCGGTAGCAAAATACTGGCCTGAGCTCACG GGCAAGCAGTGGCAGGGCATTCGCATGCTGGATCTGGCAACCTATACCGCAGGC GGTCTGCCGTTACAGGTGCCGGATGAGGTCACGGATACCGCCTCTCTGCTGCGCT TTTATCAAAACTGGCAGCCGCAGTGGAAG and CCGAAGCCTATGGCGTGAAATCCAGCGTTATTGATATGGCCCGCTGGGTTCAGGC CAACATGGATGCCAGCCACGTTCAGGAGAAAACGCTCCAGCAGGGCATTGC. The second DNA control mix may include the following internal control sequence: GAGAGGATGACCAGCCACACTGGAACTGAGACACGGTCCAGACTCCTACGGGAG GCAGCAGTGGGGAATATTGCACAATGGGCG. A DNA control mix may include a combination of the one or more said group of sequences and the said internal control sequence. A DNA control mix may include any combination of sequences from the first control mix, the second control mix and the internal control sequence. [SEQ. ID NOS 277-280]

In one example, the kit includes two primer-probe multiplex mix vials including internal controls and two DNA control mix vials. The two primer-probe multiplex mixes may provide for identification of up to six antibiotic resistance genes and internal controls. A first primer-probe mix may include sequences for detecting gene families which are OXA-143-like, OXA-23-like, OXA-51-like and internal controls. A second primer-probe mix may include sequences for detecting gene families which are OXA-48-like, OXA-58-like, OXA-24/40-like and internal controls. A first DNA control mix may include DNA sequences for OXA-143, OXA-23, OXA-51 and an internal control DNA sequence. A second DNA control mix may include DNA sequences for OXA-48, OXA-58 and OXA 24/40 and an internal control DNA sequence.

It is contemplated that the combination of gene families may vary. For example, a primer-probe mix may include sequences for detecting any combination of the following genes: OXA-143-like, OXA-23-like, OXA-51-like, OXA-48-like, OXA-58-like and OXA-24/40-like. It is further contemplated that additional β-lactamase gene targets may be included in the primer-probe mix or mixes.

The first primer-probe mix may include one or more primers and/or probes selected from the group consisting of: AGCACATACAGAATATGTCCCTGC, ACCTGTTAACCAACCTACTTGAGGG, /56-FAM/TTGCAAGACGGACTGGCTTAGACC/3BHQ_1/, CCTGATCGGATTGGAGAACC, CTACCTCTTGAATAGGCGTAACC, /5TEX615/ACGTCGCGCAAGTTCCTGATAGAC/3IAbRQSp/, TAGTGACTGCTAATCCAAATCACAG, GCACGAGCAAGATCATTACCATAGC, /5HEX/AGTTATCCAACAAGGCCAAACTCAACA/3BHQ_1/. [SEQ. ID NOS 119-127] The first primer-probe mix may include one or more internal controls selected from the group consisting of: GAGAGGATGAYCAGCCACAC, CGCCCATTGTSCAATATTCC and 5TYE665/TGAGACACGGTCCAGACTCCTACG/3IAbRQSp. A primer-probe mix may include a combination of the one or more said group of primers and/or probes and the one or more said group of internal controls. The primer-probe mix including internal controls may be a multiplex mix.

The second primer-probe mix may include one or more primers and/or probes selected from the group consisting of: AATCACAGGGCGTAGTTGTG, ACCCACCAGCCAATCTTAGG, /5HEX/TAGCTTGATCGCCCTCGATTTGGG/3BHQ_1/, GTGGGATGGAAAGCCACG, CACTTGCGGGTCTACAGC, /56-FAM/TTACTTTGGGCGAAGCCATGCAAG/3BHQ_1/, CACCTATGGTAATGCTCTTGC, CTGGAACTGCTGACAATGCC, /5TEX615/TGGGAGAAAGATATGACTTTAGGTGAGGCA/3IAbRQSp/. [SEQ. ID NOS 128-136] The second primer-probe mix may include one or more internal controls selected from the group consisting of: GAGAGGATGAYCAGCCACAC, CGCCCATTGTSCAATATTCC and 5TYE665/TGAGACACGGTCCAGACTCCTACG/3IAbRQSp. A primer-probe mix may include a combination of the one or more said group of primers and/or probes and the one or more said group of internal controls. The primer-probe mix including internal controls may be a multiplex mix.

The kit may include exactly two primer and/or probe mixes, a first primer and/or probe mix including one or more primers and/or probes selected from the group consisting of: AGCACATACAGAATATGTCCCTGC, ACCTGTTAACCAACCTACTTGAGGG, /56-FAM/TTGCAAGACGGACTGGCTTAGACC/3BHQ_1/, CCTGATCGGATTGGAGAACC, CTACCTCTTGAATAGGCGTAACC, /5TEX615/ACGTCGCGCAAGTTCCTGATAGAC/3IAbRQSp/, TAGTGACTGCTAATCCAAATCACAG, GCACGAGCAAGATCATTACCATAGC, /5HEX/AGTTATCCAACAAGGCCAAACTCAACA/3BHQ_1/, GAGAGGATGAYCAGCCACAC, CGCCCATTGTSCAATATTCC and 5TYE665/TGAGACACGGTCCAGACTCCTACG/3IAbRQSp; and a second primer and/or probe mix including one or more primers and/or probes selected from the group consisting of: AATCACAGGGCGTAGTTGTG, ACCCACCAGCCAATCTTAGG, /5HEX/TAGCTTGATCGCCCTCGATTTGGG/3BHQ_1/, GTGGGATGGAAAGCCACG, CACTTGCGGGTCTACAGC, /56-FAM/TTACTTTGGGCGAAGCCATGCAAG/3BHQ_1/, CACCTATGGTAATGCTCTTGC, CTGGAACTGCTGACAATGCC, /5TEX615/TGGGAGAAAGATATGACTTTAGGTGAGGCA/3IAbRQSp/, GAGAGGATGAYCAGCCACAC, CGCCCATTGTSCAATATTCC and 5TYE665/TGAGACACGGTCCAGACTCCTACG/3IAbRQSp. Primers and/or probes included in this group may or may not be degenerate at any nucleotide position. [SEQ. ID NOS 216-239]

A first DNA control mix may include one or more sequences selected from the group consisting of: AGCACATACAGAATATGTCCCTGCATCAACATTTAAGATGCTAAATGCCTTAATTGG ACTAGAAAATCATAAAGCTACAACAACTGAGATTTTCAAATGGGACGGTAAAAAGA GATCTTATCCCATGTGGGAAAAAGATATGACTTTAGGTGATGCCATGGCACTTTCA GCAGTTCCTGTATATCAAGAACTTGCAAGACGGACTGGCTTAGACCTAATGCAAAA AGAAGTTAAACGGGTTGGTTTTGGTAATATGAACATTGGAACACAAGTTGATAACTT CTGGTTGGTTGGCCCCCTCAAGATTACACCAATACAAGAGGTTAATTTTGCCGATG ATTTTGCAAATAATCGATTACCCTTTAAATTAGAGACTCAAGAAGAAGTTAAAAAAAT GCTTCTGATTAAAGAATTCAATGGTAGTAAAATTTATGCAAAAAGCGGCTGGGGAA TGGATGTAACCCCTCAAGTAGGTTGGTTAACAGGT, CCTGATCGGATTGGAGAACCAGAAAACGGATATTAATGAAATATTTAAATGGAAGG GCGAGAAAAGGTCATTTACCGCTTGGGAAAAAGACATGACACTAGGAGAAGCCAT GAAGCTTTCTGCAGTCCCAGTCTATCAGGAACTTGCGCGACGTATCGGTCTTGATC TCATGCAAAAAGAAGTAAAACGTATTGGTTTCGGTAATGCTGAAATTGGACAGCAG GTTGATAATTTCTGGTTGGTAGGACCATTAAAGGTTACGCCTATTCAAGAGGTAG and TAGTGACTGCTAATCCAAATCACAGCGCTTCAAAATCTGATGAAAAAGCAGAGAAA ATTAAAAATTTATTTAACGAAGTACACACTACGGGTGTTTTAGTTATCCAACAAGGC CAAACTCAACAAAGCTATGGTAATGATCTTGCTCGTGC. The first DNA control mix may include the following internal control sequence: GAGAGGATGACCAGCCACACTGGAACTGAGACACGGTCCAGACTCCTACGGGAG GCAGCAGTGGGGAATATTGCACAATGGGCG. A DNA control mix may include a combination of the one or more said group of sequences and the said internal control sequence. A DNA control mix may include any combination of sequences from the first control mix, the second control mix and the internal control sequence. [SEQ. ID NOS 281-284]

A second DNA control mix may include one or more sequences selected from the group consisting of: AATCACAGGGCGTAGTTGTGCTCTGGAATGAGAATAAGCAGCAAGGATTTACCAAT AATCTTAAACGGGCGAACCAAGCATTTTTACCCGCATCTACCTTTAAAATTCCCAAT AGCTTGATCGCCCTCGATTTGGGCGTGGTTAAGGATGAACACCAAGTCTTTAAGTG GGATGGACAGACGCGCGATATCGCCACTTGGAATCGCGATCATAATCTAATCACC GCGATGAAATATTCAGTTGTGCCTGTTTATCAAGAATTTGCCCGCCAAATTGGCGA GGCACGTATGAGCAAGATGCTACATGCTTTCGATTATGGTAATGAGGACATTTCGG GCAATGTAGACAGTTTCTGGCTCGACGGTGGTATTCGAATTTCGGCCACGGAGCA AATCAGCTTTTTAAGAAAGCTGTATCACAATAAGTTACACGTATCGGAGCGCAGCC AGCGTATTGTCAAACAAGCCATGCTGACCGAAGCCAATGGTGACTATATTATTCGG GCTAAAACTGGATACTCGACTAGAATCGAACCTAAGATTGGCTGGTGGGT, GTGGGATGGAAAGCCACGTTTTTTTAAAGCATGGGACAAAGATTTTACTTTGGGCG AAGCCATGCAAGCATCTACAGTGCCTGTATATCAAGAATTGGCACGTCGTATTGGT CCAAGCTTAATGCAAAGTGAATTGCAACGTATTGGTTATGGCAATATGCAAATAGG CACGGAAGTTGATCAATTTTGGTTGAAAGGGCCTTTGACAATTACACCTATACAAG AAGTAAAGTTTGTGTATGATTTAGCCCAAGGGCAATTGCCTTTTAAACCTGAAGTTC AGCAACAAGTGAAAGAGATGTTGTATGTAGAGCGCAGAGGGGAGAATCGTCTATA TGCTAAAAGTGGCTGGGGAATGGCTGTAGACCCGCAAGTG, CACTTGCGGGTCTACAGCCATTCCCCAGCCACTTTTAGCATATAGACGATTCTCCC CTCTGCGCTCTACATACAACATCTCTTTCACTTGTTGCTGAACTTCAGGTTTAAAAG GCAATTGCCCTTGGGCTAAATCATACACAAACTTTACTTCTTGTATAGGTGTAATTG TCAAAGGCCCTTTCAACCAAAATTGATCAACTTCCGTGCCTATTTGCATATTGCCAT AACCAATACGTTGCAATTCACTTTGCATTAAGCTTGGACCAATACGACGTGCCAATT CTTGATATACAGGCACTGTAGATGCTTGCATGGCTTCGCCCAAAGTAAAATCTTTGT CCCATGCTTTAAAAAAACGTGGCTTTCCATCCCAC, and CACCTATGGTAATGCTCTTGCACGAGCAAATAAAGAATATGTCCCTGCATCAACATT TAAGATGCTAAATGCTTTAATCGGGCTAGAAAATCATAAAGCAACAACAAATGAGAT TTTCAAATGGGATGGTAAAAAAAGAACTTATCCTATGTGGGAGAAAGATATGACTTT AGGTGAGGCAATGGCATTGTCAGCAGTTCCAG. The second DNA control mix may include the following internal control sequence: GAGAGGATGACCAGCCACACTGGAACTGAGACACGGTCCAGACTCCTACGGGAG GCAGCAGTGGGGAATATTGCACAATGGGCG. A DNA control mix may include a combination of the one or more said group of sequences and the said internal control sequence additional β-lactamase. A DNA control mix may include any combination of sequences from the first control mix, the second control mix and the internal control sequence. [SEQ. ID NOS 285-289]

In one example, the kit includes one primer-probe multiplex mix vials including internal control and one DNA control mix vial. A primer-probe mix may include sequences for detecting MCR gene families and internal control.

The primer-probe mix may include primers and/or probes selected from the group consisting of: CCGTGTATGTTCAGCTAT, CTTATCCATCACGCCTTT, /5TEX615/TATGATGTCGATACCGCCAAATACCA/3IAbRQSp/, CTGTATGTCAGCGATCAT, GATGCCAGTTTGCTTATCC, /56-FAM/AAGTCTGGG/ZEN/TGAGAACGGTGTCTAT/3IABkFQ/, CAGTCAGTATGCGAGTTTC, AAAATTCGCCAAGCCATC, and /5HEX/TGCATAAGC/ZEN/CAGTGCGTTTTTATAT/3IABkFQ/. The primer-probe mix may include one or more internal controls selected from the group consisting of: GAGAGGATGAYCAGCCACAC, CGCCCATTGTSCAATATTCC and 5TYE665/TGAGACACGGTCCAGACTCCTACG/3IAbRQSp. The primer-probe mix may include a combination of the one or more said group of primers and/or probes and the one or more said group of internal controls. The primer-probe mix including internal controls may be a multiplex mix. [SEQ. ID NOS 137-145]

A DNA control mix may include one or more sequences selected from the group consisting of

ATGATGCAGCATACTTCTGTGTGGTACCGACGCTCGGTCAGTCCGTTTGT
TCTTGTGGGAGTGTTGCCGTTTTCTTGACCGCGACCGCCAATCTTACCTT
TTTTGATAAAATCAGCCAAACCTATCCCATCGCGGACAATCTCGGCTTTG
TGCTGACGATCGCTGTCGTGCTCTTTGGCGCGATGCTACTGATCACCACG
CTGTTATCATCGTATCGCTATGTGCTAAAGCCTGTGTTGATTTTGCTATT
AATCATGGGCGCGGTGACCAGTTATTTTACTGACACTTATGGCACGGTCT
ATGATACGACCATGCTCCAAAATGCCCTACAGACCGACCAAGCCGAGACC
AAGGATCTATTAAACGCAGCGTTTATCATGCGTATCATTGGTTTGGGTGT
GCTACCAAGTTTGCTTGTGGCTTTTGTTAAGGTGGATTATCCGACTTGGG
GCAAGGGTTTGATGCGCCGATTGGGCTTGATCGTGGCAAGTCTTGCGCTG
ATTTTACTGCCTGTGGTGGCGTTCAGCAGTCATTATGCCAGTTTCTTTCG
CGTGCATAAGCCGCTGCGTAGCTATGTCAATCCGATCATGCCAATCTACT
CGGTGGGTAAGCTTGCCAGTATTGAGTATAAAAAAGCCAGTGCGCCAAAA
GATACCATTTATCACGCCAAAGACGCGGTACAAGCAACCAAGCCTGATAT
GCGTAAGCCACGCCTAGTGGTGTTCGTCGTCGGTGAGACGGCACGCGCCG
ATCATGTCAGCTTCAATGGCTATGAGCGCGATACTTTCCCACAGCTTGCC
AAGATCGATGGCGTGACCAATTTTAGCAATGTCACATCGTGCGGCACATC
GACGGCGTATTCTGTGCCGTGTATGTTCAGCTATCTGGGCGCGGATGAGT
ATGATGTCGATACCGCCAAATACCAAGAAAATGTGCTGGATACGCTGGAT
CGCTTGGGCGTAAGTATCTTGTGGCGTGATAATAATTCGGACTCAAAAGG
CGTGATGGATAAGCTGCCAAAAGCGCAATTTGCCGATTATAAATCCGCGA
CCAACAACGCCATCTGCAACACCAATCCTTATAACGAATGCCGCGATGTC
GGTATGCTCGTTGGCTTAGATGACTTTGTCGCTGCCAATAACGGCAAAGA
TATGCTGATCATGCTGCACCAAATGGGCAATCACGGGCCTGCGTATTTTA
AGCGATATGATGAAAAGTTTGCCAAATTCACGCCAGTGTGTGAAGGTAAT
GAGCTTGCCAAGTGCGAACATCAGTCCTTGATCAATGCTTATGACAATGC
CTTGCTTGCCACCGATGATTTCATCGCTCAAAGTATCCAGTGGCTGCAGA
CGCACAGCAATGCCTATGATGTCTCAATGCTGTATGTCAGCGATCATGGC
GAAAGTCTGGGTGAGAACGGTGTCTATCTACATGGTATGCCAAATGCCTT
TGCACCAAAAGAACAGCGCAGTGTGCCTGCATTTTTCTGGACGGATAAGC
AAACTGGCATCACGCCAATGGCAACCGATACCGTCCTGACCCATGACGCG
ATCACGCCGACATTATTAAAGCTGTTTGATGTCACCGCGGACAAAGTCAA
AGACCGCACCGCATTCATCCGCTGA
and
ATGACATCACATCACTCTTGGTATCGCTATTCTATCAATCCTTTTGTGCT
GATGGGTTTGGTGGCGTTATTTTTGGCAGCGACAGCGAACCTGACATTTT
TTGAAAAAGCGATGGCGGTCTATCCTGTATCGGATAACTTAGGCTTTATC
ATCTCAATGGCGGTGGCGGTGATGGGTGCTATGCTACTGATTGTCGTGCT
GTTATCCTATCGCTATGTGCTAAAGCCTGTCCTGATTTTGCTACTGATTA
TGGGTGCGGTGACGAGCTATTTTACCGATACTTATGGCACGGTCTATGAC
ACCACCATGCTCCAAAATGCCATGCAAACCGACCAAGCCGAGTCTAAGGA
CTTGATGAATTTGGCGTTTTTTGTGCGAATTATCGGGCTTGGCGTGTTGC
CAAGTGTGTTGGTCGCAGTTGCCAAAGTCAATTATCCAACATGGGGCAAA
GGTCTGATTCAGCGTGCGATGACATGGGGTGTCAGCCTTGTGCTGTTGCT
TGTGCCGATTGGACTATTTAGCAGTCAGTATGCGAGTTTCTTTCGGGTGC
ATAAGCCAGTGCGTTTTTATATCAACCCGATTACGCCGATTTATTCGGTG
GGTAAGCTTGCCAGTATCGAGTACAAAAAAGCCACTGCGCCAACAGACAC
CATCTATCATGCCAAAGACGCCGTGCAGACCACCAAGCCGAGCGAGCGTA
AGCCACGCCTAGTGGTGTTCGTCGTCGGTGAGACGGCGCGTGCTGACCAT
GTGCAGTTCAATGGCTATGGCCGTGAGACTTTCCCGCAGCTTGCCAAAGT
TGATGGCTTGGCGAATTTTAGCCAAGTGACATCGTGTGGCACATCGACGG
CGTATTCTGTGCCGTGTATGTTCAGCTATTTGGGTCAAGATGACTATGAT
GTCGATACCGCCAAATACCAAGAAAATGTGCTAGATACGCTTGACCGCTT
GGGTGTGGGTATCTTGTGGCGTGATAATAATTCAGACTCAAAAGGCGTGA
TGGATAAGCTACCTGCCACGCAGTATTTTGATTATAAATCAGCAACCAAC
AATACCATCTGTAACACCAATCCCTATAACGAATGCCGTGATGTCGGTAT
GCTTGTCGGGCTAGATGACTATGTCAGCGCCAATAATGGCAAAGATATGC
TCATCATGCTACACCAAATGGGCAATCATGGGCCGGCGTACTTTAAGCGT
TATGATGAGCAATTTGCCAAATTCACCCCCGTGTGCGAAGGCAACGAGCT
TGCCAAATGCGAACACCAATCACTCATCAATGCCTATGACAATGCGCTAC
TTGCGACTGATGATTTTATCGCCAAAAGCATCGATTGGCTAAAAACGCAT
GAAGCGAACTACGATGTCGCCATGCTCTATGTCAGTGACCACGGCGAGAG
CTTGGGCGAAAATGGTGTCTATCTGCATGGTATGCCAAATGCCTTTGCAC
CAAAAGAACAGCGAGCTGTGCCTGCGTTTTTTTGGTCAAATAATACGACA
TTCAAGCCAACTGCCAGCGATACTGTGCTGACGCATGATGCGATTACGCC
AACACTGCTTAAGCTGTTTGATGTCACAGCGGGCAAGGTCAAAGACCGCG
CGGCATTTATCCAGTAA.

The DNA control mix may include the following internal control sequence: GAGAGGATGACCAGCCACACTGGAACTGAGACACGGTCCAGACTCCTACGGGAG GCAGCAGTGGGGAATATTGCACAATGGGCG. A DNA control mix may include a combination of the one or more said group of sequences and the said internal control sequence. [SEQ. ID NOS 290-292]

In one example, the kit includes one primer-probe multiplex mix vial including internal control and one DNA control mix vial. A primer-probe mix may include sequences for detecting TEM-like and SHV-like gene families and internal control.

The primer-probe mix may include primers and/or probes selected from the group consisting of: AGATCAGTTGGGTGCACG, TGCTTAATCAGTGAGGCACC, /56-FAM/ATGAAGCCA/ZEN/TACCAAACGACGAGC/3IABkFQ/, CTGGAGCGAAAGATCCACTA, ATCGTCCACCATCCACTG, and /5HEX/CCAGATCGG/ZEN/CGACAACGTCACC/3IABkFQ/. The primer-probe mix may include one or more internal controls selected from the group consisting of: GAGAGGATGAYCAGCCACAC, CGCCCATTGTSCAATATTCC and 5TYE665/TGAGACACGGTCCAGACTCCTACG/3IAbRQSp. The primer-probe mix may include a combination of the one or more said group of primers and/or probes and the one or more said group of internal controls. The primer-probe mix including internal controls may be a multiplex mix. [SEQ. ID NOS 240-248]

A DNA control mix may include one or more sequences selected from the group consisting of: AGATCAGTTGGGTGCACGAGTGGGTTACATCGAACTGGATCTCAACAGCGGTAAG ATCCTTGAGAGTTTTCGCCCCGAAGAACGTTTTCCAATGATGAGCACTTTTAAAGTT CTGCTATGTGGTGCGGTATTATCCCGTGTTGACGCCGGGCAAGAGCAACTCGGTC GCCGCATACACTATTCTCAGAATGACTTGGTTGAGTACTCACCAGTCACAGAAAAG CATCTTACGGATGGCATGACAGTAAGAGAATTATGCAGTGCTGCCATAACCATGAG TGATAACACTGCGGCCAACTTACTTCTGACAACGATCGGAGGACCGAAGGAGCTA ACCGCTTTTTTGCACAACATGGGGGATCATGTAACTCGCCTTGATCGTTGGGAACC GGAGCTGAATGAAGCCATACCAAACGACGAGCGTGACACCACGACGCCTGCAGC AATGGCAACAACGTTGCGCAAACTATTAACTGGCGAACTACTTACTCTAGCTTCCC GGCAACAATTAATAGACTGGATGGAGGCGGATAAAGTTGCAGGACCACTTCTGCG CTCGGCCCTTCCGGCTGGCTGGTTTATTGCTGATAAATCTGGAGCCAGTGAGCGT GGGTCTCGCGGTATCATTGCAGCACTGGGGCCAGATGGTAAGCCCTCCCGTATCG TAGTTATCTACACGACGGGGAGTCAGGCAACTATGGATGAACGAAATAGACAGATC GCTGAGATAGGTGCCTCACTGATTAAGCA and CTGGAGCGAAAGATCCACTATCGCCAGCAGGATCTGGTGGACTACTCGCCGGTCA GCGAAAAACACCTTGCCGACGGCATGACGGTCGGCGAACTCTGCGCCGCCGCCA TTACCATGAGCGATAACAGCGCCGCCAATCTGCTGCTGGCCACCGTCGGCGGCC CCGCAGGATTGACTGCCTTTTTGCGCCAGATCGGCGACAACGTCACCCGCCTTGA CCGCTGGGAAACGGAACTGAATGAGGCGCTTCCCGGCGACGCCCGCGACACCAC TACCCCGGCCAGCATGGCCGCGACCCTGCGCAAGCTGCTGACCAGCCAGCGTCT GAGCGCCCGTTCGCAACGGCAGCTGCTGCAGTGGATGGTGGACGAT. The DNA control mix may include the following internal control sequence: GAGAGGATGACCAGCCACACTGGAACTGAGACACGGTCCAGACTCCTACGGGAG GCAGCAGTGGGGAATATTGCACAATGGGCG. A DNA control mix may include a combination of the one or more said group of sequences and the said internal control sequence. [SEQ. ID NOS 293-295]

The primer-probe multiplex mix may comprise different oligonucleotide sequences. An oligonucleotide sequence may be utilized as a primer. An oligonucleotide sequence may be utilized as a probe. An oligonucleotide sequence may be utilized as an internal control sequence. The oligonucleotide concentration of a primer and/or probe sequence may range from 0.05 μM to 60 μM. For example, the oligonucleotide concentration of a primer and/or probe sequence may range from 3 μM to 8 μM. For example, the oligonucleotide concentration of an internal control sequence may range from 2 μM to 6 μM. For example, the oligonucleotide concentration of an internal control sequence may range from 2 μM to 8 μM. The vial oligonucleotide concentrations may be prepared as a 10× stock solution.

The synthetic gene size of a DNA control sequence may be from about 84 bp to about 533 bp. The concentration of a DNA control sequence may be about 25 ng/μl. The concentration of a DNA control sequence may be from 0.033 ng/μL to about 0.5 ng/μl.

The present teachings provide methods for detection of β-lactamase gene families from a biological sample. Preferably, the sample includes Gram-negative bacteria. The method may include sample processing. The method may include extracting DNA from the sample. The method may include extracting RNA from the sample. The method may include the use of assays of the present teachings. The assays may be included in a kit or kits. The method may include employing the kit of the present teachings for the detection of multiple β-lactamase gene families from a biological sample.

The method may include employing the kit for analysis of nucleic acid contained in a clinical sample. The method may include employing the kit for analysis of DNA extracted from a clinical sample. The method may include employing the kit for analysis of DNA extracted from an overnight bacterial culture of a clinical sample.

The method may include amplifying a targeted DNA sequence by real-time polymerase reaction. The method may include amplifying several targeted DNA sequences by multiplex real-time polymerase reaction. The method may include analyzing the amplified sequences or amplicons. The method may include detecting the presence or absence of β-lactamase genes. The method may include detecting the presence or absence of ampC β-lactamase genes. The method may include identifying up to six β-lactamase gene families. The method may include identifying up to nine β-lactamase gene families. The method may include identifying up to fifteen β-lactamase gene families. The method may include identifying up to twenty β-lactamase gene families. The method may include identifying from about six to about thirty β-lactamase gene families. The method may include analyzing collected data.

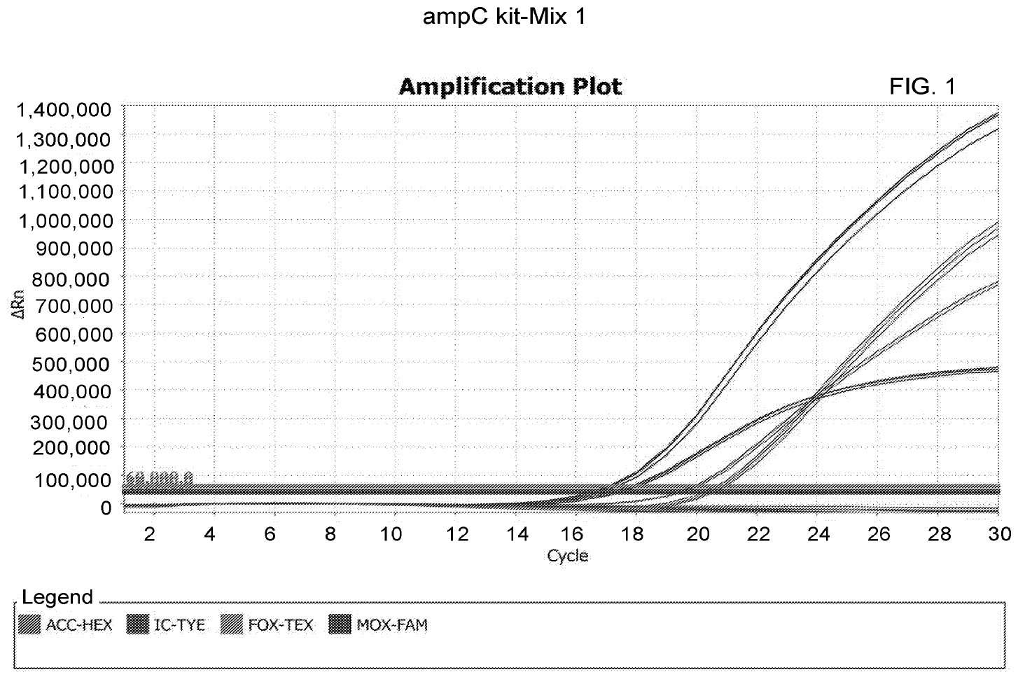

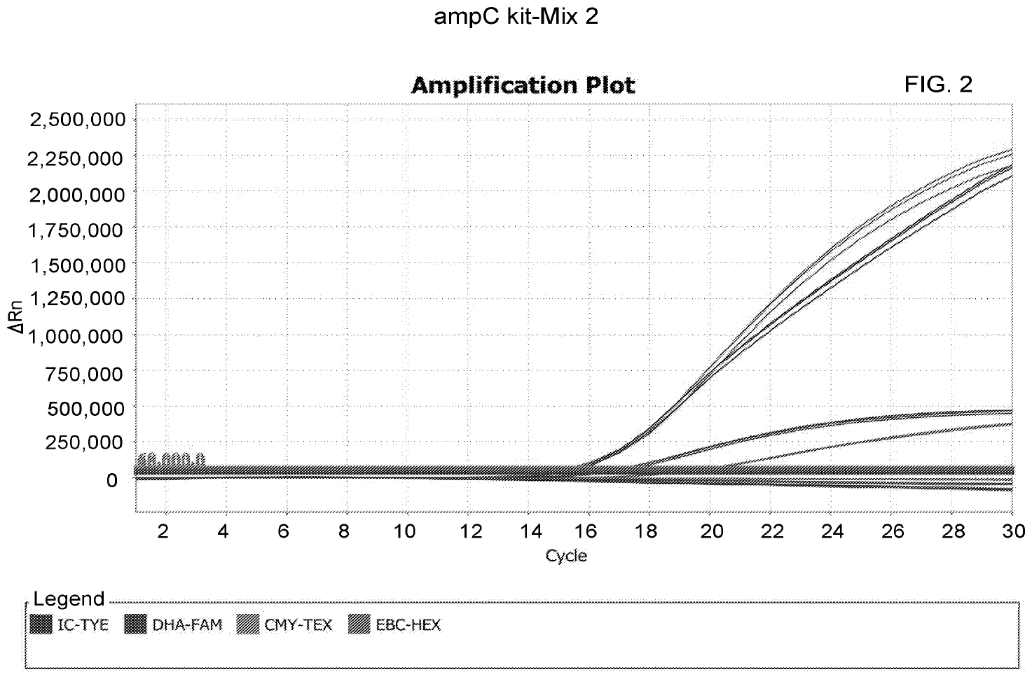

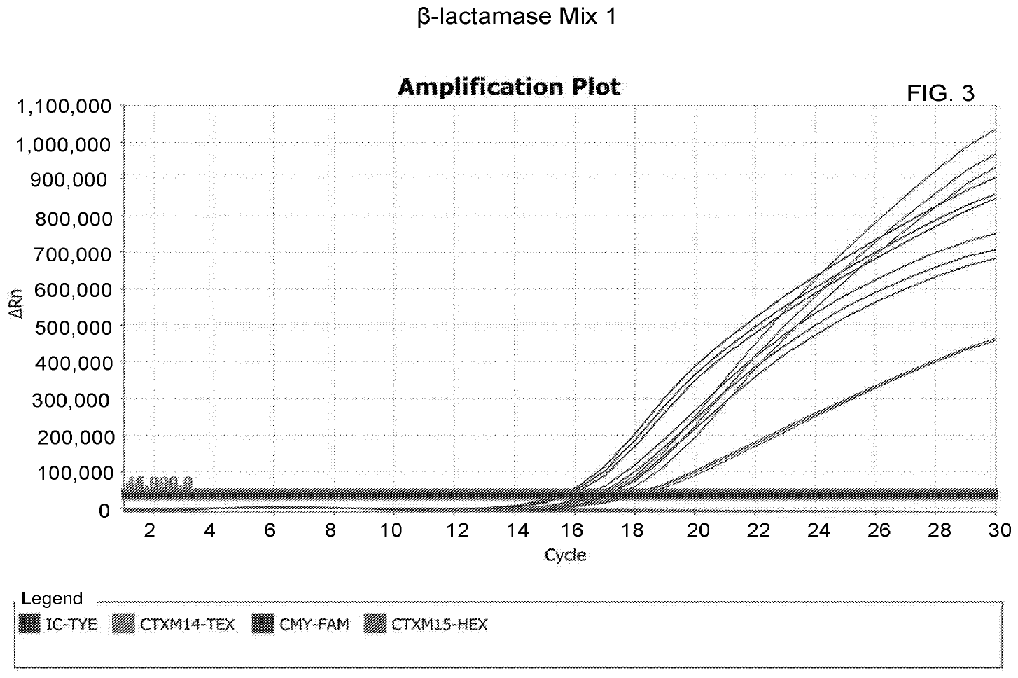

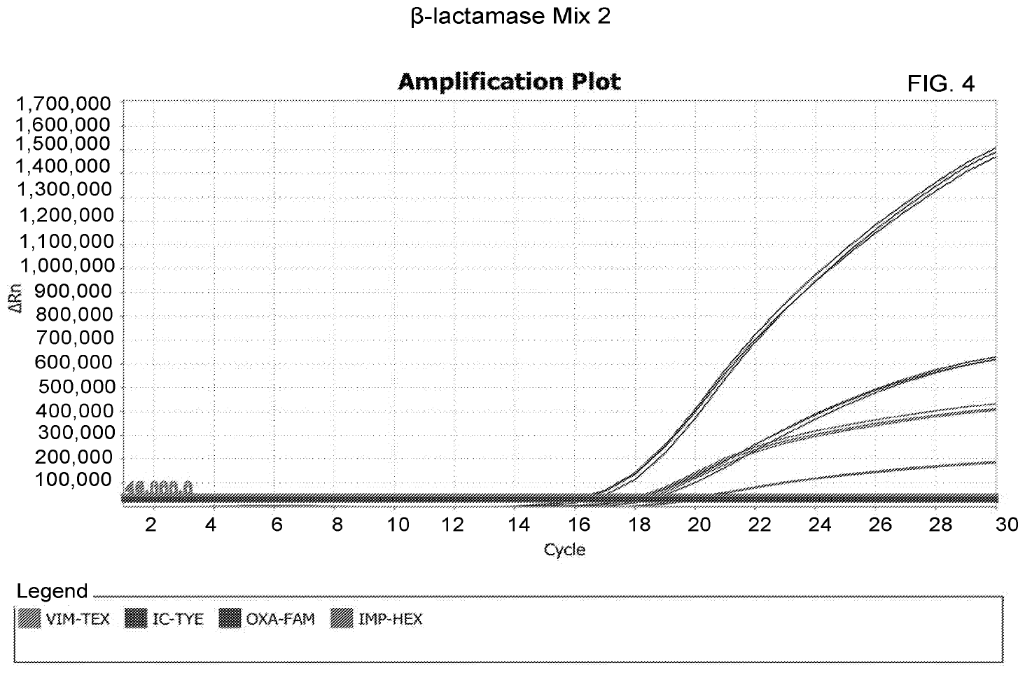

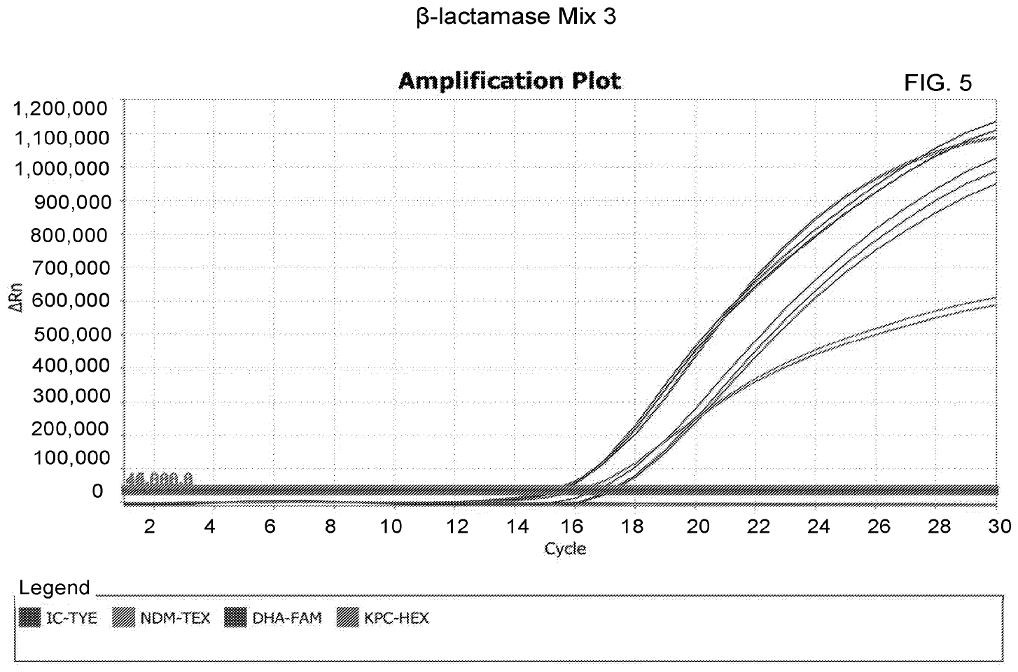

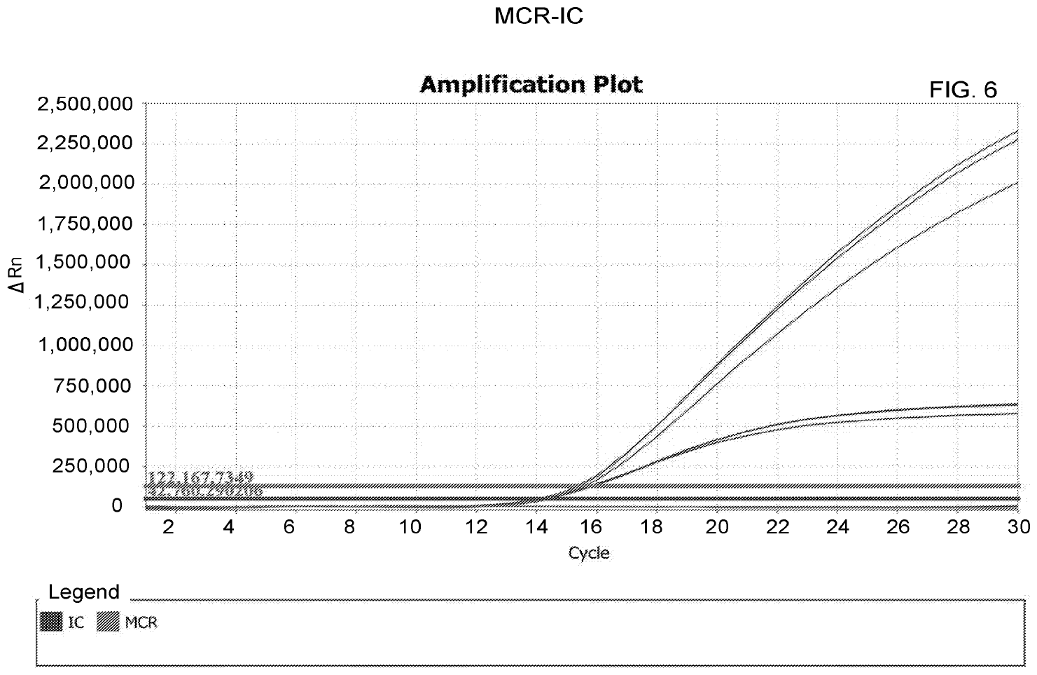

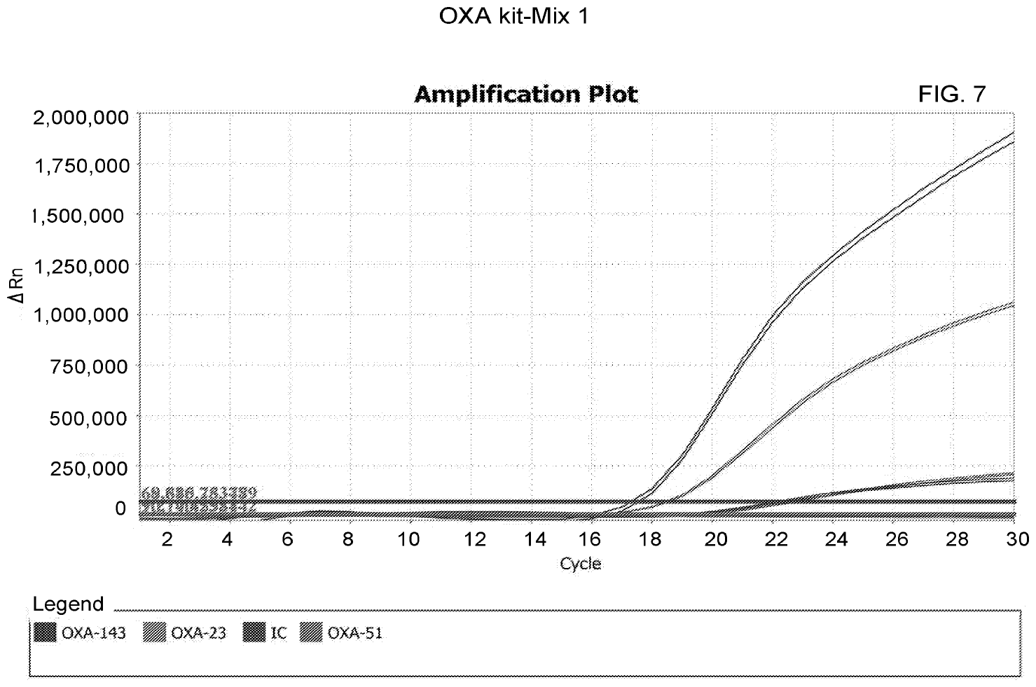

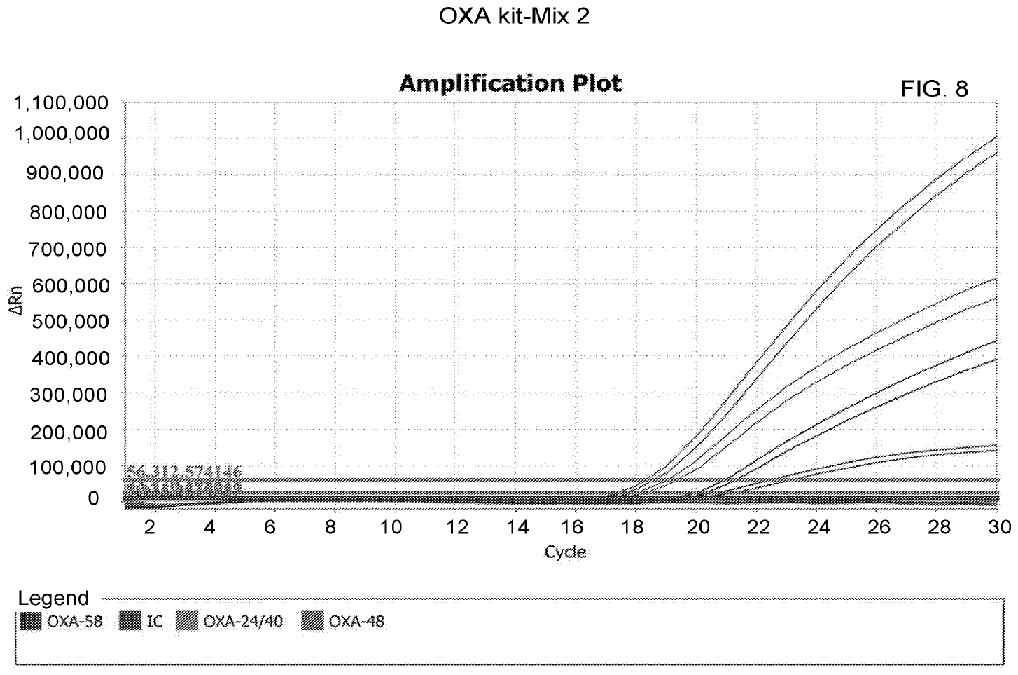

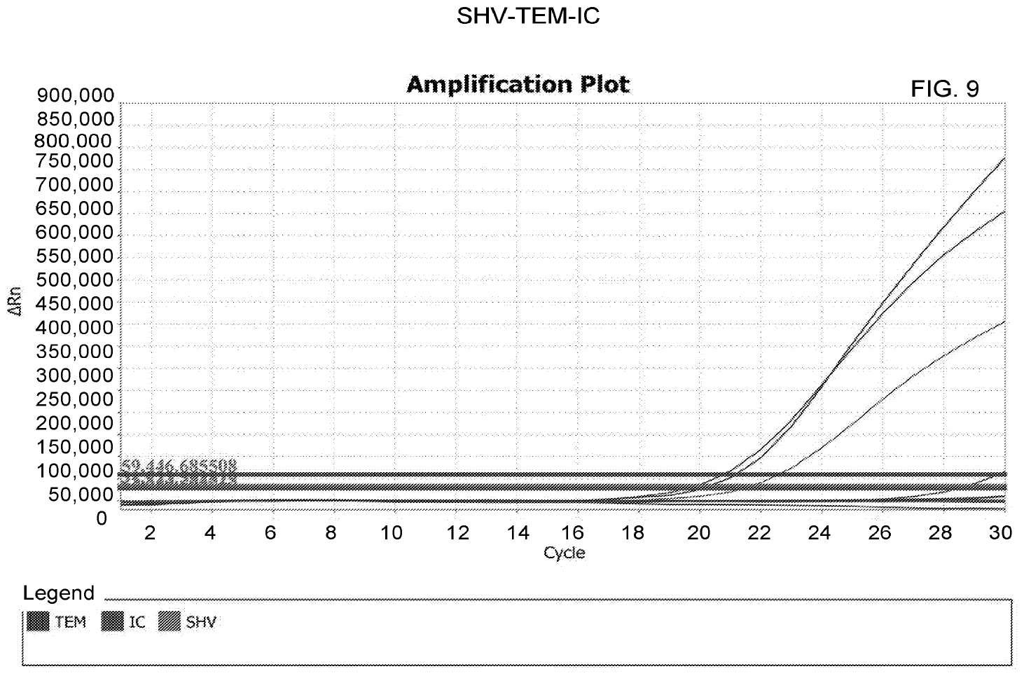

Examples of real-time PCR amplification curves obtained on the ABI QS7 Flex-Real-Time System for some of the multiplex mixes described herein are shown in FIGS. 1-9. FIG. 1 depicts an amplification plot of an exemplary mix 1 including ampC gene targets. FIG. 2 depicts an amplification plot of an exemplary mix 2 including ampC gene targets. FIG. 3 depicts an amplification plot of an exemplary mix 1 including β-lactamase gene targets. FIG. 4 depicts an amplification plot of an exemplary mix 2 including β-lactamase gene targets. FIG. 5 depicts an amplification plot of an exemplary mix 3 including β-lactamase gene targets. FIG. 6 depicts an amplification plot of an exemplary internal control mix including MCR gene targets. FIG. 7 depicts an amplification plot of an exemplary mix 1 including OXA gene targets. FIG. 8 depicts an amplification plot of an exemplary mix 2 including OXA gene targets. FIG. 9 depicts an amplification plot of an exemplary internal control mix including SHV-TEM gene targets.

The method may include using one or more oligonucleotide primers that are complementary to at least a portion of the nucleic acid sequence of interest. The method may include annealing several pairs of primers to different target DNA sequences. The method may include annealing primer/probe sequences to bacterial nucleic acid sequences comprising targeted antibiotic resistant gene family variants of β-lactamases. The primer and/or probe sequences may anneal with 100% specificity to the target gene variants. The primer and/or probe sequences may anneal with about 95% specificity to the target gene variants. The primer and/or probe sequences may anneal with about 90% to about 100% specificity to the target gene variants. The primer and/or probe sequences may anneal with about 80% to about 100% specificity to the target gene variants.

The method may include using temperature mediated DNA polymerase. The method may include using fluorescent dyes. The method may include the using sequence specific DNA probes including oligonucleotides labeled with a reporter. The method may include using a microarray.

The method may include using a thermal cycler. For example, the kit of the present teachings may be utilized with the following PCR systems: Streck ZULU RT™ PCR System, Applied Biosystems (ABI) QuantStudio 7 (QS7) Flex Real-Time System, ABI 7500 Real-Time PCR System, QIAGEN Rotor-Gene® Q, and CFX96 Touch™ Real-Time PCR Detection System, Applied Biosystems™ 7500 Fast Dx Real-Time PCR Instrument, Roche LightCycler® 480 I and II, and Cepheid SmartCycler®. It is contemplated that any detection system capable of detecting the multiplex fluorescent signal provided in the kit of the present teachings may be suitable.

The method may include real-time monitoring of qPCR reaction products. The probes may generate a signal when hyrodolyzed by the DNA polymerase causing liberation of a detectable fluorescent signal. The real-time monitoring method may employ fluorescence at different wavelengths. The method may include the use of DNA-intercalating fluorescent dyes. The method may include the use of a target specific nucleotide probe labeled with a fluorescent tag at one end. The other end of the hybridization probe may be labeled with a fluorescent quencher. Fluorescent hybridization probes generate a fluorescence signal only when they bind to their target and enable real-time of monitoring of nucleic acid amplification assays.

Surprisingly, some DNA targets detected with these kits, allow for amplification of regions of DNA much larger than the conventional wisdom within the real-time PCR field. For example, most amplicons would traditionally be between 50 to 150 base pairs in size. The present teachings allow for successfully amplified amplicons up to 553 base pairs by real-time PCR.

There may be one or more benefits to detecting larger amplicons. Larger amplicons may, in some cases, provide greater specificity for a specific antibiotic resistance gene family. Detection of larger amplicons may permit detection of an increased number of gene variants within a given resistance gene family. Detection of larger amplicons may also allow confirmation by agarose gel electrophoresis since the molecular sizes of each gene that is detected can be resolved from one another.

The efficiency of detection for each target in a dilution series may be measured for amplicons between 25 base pairs and 2000 base pairs. The efficiency of the PCR for amplicons within this size range may be from 80% to 110%. More specifically, the efficiency of the reactions may be from 90% to 105%. The coefficient of determination may be from 0.98 to 1.1. More specifically, the coefficient of determination may be from 0.99 to 1.0. The limit of detection may be from 0.1 copies to 1×10¹⁰ copies.

Alternate sequences for primer, probes, and DNA controls for β-lactamase gene targets of the present teachings are depicted in Table 2 and Table 3. [SEQ. ID NOS 1-48 and SEQ. ID NOS 49-66]

Primers and/or probes may be degenerate at any nucleotide position. Primers and/or probes may not be degenerate at any nucleotide position. Any suitable fluorophore and/or quencher and nucleic acid sequence combination may be used. For example, a probe may be labeled with a fluorescent tag at one end and a fluorescent quencher at the other end. For example, a probe may be labeled with a fluorescent tag at one end and a fluorescent quencher at the other end. For example, two fluorescent quenchers may be included at one end or within the probe sequence. It is contemplated that the probe sequences of the present teachings may be labeled with any suitable fluorophore and quencher combinations. For example, any fluorophore of the present teachings may be attached to any probe DNA sequence of the present teachings.

TABLE 2

| | Primer/ | |
|---|---|---|
| | Probe | Sequence |
| SEQ ID NO. 1 | MOX F′ | AGA CCC TGT TCG AGA TAG |
| SEQ ID NO. 2 | MOX R′ | ATG GTG ATG CTG TCA AAG |
| SEQ ID NO. 3 | MOX-FAM | 5′-56-FAM-CGT GAG CAA GAC CCT GAC TG-3′BHQ1 |
| SEQ ID NO. 4 | FOX F′ | ACT ATT TCA ACT ATG GGG TT |
| SEQ ID NO. 5 | FOX R′ | TTG TCA TCC AGC TCA AAG |
| SEQ ID NO. 6 | FOX-TEX | 5′-Tex615-TGA CCG CAG CAT AGG CAC-3′BHQ_2 |
| SEQ ID NO. 7 | EBC F′ | GTG GCG GTG ATT TAT GAG |
| SEQ ID NO. 8 | EBC R′ | CGG TGA AGG TTT TAC TTA TAG AA |
| SEQ ID NO. 9 | EBC-HEX | 5′-5HEX/CAGCCGCAC/ZEN/TACTTCACCT/-3′BHQ_1 |
| SEQ ID NO. 10 | DHA F′ | TGCGTACGGTTATGAGAACAA |
| SEQ ID NO. 11 | DHA R′ | CCCAGCGCAGCATATCTT |
| SEQ ID NO. 12 | DHA-FAM | ATGCGGAATCTTACGGCGTGGAAT |
| SEQ ID NO. 13 | CMY F′ | TCC AGC GTT ATT GAT ATG G |
| SEQ ID NO. 14 | CMY R′ | CAT CTC CCA GCC TAA TCC |
| SEQ ID NO. 15 | CMY-TEX | 5′TexRd-XN/ACATATCGCCAATACGCCAGT/3IAPRQSp/-3′ |
| SEQ ID NO. 16 | ACC F′ | GCCGCTGATGCAGAAGAATA |
| SEQ ID NO. 17 | ACC R′ | TTT GCC GCT AAC CCA TAG TT |
| SEQ ID NO. 18 | ACC-HEX | 5′-/5HEX/TCA CTG CGA/ZEN/CCG ACA TAC CG/3IABkFQ/-3′ |
| SEQ ID NO. 19 | IC F′ | GAG AGG ATG ACC AGC CAC AC |
| SEQ ID NO. 20 | IC R′ | AGT ACT TTA CAA CCC GAA GGC |
| SEQ ID NO. 21 | IC-TYE | 5′/5TYE665/TGA GAC ACG GTC CAG ACT CCT ACG G/3BHQ_2/-3′ |
| SEQ ID NO. 22 | CTX-M-14 | 5′-TTGGTGACGTGGCTCAAA-3′ |
| | F′ | |
| SEQ ID NO. 23 | CTX-M-14 | 5′-ATATCATTGGTGGTGCCGTAG-3′ |
| | R′ | |
| SEQ ID NO. 24 | CTX-M-14- | 5′-/56-FAM/CGTGGACTG/ZEN/TGGGTGATAAGACCG/3IABkFQ/-3′ |
| | FAM | |
| SEQ ID NO. 25 | CTX-M-15 | 5′-GTCACGCTGTTGTTAGGAAGT-3′ |
| | F′ | |
| SEQ ID NO. 26 | CTX-M-15 | 5′-TAATCAATGCCACACCCAGTC-3′ |
| | R′ | |
| SEQ ID NO. 27 | CTX-M-15- | 5′-/5TEX615/AACTTGCCGAATTAGAGCGGCAGT/3BHQ_2/-3′ |
| | TEX615 | |
| SEQ ID NO. 28 | OXA48-F′ | 5′-AGCAGCAAGGATTTACCAATAATC-3′ |
| SEQ ID NO. 29 | OXA48-R′ | 5′-CGTCTGTCCATCCCACTTAAA-3′ |
| SEQ ID NO. 30 | OXA48-HEX | 5′-/5HEX/TAGCTTGAT/ZEN/CGCCCTCGATTTGGG/3IABkFQ/-3′ |
| SEQ ID NO. 31 | CMY F′ | 5′-TCCAGCGTTATTGATATGG-3′ |
| SEQ ID NO. 32 | CMY R′ | 5′-CATCTCCCAGCCTAATCC-3′ |
| SEQ ID NO. 33 | CMY-TxR | 5-/5TexRd-XN/ACATATCGCCAATACGCCAGT/3IAbRQSp/-3′ |
| SEQ ID NO. 34 | NDM F′ | 5′-TTTGATCGTCAGGGATGGC-3′ |
| SEQ ID NO. 35 | NDM R′ | 5′-CAGGTTGATCTCCTGCTTGAT-3′ |
| SEQ ID NO. 36 | NDM-HEX | 5-/5HEX/AGACCGCCC/ZEN/AGATCCTCAACTG/3IABkFQ/-3′ |
| SEQ ID NO. 37 | KPC F′ | 5′-CGCTAAACTCGAACAGGACTT-3′ |
| SEQ ID NO. 38 | KPC R′ | 5′-TAACTTACAGTTGCGCCTGAG-3′ |
| SEQ ID NO. 39 | KPC-FAM | 5′-/5TYE665/ATCGGTGTGTACGCGATGGATACC/3BHQ_2/-3′ |
| SEQ ID NO. 40 | VIM F′ | 5′-CATTCGACCGACAACTTAG-3′ |
| SEQ ID NO. 41 | VIM R′ | 5′-CGTGCGTGACAACTCAT-3′ |
| SEQ ID NO. 42 | VIM-TEX | 5′45TEX615/TGTGCTCTATGGTGGTTGTGCGAT/3BHQ_2/-3′ |
| SEQ ID NO. 43 | DHA F′ | 5′-TGCGTACGGTTATGAGAACAA-3′ |
| SEQ ID NO. 44 | DHA R′ | 5′-CCCAGCGCAGCATATCTT-3′ |
| SEQ ID NO. 45 | DHA-FAM | 5′-/56-FAM/ATGCGGAAT/ZEN/CTTACGGCGTGAAAT/3IABkFQ-3′ |
| SEQ ID NO. 46 | IMP F′ | 5′-ACGTAGTGGTTTGGTTACCTG-3′ |
| SEQ ID NO. 47 | IMP R′ | 5′-AAGCTTCTAAATTTGCGTCACC-3′ |
| SEQ ID NO. 48 | IMP- | 5′-/5HEX/TTTGTTAAA/ZEN/CCGGACGGTCTTGGT/3IABkFQ/-3′ |
| | TYE705 | |

TABLE 3

| | DNA | |
|---|---|---|
| | Control | Sequence |
| SEQ ID NO. 49 | MOX | AACCGGGAGAGCGGGGCCAGCGTCAGCGAGCAGACCCTGTTCGAGATAG |
| | | GATCCGTGAGCAAGACCCTGACTGCGACCCTGGGGGCCTATGCGGTGGTC |
| | | AAGGGAGCGATGCAGCTGGATGACAAGGCGAGCCGGCACGCGCCCTGGC |
| | | TCAAGGGATCCGTCTTTGACAGCATCACCATGGGGGAGCTTGCCACCTAC |
| | | AGC |
| SEQ ID NO. 50 | FOX | GGGGATGGCGGTCGCCGTGCTGAAAGATGGCAAGGCCCACTATTTCAACT |
| | | ATGGGGTTGCCAACCGCGAGAGTGGTCAGCGCGTCAGCGAGCAGACCCT |
| | | GTTCGAGATTGGCTCGGTCAGCAAGACCCTGACCGCGACCCTCGGTGCCT |
| | | ATGCTGCGGTCAAGGGGGGCTTTGAGCTGGATGACAAGGTGAGCCAGCA |
| | | CGCCCCCTGGCTCAAAGGTTCCGCCTTTGATGGTGTGACCAT |
| SEQ ID NO. 51 | EBC | GGACCGTTACGCCGCTGATGAAAGCGCAGGCCATTCCGGGTATGGCGGT |
| | | GGCGGTGATTTATGAGGGTCAGCCGCACTACTTCACCTTCGGTAAAGCCG |
| | | ATGTTGCGGCGAACAAACCTGTCACTCCACAAACCTTGTTCGAACTGGGTT |
| | | CTATAAGTAAAACCTTCACCGGCGTACTCGGTGGCGATGCCATTGCTCGCG |
| | | GTGAAATATCGCTGGGCGA |
| SEQ ID NO. 52 | DHA | GACTGCACGGATCCTGGCACCGCTGGGGTTATCTCACACCTTTATTACTGT |
| | | GCCGGAAAGTGCGCAAAGCCAGTATGCGTACGGTTATGAGAACAAAAAA |
| | | CCGGTCCGCGTGTCGCCGGGACAGCTTGATGCGGAATCTTACGGCGTGGA |
| | | ATCCGCCTCAAAAGATATGCTGCGCTGGGCGGAAATGAATATGGAGCCGT |
| | | CACGGGCCGGTAATGCGGAT |
| SEQ ID NO. 53 | CMY | GCCTGTACACGTTTCTCCGGGACAACTTGACGCCGAAGCCTATGGCGTGA |
| | | AATCCAGCGTTATTGATATGGCCCGCTGGGTTCAGGTCAACATGGACGCC |
| | | AGCCGCGTTCAGGAGAAAACGCTCCAGCAGGGCATTGCGCTTGCGCAGTC |
| | | TCGCTACTGGCGTATTGGCGATATGTACCAGGGATTAGGCTGGGAGATGC |
| | | TGAACTGGCCGCTGAAAGCTGATTCGATCATCAACGGTAGCGACAGCAAA |
| | | GTGGCATTGG |
| SEQ ID NO. 54 | ACC | GAGAGCAAAATTAAAGACACCGTTGATGACCTGATCCAGCCGCTGATGCA |
| | | GAAGAATAATATTCCCGGTATGTCGGTCGCAGTGACCGTCAACGGTAAAA |
| | | ACTACATTTATAACTATGGGTTAGCGGCAAAACAGCCTCAGCAGCCGGTT |
| SEQ ID NO. 55 | IC | AGCTTGTTGGTGGGGTAACGGCTCACCAAGGCGACGATCCCTAGCTGGTC |
| | | TGAGAGGATGACCAGCCACACTGGAACTGAGACACGGTCCAGACTCCTAC |
| | | GGGAGGCAGCAGTGGGGAATATTGCACAATGGGCGCAAGCCTGATGCAG |
| | | CCATGCCGCGTGTATGAAGAAGGCCTTCGGGTTGTAAAGTACTTTCAGCG |
| | | GGGAGGAAGGGAGTAAAGTTAATACCTTTGCTCATTGACGTTACCCGCAG |
| | | AAGAAGCACCGGCTAACTCCG |
| SEQ ID NO. 56 | CTX-M- | CGTTTCGTCTGGATCGCACTGAACCTACGCTGAATACCGCCATTCCCGGCG |
| | 14 | ACCCGAGAGACACCACCACGCCGCGGGCGATGGCGCAGACGTTGCGTCA |
| | | GCTTACGCTGGGTCATGCGCTGGGCGAAACCCAGCGGGCGCAGTTGGTG |
| | | ACGTGGCTCAAAGGCAATACGACCGGCGCAGCCAGCATTCGGGCCGGCTT |
| | | ACCGACGTCGTGGACTGTGGGTGATAAGACCGGCAGCGGCGACTACGGC |
| | | ACCACCAATGATATTGCGGTGATCTGGCCGCAGGGTCGTGCGCCGCTGGT |
| | | TCTGGTGACCTATTTTACCCAGC |
| SEQ ID NO. 57 | CTX-M- | CCGTCACGCTGTTGTTAGGAAGTGTGCCGCTGTATGCGCAAACGGCGGAC |
| | 15 | GTACAGCAAAAACTTGCCGAATTAGAGCGGCAGTCGGGAGGCAGACTGG |
| | | GTGTGGCATTGATTAACACAGC |
| SEQ ID NO. 58 | OXA | AATCACAGGGCGTAGTTGTGCTCTGGAATGAGAATAAGCAGCAAGGATTT |
| | | ACCAATAATCTTAAACGGGCGAACCAAGCATTTTTACCCGCATCTACCTTTA |
| | | AAATTCCCAATAGCTTGATCGCCCTCGATTTGGGCGTGGTTAAGGATGAAC |
| | | ACCAAGTCTTTAAGTGGGATGGACAGACGCGCGATATCGCCACTTGGAAT |
| | | CGCGATCATAATCTAATCACCGCGATGAAATATTCAGTTGTGCCTGTTTAT |
| | | CAAGAATTTGCCCGCCAAATTGGCGAGGCACGTATGAGCAAGATGCTACA |
| | | TGCTTTCGATTATGGTAATGAGGACATTTCGGGCAATGTAGACAGTTTCTG |
| | | GCTCGACGGTGGTATTCGAATTTCGGCCACGGAGCAAATCAGCTTTTTAA |
| | | GAAAGCTGTATCACAATAAGTTACACGTATCGGAGCGCAGCCAGCGTATT |
| | | GTCAAACAAGCCATGCTGACCGAAGCCAATGGTGACTAATTATTCGGGCT |
| | | AAAACTGGATACTCGACTAGAATCGAACCTAAGATTGGCTGGCTGGGT |
| SEQ ID NO. 59 | IC | CGGAGTTAGCCGGTGCTTCTTCTGCGGGTAACGTCAATGAGCAAAGGTAT |
| | | TAACTTTACTCCCTTCCTCCCCGCTGAAAGTACTTTACAACCCGAAGGCCTT |
| | | CTTCATACACGCGGCATGGCTGCATCAGGCTTGCGCCCATTGTGCAATATT |
| | | CCCCACTGCTGCCTCCCGTAGGAGTCTGGACCGTGTCTCAGTTCCAGTGTG |
| | | GCTGGTCATCCTCTCAGACCAGCTAGGGATCGTCGCCTTGGTGAGCCGTTA |
| | | CCCCACCAACAAGCT |
| SEQ ID NO. 60 | CMY | GCCTGTACACGTTTCTCCGGGACAACTTGACGCCGAAGCCTATGGCGTGA |
| | | AATCCAGCGTTATTGATATGGCCCGCTGGGTTCAGGTCAACATGGACGCC |
| | | AGCCGCGTTCAGGAGAAAACGCTCCAGCAGGGCATTGCGCTTGCGCAGTC |
| | | TCGCTACTGGCGTATTGGCGATATGTACCAGGGATTAGGCTGGGAGATGC |
| | | TGAACTGGCCGCTGAAAGCTGATTCGATCATCAACGGTAGCGACAGCAAA |
| | | GTGGCATTGG |
| SEQ ID NO. 61 | NDM | GGCGAAAGTCAGGCTGTGTTGCGCCGCAACCATCCCCTCTTGCGGGGCAA |
| | | GCTGGTTCGACAACGCATTGGCATAAGTCGCAATCCCCGCCGCATGCAGC |
| | | GCGTCCATACCGCCCATCTTGTCCTGATGCGCGTGAGTCACCACCGCCAGC |
| | | GCGACCGGCAGGTTGATCTCCTGCTTGATCCAGTTGAGGATCTGGGCGGT |
| | | CTGGTCATCGGTCCAGGCGGTATCGACCACCAGCACGCGGCCGCCATCCC |
| | | TGACGATCAAAC |
| SEQ ID NO. 62 | KPC | GTATCGCCGTCTAGTTCTGCTGTCTTGTCTCTCATGGCCGCTGGCTGGCTTT |
| | | TCTGCCACCGCGCTGACCAACCTCGTCGCGGAACCATTCGCTAAACTCGAA |
| | | CAGGACTTTGGCGGCTCCATCGGTGTGTACGCGATGGATACCGGCTCAGG |
| | | CGCAACTGTAAGTTACCGCGCTGAGGAGCGCTTCCCACTGTGCAGCTCATT |
| | | CAAGG |
| SEQ ID NO. 63 | VIM | CCATTCAGCCAGATCGGCATCGGCCACGTTCCCCGCAGACGTGCGTGACA |
| | | ACTCATGAATCGCACAACCACCATAGAGCACACTCGCAGACGGGACGTAC |
| | | ACAACTAAGTTGTCGGTCGAATGCGCAGCACCAGGATAGAAGAGTTCTAC |
| | | TGGACCGAAGCGCACTGCGTCCCCGCTCGAGTCCTTCTAGAGAGTGCGTG |
| | | GGAATCTCGTTCCCCTCTACCTCGGCTAGCCGGCGTGTCGACGGTGATGC |
| | | GTACGTTGCCACCCCAGCCGCCCGAAGGACATCAACGCCGCC |
| SEQ ID NO. 64 | DHA | GACTGCACGGATCCTGGCACCGCTGGGGTTATCTCACACCTTTATTACTGT |
| | | GCCGGAAAGTGCGCAAAGCCAGTATGCGTACGGTTATGAGAACAAAAAA |
| | | CCGGTCCGCGTGTCGCCGGGACAGCTTGATGCGGAATCTTACGGCGTGAA |
| | | ATCCGCCTCAAAAGATATGCTGCGCTGGGCGGAAATGAATATGGAGCCGT |
| | | CACGGGCCGGTAATGCGGAT |
| SEQ ID NO. 65 | IC | CGGAGTTAGCCGGTGCTTCTTCTGCGGGTAACGTCAATGAGCAAAGGTAT |
| | | TAACTTTACTCCCTTCCTCCCCGCTGAAAGTACTTTACAACCCGAAGGCCTT |
| | | CTTCATACACGCGGCATGGCTGCATCAGGCTTGCGCCCATTGTGCAATATT |
| | | CCCCACTGCTGCCTCCCGTAGGAGTCTGGACCGTGTCTCAGTTCCAGTGTG |
| | | GCTGGTCATCCTCTCAGACCAGCTAGGGATCGTCGCCTTGGTGAGCCGTTA |
| | | CCCCACCAACAAGCT |
| SEQ ID NO. 66 | IMP | GCGGAGTTAGTTATTGGCTAGTTAAAAATAAAATTGAAGTTTTTTATCCCG |
| | | GCCCGGGGCACACTCAAGATAACGTAGTGGTTTGGTTACCTGAAAAGAAA |
| | | ATTTTATTCGGTGGTTGTTTTGTTAAACCGGACGGTCTTGGTAATTTGGGT |
| | | GACGCAAATTTAGAAGCTTGGCC |

The sequence listing including SEQ ID NOS 1-295 is hereby incorporated by reference for all purposes.

Unless otherwise stated, any numerical values recited herein include all values from the lower value to the upper value in increments of one unit provided that there is a separation of at least 2 units between any lower value and any higher value. As an example, if it is stated that the amount of a component, a property, or a value of a process variable such as, for example, temperature, pressure, time and the like is, for example, from 1 to 90, preferably from 20 to 80, more preferably from 30 to 70, it is intended that intermediate range values such as (for example, 15 to 85, 22 to 68, 43 to 51, 30 to 32 etc.) are within the teachings of this specification. Likewise, individual intermediate values are also within the present teachings. For values which are less than one, one unit is considered to be 0.0001, 0.001, 0.01, or 0.1 as appropriate. These are only examples of what is specifically intended and all possible combinations of numerical values between the lowest value and the highest value enumerated are to be considered to be expressly stated in this application in a similar manner. As can be seen, the teaching of amounts expressed as “parts by weight” herein also contemplates the same ranges expressed in terms of percent by weight. Thus, an expression in the of a range in terms of “at least ‘x’ parts by weight of the resulting composition” also contemplates a teaching of ranges of same recited amount of “x” in percent by weight of the resulting composition.”

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The disclosures of all articles and references, including patent applications and publications, are incorporated by reference for ail purposes. The term “consisting essentially of to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist of, or consist essentially of the elements, ingredients, components or steps.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps.

It is understood that the above description is intended to be illustrative and not restrictive. Many embodiments as well as many applications besides the examples provided will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventors did not consider such subject matter to be part of the disclosed inventive subject matter.

1. A kit for identification of one or more β-lactamase genes, wherein the one or more β-lactamase genes are selected from the group consisting of: CMY, CTX-M, OXA, IMP, VIM, DHA, KPC, and NDM genes, the kit comprising probes comprising the following sequences: ACGCTAACTCCAGCATTGGTCTGT (SEQ ID NO: 154), AACTTGCCGAATTAGAGCRGCAGT (SEQ ID NO: 157), TATCATTGGTGGTGCCGTAGTCGC (SEQ ID NO: 160), TGAGACACGGTCCAGACTCCTACG (SEQ ID NO: 163), TAGCTTGATCGCCCTCGATTTGGG (SEQ ID NO: 166), TTRTTYGGTGGTTGYTTTRTTAA (SEQ ID NO: 169), CCGGACGGTCTTGGTAATTTGGGT (SEQ ID NO: 172), CCGTACGGTTTAGGCAATTTGGGT (SEQ ID NO: 173), AGCTCTTCTATCCTGGTGCTGCG (SEQ ID NO: 176), TGAGACACGGTCCAGACTCCTACG (SEQ ID NO: 179), AAACCGGGCGATATGCGTCTGTAT (SEQ ID NO: 182), TCGTCGCGGAACCATTCGCTAAA (SEQ ID NO: 185), CATCAGGACAAGATGGGCGGTATG (SEQ ID NO: 188), and TGAGACACGGTCCAGACTCCTACG (SEQ ID NO: 191), wherein each probe comprises a fluorophore and/or a fluorescent quencher.

2. (canceled)

3. (canceled)

4. The kit of claim 1, including an endogenous internal control.

5. The kit of claim 4, wherein the endogenous internal control targets a conserved region in Gram-negative bacteria.

6. The kit of claim 1, further comprising the following primers: TGGCCAGAACTGACAGGCAAA (SEQ ID NO: 152), TTTCTCCTGAACGTGGCTGGC (SEQ ID NO: 153), CCGTCACGCTGTTGTTAGG (SEQ ID NO: 155), GCTGTGTTAATCAATGCCACAC (SEQ ID NO: 156), CGTTTCGTCTGGATCGCAC (SEQ ID NO: 158), GCTGGGTAAAATAGGTCACC (SEQ ID NO: 159), GAGAGGATGAYCAGCCACAC (SEQ ID NO: 161), and CGCCCATTGTSCAATATTCC (SEQ ID NO: 162).

7. The kit of claim 6, further comprising the following primers: AATCACAGGGCGTAGTTGTG (SEQ ID NO: 164), ACCCACCAGCCAATCTTAGG (SEQ ID NO: 165), GCGGAGTTAACTATTGGCTAG (SEQ ID NO: 167), GGCCAAGCTTCTATATTTGCG (SEQ ID NO: 168), GCGGAGTTARYTATTGGCTAG (SEQ ID NO: 170), GGCCAAGCYTCTAWATTTGCG (SEQ ID NO: 171), GGCGGCGTTGATGTCCTTCG (SEQ ID NO: 174), CCATTCAGCCAGATCGGCATC (SEQ ID NO: 175), GAGAGGATGAYCAGCCACAC (SEQ ID NO: 177), and CGCCCATTGTSCAATATTCC (SEQ ID NO: 178).

8. The kit of claim 7, further comprising the following primers: AACTTTCACAGGTGTGCTGGGT (SEQ ID NO: 180), CCGTACGCATACTGGCTTTGC (SEQ ID NO: 181), GTATCGCCGTCTAGTTCTGC (SEQ ID NO: 183), CCTTGAATGAGCTGCACAGTGG (SEQ ID NO: 184), GTTTGATCGTCAGGGATGGC (SEQ ID NO: 186), GGCGAAAGTCAGGCTGTG (SEQ ID NO: 187), GAGAGGATGAYCAGCCACAC (SEQ ID NO: 189), and CGCCCATTGTSCAATATTCC (SEQ ID NO: 190).

9. (canceled)

10. (canceled)

11. (canceled)

12. The kit of claim 1, including at least one control DNA mix.

13. The kit of claim 1, including exactly two control DNA mixes.

14. The kit of claim 1, including exactly three control DNA mixes.

15. The kit of claim 1, including a composition containing a tracking dye.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)
